(19) United States
(12) Patent Application Publication
(10) Pub. No.: US 2008/0004270 A1
(43) Pub. Date: Jan. 3, 2008

(54) 3,4-Disubstituted Pyrazoles as Cyclin Dependent Kinases (Cdk) or Aurora Kinase or Glycogen Synthase 3 (Gsk-3) Inhibitors

(75) Inventors: Adrian Liam Gill, Cambridge (GB); Maria Grazia Carr, Cambridge (GB); Michael Alistair O'Brien, Cambridge (GB); Paul Graham Wyatt, Perth (GB); Valerio Berdini, Cambridge (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS LIMITED, Cambridge, UK (UK)

(21) Appl. No.: 11/571,713

(22) PCT Filed: Jul. 5, 2005
(86) PCT No.: PCT/GB05/02629

§ 371 Date: Apr. 13, 2007

(30) Foreign Application Priority Data
Jul. 5, 2004 (GB) 0415082.7
Jul. 5, 2004 (GB) 0415084.3

(51) Int. Cl. A61K 31/5377; A61K 31/4188; A61P 25/28; A61P 35/00; C07D 235/02; C07D 471/02; C07D 265/34; A61P 37/00; A61P 31/12; A61K 31/47

(52) U.S. Cl. 514/234.2; 514/303; 514/393; 544/118; 546/118; 548/303.1

(57) ABSTRACT

The invention provides compounds of the formula (I); or salts or solvates or N-oxides thereof; wherein: R^{q} is selected from groups (a), (b) and (c); the asterisk denoting the point of attachment to the pyrazole ring; X is N or CR⁵; X″ is NR⁴; O, S or S(O); A is a bond or —(CH₂)ₘ—(B)ₙ—; B is C═O, NR^{g}(C═O) or O(C═O) wherein R^{g} is hydrogen or C₁₋₄ hydrocarbyl optionally substituted by hydroxy or C₁₋₄ alkoxy; m is 0, 1 or 2; n is 0 or 1; R⁰ is hydrogen or, together with NR^{g} when present, forms a group —(CH₂)ₚ— wherein p is 2 to 4; and R¹ to R^{6b} are as defined in the description. Compounds of the formula (I) have activity as inhibitors of CDK, aurora and GSK-3 kinases are useful in treating or preventing diseases such as cancers that are mediated by the said kinases.

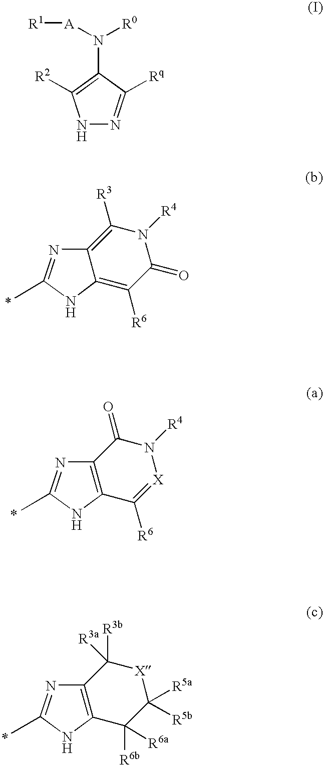

This invention relates to pyrazole compounds that inhibit or modulate the activity of cyclin dependent kinases (CDK) or aurora kinase or glycogen synthase kinase 3 (GSK-3), to the use of the compounds in the treatment or prophylaxis of disease states or conditions mediated by cyclin dependent kinases or aurora kinase or glycogen synthase kinase 3, and to novel compounds having cyclin dependent kinase or aurora kinase or glycogen synthase kinase 3 inhibitory or modulating activity. Also provided are pharmaceutical compositions containing the compounds and novel chemical intermediates.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, disease and conditions of the immune system, disease and conditions of the central nervous system, and angiogenesis.

Cyclin Dependent Kinases

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (CDKs) and a diverse set of their cognate protein partners termed cyclins. CDKs are cdc2 (also known as CDK1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific CDK partner proteins.

Modulation of the expression levels, degradation rates, and activation levels of various CDKs and cyclins throughout the cell cycle leads to the cyclical formation of a series of CDK/cyclin complexes, in which the CDKs are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required CDK/cyclin complex, can lead to cell cycle arrest and/or cellular apoptosis. Aberrant cellular proliferation, as manifested in cancer, can often be attributed to loss of correct cell cycle control. Inhibition of CDK enzymatic activity therefore provides a means by which abnormally dividing cells can have their division arrested and/or be killed. The diversity of CDKs, and CDK complexes, and their critical roles in mediating the cell cycle, provides a broad spectrum of potential therapeutic targets selected on the basis of a defined biochemical rationale.

Progression from the G1 phase to the S phase of the cell cycle is primarily regulated by CDK2, CDK3, CDK4 and CDK6 via association with members of the D and E type cyclins. The D-type cyclins appear instrumental in enabling passage beyond the G1 restriction point, where as the CDK2/cyclin E complex is key to the transition from the G1 to S phase. Subsequent progression through S phase and entry into G2 is thought to require the CDK2/cyclin A complex. Both mitosis, and the G2 to M phase transition which triggers it, are regulated by complexes of CDK1 and the A and B type cyclins.

During G1 phase Retinoblastoma protein (Rb), and related pocket proteins such as p130, are substrates for CDK(2, 4, & 6)/cyclin complexes. Progression through G1 is in part facilitated by hyperphosphorylation, and thus inactivation, of Rb and p130 by the CDK(4/6)/cyclin-D complexes. Hyperphosphorylation of Rb and p130 causes the release of transcription factors, such as E2F, and thus the expression of genes necessary for progression through G1 and for entry into S-phase, such as the gene for cyclin E. Expression of cyclin E facilitates formation of the CDK2/cyclin E complex which amplifies, or maintains, E2F levels via further phosphorylation of Rb. The CDK2/cyclin E complex also phosphorylates other proteins necessary for DNA replication, such as NPAT, which has been implicated in histone biosynthesis. G1 progression and the G1/S transition are also regulated via the mitogen stimulated Myc pathway, which feeds into the CDK2/cyclin E pathway. CDK2 is also connected to the p53 mediated DNA damage response pathway via p53 regulation of p21 levels. p21 is a protein inhibitor of CDK2/cyclin E and is thus capable of blocking, or delaying, the G1/S transition. The CDK2/cyclin E complex may thus represent a point at which biochemical stimuli from the Rb, Myc and p53 pathways are to some degree integrated. CDK2 and/or the CDK2/cyclin E complex therefore represent good targets for therapeutics designed at arresting, or recovering control of, the cell cycle in aberrantly dividing cells.

The exact role of CDK3 in the cell cycle is not clear. As yet no cognate cyclin partner has been identified, but a dominant negative form of CDK3 delayed cells in G1, thereby suggesting that CDK3 has a role in regulating the G1/S transition.

Although most CDKs have been implicated in regulation of the cell cycle there is evidence that certain members of the CDK family are involved in other biochemical processes. This is exemplified by CDK5 which is necessary for correct neuronal development and which has also been implicated in the phosphorylation of several neuronal proteins such as Tau, NUDE-1, synapsin1, DARPP32 and the Munc18/Syntaxin1A complex. Neuronal CDK5 is conventionally activated by binding to the p35/p39 proteins. CDK5 activity can, however, be deregulated by the binding of p25, a truncated version of p35. Conversion of p35 to p25, and subsequent deregulation of CDK5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

CDK7 is a nuclear protein that has cdc2 CAK activity and binds to cyclin H. CDK7 has been identified as component of the TFIIH transcriptional complex which has RNA polymerase II C-terminal domain (CTD) activity. This has been associated with the regulation of HIV-1 transcription via a Tat-mediated biochemical pathway. CDK8 binds cyclin C and has been implicated in the phosphorylation of the CTD of RNA polymerase II. Similarly the CDK9/cyclin-T1 complex (P-TEFb complex) has been implicated in elongation control of RNA polymerase II. PTEF-b is also required for activation of transcription of the HIV-1 genome by the viral transactivator Tat through its interaction with cyclin T1. CDK7, CDK8, CDK9 and the P-TEFb complex are therefore potential targets for anti-viral therapeutics.

At a molecular level mediation of CDK/cyclin complex activity requires a series of stimulatory and inhibitory phosphorylation, or dephosphorylation, events. CDK phosphorylation is performed by a group of CDK activating kinases (CAKs) and/or kinases such as wee1, Myt1 and Mik1. Dephosphorylation is performed by phosphatases such as cdc25(a & c), pp2a, or KAP.

CDK/cyclin complex activity may be further regulated by two families of endogenous cellular proteinaceous inhibitors: the Kip/Cip family, or the INK family. The INK proteins specifically bind CDK4 and CDK6. p16^{ink4} (also known as MTS1) is a potential tumour suppressor gene that is mutated, or deleted, in a large number of primary cancers. The Kip/Cip family contains proteins such as p21^{Cip1,Waf1}, p27^{Kip1} and p57^{kip2}. As discussed previously p21 is induced by p53 and is able to inactivate the CDK2/cyclin(E/A) and CDK4/cyclin(D1/D2/D3) complexes. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of CDKs, and their associated proteins, in co-ordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable. CDK inhibitors could conceivably also be used to treat other conditions such as viral infections, autoimmune diseases and neuro-degenerative diseases, amongst others. CDK targeted therapeutics may also provide clinical benefits in the treatment of the previously described diseases when used in combination therapy with either existing, or new, therapeutic agents. CDK targeted anticancer therapies could potentially have advantages over many current antitumour agents as they would not directly interact with DNA and should therefore reduce the risk of secondary tumour development.

Aurora Kinases

Relatively recently, a new family of serine/threonine kinases known as the aurora kinases has been discovered that are involved in the G2 and M phases of the cell cycle, and which are important regulators of mitosis.

The precise role of aurora kinases has yet to be elucidated but that they play a part in mitotic checkpoint control, chromosome dynamics and cytokinesis (Adams et al., Trends Cell Biol., 11: 49-54 (2001). Aurora kinases are located at the centrosomes of interphase cells, at the poles of the bipolar spindle and in the mid-body of the mitotic apparatus.

Three members of the aurora kinase family have been found in mammals so far (E. A. Nigg, Nat. Rev. Mol. Cell Biol. 2: 21-32, (2001)). These are:

Aurora A (also referred to in the literature as Aurora 2);

Aurora B (also referred to in the literature as Aurora 1); and

Aurora C (also referred to in the literature as Aurora 3).

The aurora kinases have highly homologous catalytic domains but differ considerably in their N-terminal portions (Katayama H, Brinkley W R, Sen S.; The Aurora kinases: role in cell transformation and tumorigenesis; Cancer Metastasis Rev. 2003 December; 22(4):451-64).

The substrates of the aurora kinases A and B have been identified as including a kinesin-like motor protein, spindle apparatus proteins, histone H3 protein, kinetochore protein and the tumour suppressor protein p53.

Aurora A kinases are believed to be involved in spindle formation and become localised on the centrosome during the early G2 phase where they phosphorylate spindle-associated proteins (Prigent et al., Cell, 114: 531-535 (2003). Hirota et al, Cell, 114:585-598, (2003) found that cells depleted of aurora A protein kinase were unable to enter mitosis. Furthermore, it has been found (Adams, 2001) that mutation or disruption of the aurora A gene in various species leads to mitotic abnormalities, including centrosome separation and maturation defects, spindle aberrations and chromosome segregation defects.

The aurora kinases are generally expressed at a low level in the majority of normal tissues, the exceptions being tissues with a high proportion of dividing cells such as the thymus and testis. However, elevated levels of aurora kinases have been found in many human cancers (Giet et al., J. Cell. Sci. 112: 3591-361, (1999) and Katayama (2003). Furthermore, aurora A kinase maps to the chromosome 20q13 region that has frequently been found to be amplified in many human cancers.

Thus, for example, significant aurora A over-expression has been detected in human breast, ovarian and pancreatic cancers (see Zhou et al., Nat. Genet. 20: 189-193, (1998), Tanaka et al., Cancer Res., 59: 2041-2044, (1999) and Han et al., cancer Res., 62: 2890-2896, (2002).

Moreover, Isola, American Journal of Pathology 147, 905-911 (1995) has reported that amplification of the aurora A locus (20q13) correlates with poor prognosis for patients with node-negative breast cancer.

Amplification and/or over-expression of aurora A is observed in human bladder cancers and amplification of aurora A is associated with aneuploidy and aggressive clinical behaviour, see Sen et al., J. Natl. Cancer Inst, 94: 1320-1329 (2002).

Elevated expression of aurora A has been detected in over 50% of colorectal cancers, (see Bischoff et al., EMBO J., 17: 3052-3065, (1998) and Takahashi et al., Jpn. J. Cancer Res., 91: 1007-1014 (2000)) ovarian cancers (see Gritsko et al. Clin. Cancer Res., 9: 1420-1426 (2003), and gastric tumours Sakakura et al., British Journal of Cancer, 84: 824-831 (2001).

Tanaka et al. Cancer Research, 59: 2041-2044 (1999) found evidence of over-expression of aurora A in 94% of invasive duct adenocarcinomas of the breast. High levels of aurora A kinase have also been found in renal, cervical, neuroblastoma, melanoma, lymphoma, pancreatic and prostate tumour cell lines Bischoff et al. (1998), EMBO J., 17: 3052-3065 (1998); Kimura et al. J. Biol. Chem., 274: 7334-7340 (1999); Zhou et al., Nature Genetics, 20: 189-193 (1998); Li et al., Clin Cancer Res. 9 (3): 991-7 (2003)].

Aurora B is highly expressed in multiple human tumour cell lines, including leukemic cells [Katayama et al., Gene 244: 1-7)]. Levels of this enzyme increase as a function of Duke's stage in primary colorectal cancers [Katayama et al., J. Natl Cancer Inst., 91: 1160-1162 (1999)].

High levels of aurora 3 (aurora C) have been detected in several tumour cell lines, even though this kinase tends to be restricted to germ cells in normal tissues (see Kimura et al. Journal of Biological Chemistry, 274: 7334-7340 (1999)). Over-expression of Aurora-3 in approximately 50% of colorectal cancers has also been reported in the article by Takahashi et al., Jpn J. Cancer Res. 91: 1007-1014 (2001)].

Other reports of the role of aurora kinases in proliferative disorders may be found in Bischoff et al., Trends in Cell Biology 9: 454-459 (1999); Giet et al. Journal of Cell Science, 112: 3591-3601 (1999) and Dutertre, et al. Oncogene, 21: 6175-6183 (2002).

Royce et al report that the expression of the aurora 2 gene (known as STK15 or BTAK) has been noted in approximately one-fourth of primary breast tumours. (Royce M E, Xia W, Sahin A A, Katayama H, Johnston D A, Hortobagyi G, Sen S, Hung M C; STK15/Aurora-A expression in primary breast tumours is correlated with nuclear grade but not with prognosis; Cancer. 2004 Jan. 1; 100(1):12-9).

Endometrial carcinoma (EC) comprises at least two types of cancer: endometrioid carcinomas (EECs) are estrogen-related tumours, which are frequently euploid and have a good prognosis. Nonendometrioid carcinomas (NEECs; serous and clear cell forms) are not estrogen related, are frequently aneuploid, and are clinically aggressive. It has also been found that aurora was amplified in 55.5% of NEECs but not in any EECs (P<or=0.001) (Moreno-Bueno G, Sanchez-Estevez C, Cassia R, Rodriguez-Perales S, Diaz-Uriarte R, Dominguez O, Hardisson D, Andujar M, Prat J, Matias-Guiu X, Cigudosa J C, Palacios J. Cancer Res. 2003 Sep. 15; 63(18):5697-702).

Reichardt et al (Oncol Rep. 2003 September-October; 10(5): 1275-9) have reported that quantitative DNA analysis by PCR to search for aurora amplification in gliomas revealed that five out of 16 tumours (31%) of different WHO grade (1× grade II, 1× grade III, 3× grade IV) showed DNA amplification of the aurora 2 gene. It was hypothesized that amplification of the aurora 2 gene may be a non-random genetic alteration in human gliomas playing a role in the genetic pathways of tumourigenesis.

Results by Hamada et al (Br. J. Haematol. 2003 May; 121(3):439-47) also suggest that aurora 2 is an effective candidate to indicate not only disease activity but also tumourigenesis of non-Hodgkin's lymphoma. Retardation of tumour cell growth resulting from the restriction of this gene's functions could be a therapeutic approach for non-Hodgkin's lymphoma.

In a study by Gritsko et al (Clin Cancer Res. 2003 April; 9(4):1420-6)), the kinase activity and protein levels of aurora A were examined in 92 patients with primary ovarian tumours. In vitro kinase analyses revealed elevated aurora A kinase activity in 44 cases (48%). Increased aurora A protein levels were detected in 52 (57%) specimens. High protein levels of aurora A correlated well with elevated kinase activity.

Results obtained by Li et al (Clin. Cancer Res. 2003 March; 9(3):991-7) showed that the aurora A gene is overexpressed in pancreatic tumours and carcinoma cell lines and suggest that overexpression of aurora A may play a role in pancreatic carcinogenesis.

Similarly, it has been shown that aurora A gene amplification and associated increased expression of the mitotic kinase it encodes are associated with aneuploidy and aggressive clinical behaviour in human bladder cancer. (J. Natl. Cancer Inst. 2002 Sep. 4; 94(17):1320-9).

Investigation by several groups (Dutertre S, Prigent C., Aurora-A overexpression leads to override of the microtubule-kinetochore attachment checkpoint; Mol. Interv. 2003 May; 3(3):127-30 and Anand S, Penrhyn-Lowe S, Venkitaraman A R., aurora A amplification overrides the mitotic spindle assembly checkpoint, inducing resistance to Taxol, Cancer Cell. 2003 January; 3(1):51-62) suggests that overexpression of aurora kinase activity is associated with resistance to some current cancer therapies. For example overexpression of aurora A in mouse embryo fibroblasts can reduce the sensitivity of these cells to the cytotoxic effects of taxane derivatives. Therefore aurora kinase inhibitors may find particular use in patients who have developed resistance to existing therapies.

On the basis of work carried out to date, it is envisaged that inhibition of Aurora kinases, particularly aurora kinase A or aurora kinase B, will prove an effective means of arresting tumour development.

Harrington et al (Nat. Med. 2004 March; 10(3):262-7) have demonstrated that an inhibitor of the aurora kinases suppresses tumour growth and induces tumour regression in vivo. In the study, the aurora kinase inhibitor blocked cancer cell proliferation, and also triggered cell death in a range of cancer cell lines including leukaemic, colorectal and breast cell lines.

Cancers which may be particularly amenable to aurora inhibitors include breast, bladder, colorectal, pancreatic and ovarian cancers, non-Hodgkin's lymphoma, gliomas and nonendometrioid endometrial carcinomas.

Glycogen Synthase Kinase-3

Glycogen Synthase Kinase-3 (GSK3) is a serine-threonine kinase that occurs as two ubiquitously expressed isoforms in humans (GSK3α & beta GSK3β). GSK3 has been implicated as having roles in embryonic development, protein synthesis, cell proliferation, cell differentiation, microtubule dynamics, cell motility and cellular apoptosis. As such GSK3 has been implicated in the progression of disease states such as diabetes, cancer, Alzheimer's disease, stroke, epilepsy, motor neuron disease and/or head trauma. Phylogenetically GSK3 is most closely related to the cyclin dependent kinases (CDKs).

The consensus peptide substrate sequence recognised by GSK3 is (Ser/Thr)-X-X-X-(pSer/pThr), where X is any amino acid (at positions (n+1), (n+2), (n+3)) and pSer and pThr are phospho-serine and phospho-threonine respectively (n+4). GSK3 phosphorylates the first serine, or threonine, at position (n). Phospho-serine, or phospho-threonine, at the (n+4) position appear necessary for priming GSK3 to give maximal substrate turnover. Phosphorylation of GSK3α at Ser21, or GSK3β at Ser9, leads to inhibition of GSK3. Mutagenesis and peptide competition studies have led to the model that the phosphorylated N-terminus of GSK3 is able to compete with phospho-peptide substrate (S/TXXXpS/pT) via an autoinhibitory mechanism. There are also data suggesting that GSK3α and GSKβ may be subtly regulated by phosphorylation of tyrosines 279 and 216 respectively. Mutation of these residues to a Phe caused a reduction in in vivo kinase activity. The X-ray crystallographic structure of GSK3β has helped to shed light on all aspects of GSK3 activation and regulation.

GSK3 forms part of the mammalian insulin response pathway and is able to phosphorylate, and thereby inactivate, glycogen synthase. Upregulation of glycogen synthase activity, and thereby glycogen synthesis, through inhibition of GSK3, has thus been considered a potential means of combating type II, or non-insulin-dependent diabetes mellitus (NIDDM): a condition in which body tissues become resistant to insulin stimulation. The cellular insulin response in liver, adipose, or muscle tissues, is triggered by insulin binding to an extracellular insulin receptor. This causes the phosphorylation, and subsequent recruitment to the plasma membrane, of the insulin receptor substrate (IRS) proteins. Further phosphorylation of the IRS proteins initiates recruitment of phosphoinositide-3 kinase (PI3K) to the plasma membrane where it is able to liberate the second messenger phosphatidylinosityl 3,4,5-trisphosphate (PIP3). This facilitates co-localisation of 3-phosphoinositide-dedependent protein kinase 1 (PDK1) and protein kinase B (PKB or Akt) to the membrane, where PDK1 activates PKB. PKB is able to phosphorylate, and thereby inhibit, GSK3α and/or GSKβ through phosphorylation of Ser9, or ser21, respectively. The inhibition of GSK3 then triggers upregulation of glycogen synthase activity. Therapeutic agents able to inhibit GSK3 may thus be able to induce cellular responses akin to those seen on insulin stimulation. A further in vivo substrate of GSK3 is the eukaryotic protein synthesis initiation factor 2B (eIF2B). eIF2B is inactivated via phosphorylation and is thus able to suppress protein biosynthesis. Inhibition of GSK3, e.g. by inactivation of the “mammalian target of rapamycin” protein (mTOR), can thus upregulate protein biosynthesis. Finally there is some evidence for regulation of GSK3 activity via the mitogen activated protein kinase (MAPK) pathway through phosphorylation of GSK3 by kinases such as mitogen activated protein kinase activated protein kinase 1 (MAPKAP-K1 or RSK). These data suggest that GSK3 activity may be modulated by mitogenic, insulin and/or amino acid stimuli.

It has also been shown that GSK3β is a key component in the vertebrate Wnt signalling pathway. This biochemical pathway has been shown to be critical for normal embryonic development and regulates cell proliferation in normal tissues. GSK3 becomes inhibited in response to Wnt stimuli. This can lead to the de-phosphorylation of GSK3 substrates such as Axin, the adenomatous polyposis coli (APC) gene product and β-catenin. Aberrant regulation of the Wnt pathway has been associated with many cancers. Mutations in APC, and/or β-catenin, are common in colorectal cancer and other tumours. β-catenin has also been shown to be of importance in cell adhesion. Thus GSK3 may also modulate cellular adhesion processes to some degree. Apart from the biochemical pathways already described there are also data implicating GSK3 in the regulation of cell division via phosphorylation of cyclin-D1, in the phosphorylation of transcription factors such as c-Jun, CCAAT/enhancer binding protein α (C/EBPα), c-Myc and/or other substrates such as Nuclear Factor of Activated T-cells (NFATc), Heat Shock Factor-1 (HSF-1) and the c-AMP response element binding protein (CREB). GSK3 also appears to play a role, albeit tissue specific, in regulating cellular apoptosis. The role of GSK3 in modulating cellular apoptosis, via a pro-apoptotic mechanism, may be of particular relevance to medical conditions in which neuronal apoptosis can occur. Examples of these are head trauma, stroke, epilepsy, Alzheimer's and motor neuron diseases, progressive supranuclear palsy, corticobasal degeneration, and Pick's disease. In vitro it has been shown that GSK3 is able to hyper-phosphorylate the microtubule associated protein Tau. Hyperphosphorylation of Tau disrupts its normal binding to microtubules and may also lead to the formation of intra-cellular Tau filaments. It is believed that the progressive accumulation of these filaments leads to eventual neuronal dysfunction and degeneration. Inhibition of Tau phosphorylation, through inhibition of GSK3, may thus provide a means of limiting and/or preventing neurodegenerative effects.

Diffuse Large B-Cell Lymphomas (DLBCL)

Cell cycle progression is regulated by the combined action of cyclins, cyclin-dependent kinases (CDKs), and CDK-inhibitors (CDKi), which are negative cell cycle regulators. p27KIP1 is a CDKi key in cell cycle regulation, whose degradation is required for G1/S transition. In spite of the absence of p27KIP1 expression in proliferating lymphocytes, some aggressive B-cell lymphomas have been reported to show an anomalous p27KIP1 staining. An abnormally high expression of p27KIP1 was found in lymphomas of this type. Analysis of the clinical relevance of these findings showed that a high level of p27KIP1 expression in this type of tumour is an adverse prognostic marker, in both univariate and multivariate analysis. These results show that there is abnormal p27KIP1 expression in Diffuse Large B-cell Lymphomas (DLBCL), with adverse clinical significance, suggesting that this anomalous p27KIP1 protein may be rendered non-functional through interaction with other cell cycle regulator proteins. (Br. J. Cancer. 1999 July; 80(9):1427-34. p27KIP1 is abnormally expressed in Diffuse Large B-cell Lymphomas and is associated with an adverse clinical outcome. Saez A, Sanchez E, Sanchez-Beato M, Cruz M A, Chacon I, Munoz E, Camacho F I, Martinez-Montero J C, Mollejo M, Garcia J F, Piris M A. Department of Pathology, Virgen de la Salud Hospital, Toledo, Spain.)

Chronic Lymphocytic Leukemia

B-Cell chronic lymphocytic leukaemia (CLL) is the most common leukaemia in the Western hemisphere, with approximately 10,000 new cases diagnosed each year (Parker S L, Tong T, Bolden S, Wingo P A: Cancer statistics, 1997. Ca. Cancer. J. Clin. 47:5, (1997)). Relative to other forms of leukaemia, the overall prognosis of CLL is good, with even the most advanced stage patients having a median survival of 3 years.

The addition of fludarabine as initial therapy for symptomatic CLL patients has led to a higher rate of complete responses (27% ν3%) and duration of progression-free survival (33 ν17 months) as compared with previously used alkylator-based therapies. Although attaining a complete clinical response after therapy is the initial step toward improving survival in CLL, the majority of patients either do not attain complete remission or fail to respond to fludarabine. Furthermore, all patients with CLL treated with fludarabine eventually relapse, making its role as a single agent purely palliative (Rai K R, Peterson B, Elias L, Shepherd L, Hines J, Nelson D, Cheson B, Kolitz J, Schiffer C A: A randomized comparison of fludarabine and chlorambucil for patients with previously untreated chronic lymphocytic leukemia. A CALGB SWOG, CTG/NCI-C and ECOG Inter-Group Study. Blood 88:141a, 1996 (abstr 552, suppl 1). Therefore, identifying new agents with novel mechanisms of action that complement fludarabine's cytotoxicity and abrogate the resistance induced by intrinsic CLL drug-resistance factors will be necessary if further advances in the therapy of this disease are to be realized.

The most extensively studied, uniformly predictive factor for poor response to therapy and inferior survival in CLL patients is aberrant p53 function, as characterized by point mutations or chromosome 17p 13 deletions. Indeed, virtually no responses to either alkylator or purine analog therapy have been documented in multiple single institution case series for those CLL patients with abnormal p53 function. Introduction of a therapeutic agent that has the ability to overcome the drug resistance associated with p53 mutation in CLL would potentially be a major advance for the treatment of the disease.

Flavopiridol and CYC 202, inhibitors of cyclin-dependent kinases induce in vitro apoptosis of malignant cells from B-cell chronic lymphocytic leukemia (B-CLL).

Flavopiridol exposure results in the stimulation of caspase 3 activity and in caspase-dependent cleavage of p27(kip1), a negative regulator of the cell cycle, which is overexpressed in B-CLL (Blood. 1998 Nov. 15; 92(10):3804-16 Flavopiridol induces apoptosis in chronic lymphocytic leukemia cells via activation of caspase-3 without evidence of bcl-2 modulation or dependence on functional p53. Byrd J C, Shinn C, Waselenko J K, Fuchs E J, Lehman T A, Nguyen P L, Flinn I W, Diehl L F, Sausville E, Grever M R).

### PRIOR ART

WO 02/34721 from Du Pont discloses a class of indeno [1,2-c]pyrazol-4-ones as inhibitors of cyclin dependent kinases.

WO 01/81348 from Bristol Myers Squibb describes the use of 5-thio-, sulfinyl- and sulfonylpyrazolo[3,4-b]-pyridines as cyclin dependent kinase inhibitors.

WO 00/62778 also from Bristol Myers Squibb discloses a class of protein tyrosine kinase inhibitors.

WO 01/72745A1 from Cyclacel describes 2-substituted 4-heteroaryl-pyrimidines and their preparation, pharmaceutical compositions containing them and their use as inhibitors of cyclin-dependant kinases (CDKs) and hence their use in the treatment of proliferative disorders such as cancer, leukaemia, psoriasis and the like.

WO 99/21845 from Agouron describes 4-aminothiazole derivatives for inhibiting cyclin-dependent kinases (CDKs), such as CDK1, CDK2, CDK4, and CDK6. The invention is also directed to the therapeutic or prophylactic use of pharmaceutical compositions containing such compounds and to methods of treating malignancies and other disorders by administering effective amounts of such compounds.

WO 01/53274 from Agouron discloses as CDK kinase inhibitors a class of compounds which can comprise an amide-substituted benzene ring linked to an N-containing heterocyclic group. Although indazole compounds are not mentioned generically, one of the exemplified compounds comprises an indazole 3-carboxylic acid anilide moiety linked via a methylsulfanyl group to a pyrazolopyrimidine.

WO 01/98290 (Pharmacia & Upjohn) discloses a class of 3-aminocarbonyl-2-carboxamido thiophene derivatives as protein kinase inhibitors. The compounds are stated to have multiple protein kinase activity.

WO 01/53268 and WO 01/02369 from Agouron disclose compounds that mediate or inhibit cell proliferation through the inhibition of protein kinases such as cyclin dependent kinase or tyrosine kinase. The Agouron compounds have an aryl or heteroaryl ring attached directly or though a CH═CH or CH═N group to the 3-position of an indazole ring.

WO 00/59902, WO 00/39108 and WO 02/00651 (each to Du Pont Pharmaceuticals) describe broad classes of heterocyclic compounds that are inhibitors of trypsin-like serine protease enzymes, especially factor Xa and thrombin. The compounds are stated to be useful as anticoagulants or for the prevention of thromboembolic disorders.

Heterocyclic compounds that have activity against factor Xa are also disclosed in WO 02/26712 and WO 01/1978 Cor Therapeutics) and US 2002/0091116 (Zhu et al.).

WO 00/39127 (Dupont) discloses 1H-imidazo[4,5-d]pyridazin-7-ones, 3H-imidazo[4,5-c]pyrid-4-ones and their corresponding thiones as corticotrophin releasing factor receptor ligands.

WO 98/47885, WO 98/50343 and WO 00/06575 (SmithKline Beecham) relate to bicycle heterocyclic compounds for the treatment of CNS disorders.

EP 1193255 relates to heterocyclic compounds for treating inflammatory diseases.

WO 98/00401, WO 98/00144 and WO 98/00134 (Merck) relate to compounds as fibrinogen receptor antagonists and prodrugs.

WO 03/066629 discloses heteroaryl compounds which are inhibitors of GSK-3 and Lck kinases.

WO 03/035065 (Aventis) discloses a broad class of benzimidazole derivatives as protein kinase inhibitors but does not disclose activity against CDK kinases or aurora kinases or GSK kinases.

WO 97/36585 and U.S. Pat. No. 5,874,452 (both to Merck) disclose biheteroaryl compounds that are inhibitors of farnesyl transferase.

Wo 2005/047266 (Lorus) discloses aryl imidazoles and fused imidazoles as anti-cancer agents.

### SUMMARY OF THE INVENTION

The invention provides compounds that have cyclin dependent kinase inhibiting or modulating activity and/or glycogen synthase kinase-3 (GSK3) inhibiting or modulating activity, and/or aurora kinase inhibiting or modulating activity, and which it is envisaged will be useful in preventing or treating disease states or conditions mediated by the kinases.

Accordingly, in one aspect, the invention provides a compound of the formula (I): 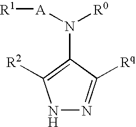
or salts or solvates or N-oxides thereof;
wherein
R^{q} is selected from groups (a), (b) and (c): 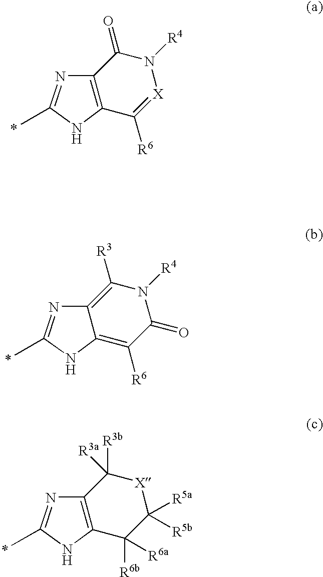
the asterisk denoting the point of attachment to the pyrazole ring;
X is N or CR⁵;
X″ is NR^{4′}, O, S or S(O);
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³, R⁵ and R⁶ are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; and a group R^{a}-R^{b};
R^{3a}, R^{3b}, R^{5a}, and R^{5b} are the same or different and each is selected from hydrogen, trifluoromethyl, cyano, carboxy, carbocyclic and heterocyclic groups having from 3 to 12 ring members; and a group R^{a}-R^{b};
R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹
R^{4′} is selected from hydrogen, trifluoromethyl, carbocyclic and heterocyclic groups having from 3 to 12 ring members; and a group R^{d′}-R^{e′} wherein R^{d′} is a bond, CO, C(X²)X¹, SO, SO₂, or SO₂NR^{c}; and R^{e′} is selected from carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{6a} and R^{6b} are the same or different and each is selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; and a group R^{a}-R^{b};
R^{a} is a bond, O, CO, X¹C(X²), C(X²)X¹, X¹C(X²)X¹, S, SO, SO₂, NR^{c}, SO₂NR^{c} or NR^{c}SO₂;

R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═0, ═S or ═NR^{c}.

In one embodiment, the invention provides compounds of the formulae (Ia) and (Ib): 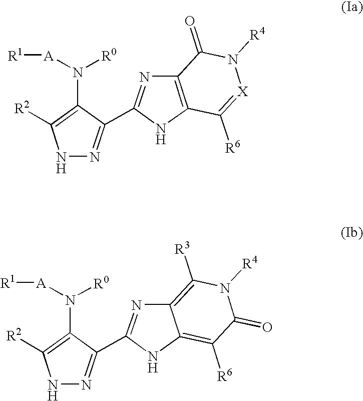
or salts or solvates or N-oxides thereof;
wherein
X is N or CR⁵;
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³, R⁵ and R⁶ are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

In another embodiment, the invention provides a compound of the formula (Ic): 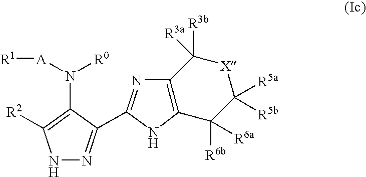
or salts or solvates or N-oxides thereof;
wherein
X″ is NR^{4′}, O, S or S(O);
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R^{3a}, R^{3b}, R^{5a}, and R^{5b} are the same or different and each is selected from hydrogen, trifluoromethyl, cyano, carboxy, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
R^{4′} is selected from hydrogen, trifluoromethyl, carbocyclic and heterocyclic groups having from 3 to 12 ring members; a group R^{d′}-R^{e′} wherein R^{d′} is a bond, CO, C(X²)X¹, SO, SO₂, or SO₂NR^{c}; and R^{e′} is selected from carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{6a} and R^{6b} are the same or different and each is selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

In addition to having cyclin dependent kinase inhibiting or modulating activity, or glycogen synthase kinase-3 (GSK3) inhibiting or modulating activity, or aurora kinase inhibiting or modulating activity, the compounds according to the present invention have advantageous physiochemical and pharmacokinetic properties, including, for example, low plasma clearance, a low propensity to inhibit P450's in vitro, low plasma protein binding and/or a high level of solubility as well as demonstrating a good kinase selectivity profile and/or an even better cellular activity.

In further aspects, the invention provides:
A compound of the formula (I) as defined herein for use in the prophylaxis or treatment of a disease state or condition mediated by a cyclin dependent kinase or glycogen synthase kinase-3 or an aurora kinase.
The use of a compound of the formula (I) as defined herein for the manufacture of a medicament for the prophylaxis or treatment of a disease state or condition mediated by a cyclin dependent kinase or glycogen synthase kinase-3 or an aurora kinase.
A method for the prophylaxis or treatment of a disease state or condition mediated by a cyclin dependent kinase or glycogen synthase kinase-3 or an aurora kinase, which method comprises administering to a subject in need thereof a compound of the formula (I) as defined herein.
A method for alleviating or reducing the incidence of a disease state or condition mediated by a cyclin dependent kinase or glycogen synthase kinase-3 or an aurora kinase, which method comprises administering to a subject in need thereof a compound of the formula (I) as defined herein.
A method for treating a disease or condition comprising or arising from abnormal cell growth in a mammal, which method comprises administering to the mammal a compound of the formula (I) as defined herein in an amount effective in inhibiting abnormal cell growth.
A method for alleviating or reducing the incidence of a disease or condition comprising or arising from abnormal cell growth in a mammal, which method comprises administering to the mammal a compound of the formula (I) as defined herein in an amount effective in inhibiting abnormal cell growth.
A method for treating a disease or condition comprising or arising from abnormal cell growth in a mammal, the method comprising administering to the mammal a compound of the formula (I) as defined herein in an amount effective to inhibit CDK 1 and/or 2 or glycogen synthase kinase-3 or aurora kinase (e.g. aurora A kinase or aurora B kinase) activity.
A method for alleviating or reducing the incidence of a disease or condition comprising or arising from abnormal cell growth in a mammal, the method comprising administering to the mammal a compound of the formula (I) as defined herein in an amount effective to inhibit a cdk kinase (such as cdk1 or cdk2) or glycogen synthase kinase-3 or aurora kinase (such as aurora A kinase or aurora B kinase) activity.
A method of inhibiting a cyclin dependent kinase or glycogen synthase kinase-3 or an aurora kinase, which method comprises contacting the kinase with a kinase-inhibiting compound of the formula (I) as defined herein.
A method of modulating a cellular process (for example cell division) by inhibiting the activity of a cyclin dependent kinase or glycogen synthase kinase-3 or an aurora kinase using a compound of the formula (I) as defined herein.
The use of a compound of the formula (I) as defined herein for the manufacture of a medicament for prophylaxis or treatment of a disease or condition characterised by up-regulation of an aurora kinase (e.g. aurora A kinase or aurora B kinase).
The use of a compound of the formula (I) as defined herein for the manufacture of a medicament for the prophylaxis or treatment of a cancer, the cancer being one which is characterised by up-regulation of an aurora kinase (e.g. aurora A kinase or aurora B kinase).
The use of a compound of the formula (I) as defined herein for the manufacture of a medicament for the prophylaxis or treatment of cancer in a patient selected from a sub-population possessing the Ile31 variant of the aurora A gene.
The use of a compound of the formula (I) as defined herein for the manufacture of a medicament for the prophylaxis or treatment of cancer in a patient who has been diagnosed as forming part of a sub-population possessing the Ile31 variant of the aurora A gene.
A method for the prophylaxis or treatment of a disease or condition characterised by up-regulation of an aurora kinase (e.g. aurora A kinase or aurora B kinase), the method comprising administering a compound of the formula (I) as defined herein.
A method for alleviating or reducing the incidence of a disease or condition characterised by up-regulation of an aurora kinase (e.g. aurora A kinase or aurora B kinase), the method comprising administering a compound of the formula (I) as defined herein.
A method for the prophylaxis or treatment of (or alleviating or reducing the incidence of) cancer in a patient suffering from or suspected of suffering from cancer; which method comprises (i) subjecting a patient to a diagnostic test to determine whether the patient possesses the Ile31 variant of the aurora A gene; and (ii) where the patient does possess the said variant, thereafter administering to the patient a compound of the formula (I) as defined herein having aurora kinase inhibiting activity.
A method for the prophylaxis or treatment of (or alleviating or reducing the incidence of) a disease state or condition characterised by up-regulation of an aurora kinase (e.g. aurora A kinase or aurora B kinase); which method comprises (i) subjecting a patient to a diagnostic test to detect a marker characteristic of up-regulation of the aurora kinase and (ii) where the diagnostic test is indicative of up-regulation of aurora kinase, thereafter administering to the patient a compound of the formula (I) as defined herein having aurora kinase inhibiting activity.
A pharmaceutical composition comprising a novel compound of the formula (I) as hereinbefore defined and a pharmaceutically acceptable carrier.
A compound of the formula (I) for use in medicine.

In the foregoing aspects of the invention, and elsewhere in this application, unless the context requires otherwise, references to formula (I) shall be taken to apply also to formulae (Ia), (Ib), (Ic), (II), (III), (IV) and any other sub-groups, embodiments, preferences or examples thereof, as defined herein.

General Preferences and Definitions

The following general preferences and definitions shall apply to each of the moieties X, X″, X¹, X², A, B, R^{a}, R^{b}, R^{c}, R^{d}, R^{e}, R^{d′}, R^{e′}, R^{q}, R⁰ to R^{6b} and any sub definition, sub-group or embodiment thereof, unless the context indicates otherwise.

References to “carbocyclic” and “heterocyclic” groups as used herein, either in the context of R¹ to R^{6b} and R^{b} and sub-definitions thereof or otherwise shall, unless the context indicates otherwise, include both aromatic and non-aromatic ring systems. Thus, for example, the term “carbocyclic and heterocyclic groups having from 3 to 12 ring members” includes within its scope aromatic, non-aromatic, unsaturated, partially saturated and fully saturated carbocyclic and heterocyclic ring systems.

The carbocyclic or heterocyclic groups can be aryl or heteroaryl groups having from 5 to 12 ring members, more usually from 5 to 10 ring members. The term “aryl” as used herein refers to a carbocyclic group having aromatic character and the term “heteroaryl” is used herein to denote a heterocyclic group having aromatic character. The terms “aryl” and “heteroaryl” embrace polycyclic (e.g. bicyclic) ring systems wherein one or more rings are non-aromatic, provided that at least one ring is aromatic. In such polycyclic systems, the group may be attached by the aromatic ring, or to a non-aromatic ring. The aryl or heteroaryl groups can be monocyclic or bicyclic groups and can be unsubstituted or substituted with one or more substituents, for example one or more groups R¹⁰ as defined herein.

The term non-aromatic group embraces unsaturated ring systems without aromatic character, partially saturated and fully saturated carbocyclic and heterocyclic ring systems. The terms “unsaturated” and “partially saturated” refer to rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include cycloalkyl groups as defined below. Partially saturated carbocyclic groups include cycloalkenyl groups as defined below, for example cyclopentenyl, cycloheptenyl and cyclooctenyl.

Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl groups include but are not limited to pyridine, pyrrole, furan, thiophene, imidazole, oxazole, oxadiazole, oxatriazole, isoxazole, thiazole, isothiazole, pyrazole, pyrazine, pyridazine, pyrimidine, triazine, triazole, tetrazole, quinoline, isoquinoline, benzofuran, benzothiophene, chroman, thiochroman, benzimidazole, benzoxazole, benzoisoxazole, benzthiazole, benzisothiazole, isobenzofuran, indole, isoindole, indolizine, indoline, isoindoline, purine (e.g., adenine, guanine), indazole, benzodioxole, chromene, isochromene, chroman, isochroman, benzodioxan, quinolizine, benzoxazine, benzodiazine, pyridopyridine, pyrazolopyridine, quinoxaline, quinazoline, cinnoline, phthalazine, naphthyridine and pteridine groups.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

Examples of six membered heteroaryl groups include but are not limited to pyridine, pyrazine, pyridazine, pyrimidine and triazine.

A bicyclic heteroaryl group may be, for example, a group selected from:
a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a a 5- or 6-membered ring containing 1, 2 or 3 ring heteroatoms;
e) a pyrazole ring fused to a a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered ring containing 1, 2 or 3 ring heteroatoms.

One sub-group of bicyclic heteroaryl groups consists of groups a) to e) and g) to o) above.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzfuran, benzthiophene, benzimidazole, benzoxazole, isobenzoxazole, benzisoxazole, benzthiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), triazolopyrimidine (e.g. [1,2,4]triazolo[1,5-a]pyrimidine), benzodioxole and pyrazolopyridine (e.g. pyrazolo[1,5-a]pyridine) groups.

Particular examples of bicyclic heteroaryl groups containing two fused six membered rings include but are not limited to quinoline, isoquinoline, chroman, thiochroman, chromene, isochromene, chroman, isochroman, benzodioxan, quinolizine, benzoxazine, benzodiazine, pyridopyridine, quinoxaline, quinazoline, cinnoline, phthalazine, naphthyridine and pteridine groups.

Examples of polycyclic aryl and heteroaryl groups containing an aromatic ring and a non-aromatic ring include tetrahydronaphthalene, tetrahydroisoquinoline, tetrahydroquinoline, dihydrobenzothiene, dihydrobenzofuran, 2,3-dihydro-benzo[1,4]dioxine, benzo[1,3]dioxole, 4,5,6,7-tetrahydrobenzofuran, indoline and indane groups.

Examples of carbocyclic aryl groups include phenyl, naphthyl, indenyl, and tetrahydronaphthyl groups.

Examples of non-aromatic heterocyclic groups are groups having from 3 to 12 ring members, more usually 5 to 10 ring members. Such groups can be monocyclic or bicyclic, for example, and typically have from 1 to 5 heteroatom ring members (more usually 1, 2, 3 or 4 heteroatom ring members), usually selected from nitrogen, oxygen and sulphur. The sulphur heteroatom may be oxidized to an SO or SO₂ group. The heterocylic groups can contain, for example, cyclic ether moieties (e.g as in tetrahydrofuran and dioxane), cyclic thioether moieties (e.g. as in tetrahydrothiophene and dithiane), cyclic amine moieties (e.g. as in pyrrolidine), cyclic amides, cyclic esters, cyclic thioamides and cyclic thioesters (as in rings containing the groups X¹C(X²) or C(X²)X¹ e.g. tetrahydropyranone, dihydropyranone, dihydropyridinone, dihydropyridine-thione, tetrahydropyran-thione), cyclic sulphones (e.g. as in sulfolane and sulfolene), cyclic sulphoxides, cyclic sulphonamides and combinations thereof (e.g. thiomorpholine or thiomorpholine 1,1-dioxide).

Particular examples include morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, pyran (2H-pyran or 4H-pyran), dihydrothiophene, dihydropyran, dihydrofuran, dihydrothiazole, tetrahydrofuran, tetrahydrothiophene, dioxane, tetrahydropyran (e.g. 4-tetrahydro pyranyl), imidazoline, imidazolidinone, oxazoline, thiazoline, 2-pyrazoline, pyrazolidine, piperazine, and N-alkyl piperazines such as N-methyl piperazine. In general, preferred non-aromatic heterocyclic groups include morpholine, and N-alkyl piperazines.

It is preferred that heterocyclic group is a monocyclic non-aromatic group containing at least one nitrogen atom, for example up to three nitrogen atoms, preferably 0, 1 or 2 nitrogen atoms. The groups are optionally substituted by 1, 2 or 3 substituent groups R¹⁰ as defined herein.

Examples of non-aromatic carbocyclic groups include cycloalkane groups such as cyclohexyl and cyclopentyl, cycloalkenyl groups such as cyclopentenyl, cyclohexenyl, cycloheptenyl and cyclooctenyl, as well as cyclohexadienyl, cyclooctatetraene, tetrahydronaphthenyl and decalinyl.

Where reference is made herein to carbocyclic and heterocyclic groups, the carbocyclic or heterocyclic ring can, unless the context indicates otherwise, be unsubstituted or substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; or two adjacent groups R¹⁰, together with the carbon atoms or heteroatoms to which they are attached may form a 5-membered heteroaryl ring or a 5- or 6-membered non-aromatic heterocyclic ring, wherein the said heteroaryl and heterocyclic groups contain up to 3 heteroatom ring members selected from N, O and S;
R^{c} is selected from hydrogen and C₁₋₄ hydrocarbyl; and
X¹ is O, S or NR^{c} and X² is ═O, ═S or ═NR^{c}.

Where the substituent group R¹⁰ comprises or includes a carbocyclic or heterocyclic group, the said carbocyclic or heterocyclic group may be unsubstituted or may itself be substituted with one or more further substituent groups R¹⁰. In one sub-group of compounds of the formula (I), such further substituent groups R¹⁰ may include carbocyclic or heterocyclic groups, which are typically not themselves further substituted. In another sub-group of compounds of the formula (I), the said further substituents do not include carbocyclic or heterocyclic groups but are otherwise selected from the groups listed above in the definition of R¹⁰.

Where the carbocyclic and heterocyclic groups have a pair of substituents on adjacent ring atoms, the two substituents may be linked so as to form a cyclic group. For example, an adjacent pair of substituents on adjacent carbon atoms of a ring may be linked via one or more heteroatoms and optionally substituted alkylene groups to form a fused oxa-, dioxa-, aza-, diaza- or oxa-aza-cycloalkyl group. Examples of such linked substituent groups include

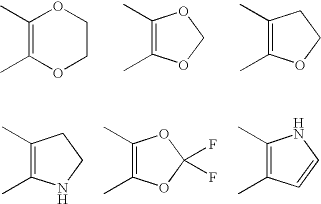

Examples of halogen substituents include fluorine, chlorine, bromine and iodine. Fluorine and chlorine are particularly preferred.

In the definition of the compounds of the formula (I) above and as used hereinafter, the term “hydrocarbyl” is a generic term encompassing aliphatic, alicyclic and aromatic groups having an all-carbon backbone, except where otherwise stated. In certain cases, as defined herein, one or more of the carbon atoms making up the carbon backbone may be replaced by a specified atom or group of atoms. Examples of such groups include alkyl, cycloalkyl, cycloalkenyl, carbocyclic aryl, alkenyl, alkynyl, cycloalkylalkyl, cycloalkenylalkyl, and carbocyclic aralkyl, aralkenyl and aralkynyl groups. Such groups can be unsubstituted or substituted by one or more substituents as defined herein. The examples and preferences expressed below apply to each of the hydrocarbyl substituent groups or hydrocarbyl-containing substituent groups referred to in the various definitions of substituents for compounds of the formula (I) unless the context indicates otherwise.

Generally by way of example, the hydrocarbyl groups can have up to eight carbon atoms, unless the context requires otherwise. Within the sub-set of hydrocarbyl groups having 1 to 8 carbon atoms, particular examples are C₁₋₆ hydrocarbyl groups, such as C₁₋₄ hydrocarbyl groups (e.g. C₁₋₃ hydrocarbyl groups or C₁₋₂ hydrocarbyl groups), specific examples being any individual value or combination of values selected from C₁, C₂, C₃, C₄, C₅, C₆, C₇ and C₈ hydrocarbyl groups.

The term “alkyl” covers both straight chain and branched chain alkyl groups. Examples of alkyl groups include methyl, ethyl, propyl, isopropyl, n-butyl, isobutyl, tert-butyl, n-pentyl, 2-pentyl, 3-pentyl, 2-methyl butyl, 3-methyl butyl, and n-hexyl and its isomers. Within the sub-set of alkyl groups having 1 to 8 carbon atoms, particular examples are C₁₋₆ alkyl groups, such as C₁₋₄ alkyl groups (e.g. C₁₋₃ alkyl groups or C₁₋₂ alkyl groups).

Examples of cycloalkyl groups are those derived from cyclopropane, cyclobutane, cyclopentane, cyclohexane and cycloheptane. Within the sub-set of cycloalkyl groups the cycloalkyl group will have from 3 to 8 carbon atoms, particular examples being C₃₋₆ cycloalkyl groups.

Examples of alkenyl groups include, but are not limited to, ethenyl (vinyl), 1-propenyl, 2-propenyl (allyl), isopropenyl, butenyl, buta-1,4-dienyl, pentenyl, and hexenyl. Within the sub-set of alkenyl groups the alkenyl group will have 2 to 8 carbon atoms, particular examples being C₂₋₆ alkenyl groups, such as C₂₋₄ alkenyl groups.

Examples of cycloalkenyl groups include, but are not limited to, cyclopropenyl, cyclobutenyl, cyclopentenyl, cyclopentadienyl and cyclohexenyl. Within the sub-set of cycloalkenyl groups the cycloalkenyl groups have from 3 to 8 carbon atoms, and particular examples are C₃₋₆ cycloalkenyl groups.

Examples of alkynyl groups include, but are not limited to, ethynyl and 2-propynyl (propargyl) groups. Within the sub-set of alkynyl groups having 2 to 8 carbon atoms, particular examples are C₂₋₆ alkynyl groups, such as C₂₋₄ alkynyl groups.

Examples of carbocyclic aryl groups include substituted and unsubstituted phenyl groups.

Examples of cycloalkylalkyl, cycloalkenylalkyl, carbocyclic aralkyl, aralkenyl and aralkynyl groups include phenethyl, benzyl, styryl, phenylethynyl, cyclohexylmethyl, cyclopentylmethyl, cyclobutylmethyl, cyclopropylmethyl and cyclopentenylmethyl groups.

When present, a hydrocarbyl group can be optionally substituted by one or more substituents selected from hydroxy, oxo, alkoxy, carboxy, halogen, cyano, nitro, amino, mono- or di-C₁₋₄ hydrocarbylamino, and monocyclic or bicyclic carbocyclic and heterocyclic groups having from 3 to 12 (typically 3 to 10 and more usually 5 to 10) ring members. Preferred substituents include halogen such as fluorine. Thus, for example, the substituted hydrocarbyl group can be a partially fluorinated or perfluorinated group such as difluoromethyl or trifluoromethyl. In one embodiment preferred substituents include monocyclic carbocyclic and heterocyclic groups having 3-7 ring members.

One or more carbon atoms of a hydrocarbyl group may optionally be replaced by O, S, SO, SO₂, NR^{c}, X¹C(X²), C(X²)X¹ or X¹C(X²)X¹ wherein X¹ and X² are as hereinbefore defined. For example, 1, 2, 3 or 4 carbon atoms of the hydrocarbyl group may be replaced by one of the atoms or groups listed, and the replacing atoms or groups may be the same or different. Examples of groups in which a carbon atom of the hydrocarbyl group has been replaced by a replacement atom or group as defined above include ethers and thioethers (C replaced by O or S), amides, esters, thioamides and thioesters (C replaced by X¹C(X²) or C(X²)X¹), sulphones and sulphoxides (C replaced by SO or SO₂) and amines (C replaced by NR^{c}).

Where an amino group has two hydrocarbyl substituents, they may, together with the nitrogen atom to which they are attached, and optionally with another heteroatom such as nitrogen, sulphur, or oxygen, link to form a ring structure of 4 to 7 ring members.

The definition “R^{a}-R^{b}” as used herein, either with regard to substituents present on a carbocyclic or heterocyclic moiety, or with regard to other substituents present at other locations on the compounds of the formula (I), includes inter alia compounds wherein R^{a} is selected from a bond, O, CO, OC(O), SC(O), NR^{c}C(O), OC(S), SC(S), NR^{c}C(S), OC(NR^{c}), SC(NR^{c}), NR^{c}C(NR^{c}), C(O)O, C(O)S, C(O)NR^{c}, C(S)O, C(S)S, C(S)NR^{c}, C(NR^{c})O, C(NR^{c})S, C(NR^{c})NR^{c}, OC(O)O, SC(O)O, NR^{c}C(O)O, OC(S)O, SC(S)O, NR^{c}C(S)O, OC(NR^{c})O, SC(NR^{c})O, NR^{c}C(NR^{c})O, OC(O)S, SC(O)S, NR^{c}C(O)S, OC(S)S, SC(S)S, NR^{c}C(S)S, OC(NR^{c})S, SC(NR^{c})S, NR^{c}C(NR^{c})S, OC(O)NR^{c}, SC(O)NR^{c}, NR^{c}C(O)NR^{c}, OC(S)NR^{c}, SC(S)NR^{c}, NR^{c}C(S)NR^{c}, OC(NR^{c})NR^{c}, SC(NR^{c})NR^{c}, NR^{c}C(NR^{c}NR^{c}, S, SO, SO₂, NR^{c}, SO₂NR^{c} and NR^{c}SO₂ wherein R^{c} is as hereinbefore defined.

The moiety R^{b} can be hydrogen or it can be a group selected from carbocyclic and heterocyclic groups having from 3 to 12 ring members (typically 3 to 10 and more usually from 5 to 10), and a C₁₋₈ hydrocarbyl group optionally substituted as hereinbefore defined. Examples of hydrocarbyl, carbocyclic and heterocyclic groups are as set out above.

When R^{a} is O and R^{b} is a C₁₋₈ hydrocarbyl group, R^{a} and R^{b} together form a hydrocarbyloxy group. Preferred hydrocarbyloxy groups include saturated hydrocarbyloxy such as alkoxy (e.g. C₁₋₆ alkoxy, more usually C₁₋₄ alkoxy such as ethoxy and methoxy, particularly methoxy), cycloalkoxy (e.g. C₃₋₆ cycloalkoxy such as cyclopropyloxy, cyclobutyloxy, cyclopentyloxy and cyclohexyloxy) and cycloalkyalkoxy (e.g. C₃₋₆ cycloalkyl-C₁₋₂ alkoxy such as cyclopropylmethoxy).

The hydrocarbyloxy groups can be substituted by various substituents as defined herein. For example, the alkoxy groups can be substituted by halogen (e.g. as in difluoromethoxy and trifluoromethoxy), hydroxy (e.g. as in hydroxyethoxy), C₁₋₂ alkoxy (e.g. as in methoxyethoxy), hydroxy-C₁₋₂ alkyl (as in hydroxyethoxyethoxy) or a cyclic group (e.g. a cycloalkyl group or non-aromatic heterocyclic group as hereinbefore defined). Examples of alkoxy groups bearing a non-aromatic heterocyclic group as a substituent are those in which the heterocyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkoxy group is a C₁₋₄ alkoxy group, more typically a C₁₋₃ alkoxy group such as methoxy, ethoxy or n-propoxy.

Alkoxy groups substituted by a monocyclic group such as pyrrolidine, piperidine, morpholine and piperazine and N-substituted derivatives thereof such as N-benzyl, N—C₁₋₄ acyl and N—C₁₋₄ alkoxycarbonyl. Particular examples include pyrrolidinoethoxy, piperidinoethoxy and piperazinoethoxy.

When R^{a} is a bond and R^{b} is a C₁₋₈ hydrocarbyl group, examples of hydrocarbyl groups R^{a}-R^{b} are as hereinbefore defined. The hydrocarbyl groups may be saturated groups such as cycloalkyl and alkyl and particular examples of such groups include methyl, ethyl and cyclopropyl. The hydrocarbyl (e.g. alkyl) groups can be substituted by various groups and atoms as defined herein. Examples of substituted alkyl groups include alkyl groups substituted by one or more halogen atoms such as fluorine and chlorine (particular examples including bromoethyl, chloroethyl and trifluoromethyl), or hydroxy (e.g. hydroxymethyl and hydroxyethyl), C₁₋₈ acyloxy (e.g. acetoxymethyl and benzyloxymethyl), amino and mono- and dialkylamino (e.g. aminoethyl, methylaminoethyl, dimethylaminomethyl, dimethylaminoethyl and tert-butylaminomethyl), alkoxy (e.g. C₁₋₂ alkoxy such as methoxy—as in methoxyethyl), and cyclic groups such as cycloalkyl groups, aryl groups, heteroaryl groups and non-aromatic heterocyclic groups as hereinbefore defined).

Particular examples of alkyl groups substituted by a cyclic group are those wherein the cyclic group is a saturated cyclic amine such as morpholine, piperidine, pyrrolidine, piperazine, C₁₋₄-alkyl-piperazines, C₃₋₇-cycloalkyl-piperazines, tetrahydropyran or tetrahydrofuran and the alkyl group is a C₁₋₄ alkyl group, more typically a C₁₋₃ alkyl group such as methyl, ethyl or n-propyl. Specific examples of alkyl groups substituted by a cyclic group include pyrrolidinomethyl, pyrrolidinopropyl, morpholinomethyl, morpholinoethyl, morpholinopropyl, piperidinylmethyl, piperazinomethyl and N-substituted forms thereof as defined herein.

Particular examples of alkyl groups substituted by aryl groups and heteroaryl groups include benzyl and pyridylmethyl groups.

When R^{a} is SO₂NR^{c}, R^{b} can be, for example, hydrogen or an optionally substituted C₁₋₈ hydrocarbyl group, or a carbocyclic or heterocyclic group. Examples of R^{a}-R^{b} where R^{a} is SO₂NR^{c} include aminosulphonyl, C₁₋₄ alkylaminosulphonyl and di-C₁₋₄ alkylaminosulphonyl groups, and sulphonamides formed from a cyclic amino group such as piperidine, morpholine, pyrrolidine, or an optionally N-substituted piperazine such as N-methyl piperazine.

Examples of groups R^{a}-R^{b} where R^{a} is SO₂ include alkylsulphonyl, heteroarylsulphonyl and arylsulphonyl groups, particularly monocyclic aryl and heteroaryl sulphonyl groups. Particular examples include methylsulphonyl, phenylsulphonyl and toluenesulphonyl.

When R^{a} is NR^{c}, R^{b} can be, for example, hydrogen or an optionally substituted C₁₋₈ hydrocarbyl group, or a carbocyclic or heterocyclic group. Examples of R^{a}-R^{b} where R^{a} is NR^{c} include amino, C₁₋₄ alkylamino (e.g. methylamino, ethylamino, propylamino, isopropylamino, tert-butylamino), di-C₁₋₄ alkylamino (e.g. dimethylamino and diethylamino) and cycloalkylamino (e.g. cyclopropylamino, cyclopentylamino and cyclohexylamino).

The term upregulation of Aurora kinase as used herein is defined as including elevated expression or over-expression of Aurora kinase, including gene amplification (i.e. multiple gene copies) and increased expression by a transcriptional effect, and hyperactivity and activation of Aurora kinase, including activation by mutations.

### SPECIFIC EMBODIMENTS AND PREFERENCES

The following preferences, embodiments and examples apply to compounds of the formulae (I), (Ia), (Ib) and (Ic).

R⁰ can be hydrogen or, together with the group R^{g} when present, can form a bridging group —(CH₂)ₚ— wherein p is 2 to 4, more usually 2-3, e.g. 2. Preferably R⁰ is hydrogen.

When R⁰ and the group R^{g} form a bridging group —(CH₂)ₚ—, the entity —(CH₂)ₘ—(B)ₙ—NR⁰— can be represented thus: 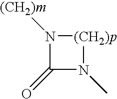
A is a bond or —(CH₂)ₘ—(B)ₙ— wherein B is C═O, NR^{g}(C═O) or O(C═O), m is 0, 1 or 2; and n is 0 or 1. In one preferred group of compounds of the invention, m is 0 or 1, n is 1 and B is C═O or NR^{g}(C═O), preferably C═O. More preferably, m is 0, n is 1 and B is C═O. It is presently preferred that when B is NR^{g}(C═O), R^{g} is hydrogen.

It will be appreciated that the moiety R¹-A-NH linked to the 4-position of the pyrazole ring can take the form of an amine R¹—(CH₂)ₘ—NH, an amide R¹—(CH₂)ₘ—C(═O)NH, a urea R¹—(CH₂)ₘ—NHC(═O)NH or a carbamate R¹—(CH₂)ₘ—OC(═O)NH wherein in each case m is 0, 1 or 2, preferably 0 or 1 and most preferably 0.

Compounds of the Formula (I) Wherein R^{q} is a Group (a) or (b), and Compounds of the Formulae (Ia) and (Ib)

The following preferences, embodiments and examples apply to X, R¹ to R⁶ and R¹⁰ in formula (I) where R^{q} is a group (a) or (b), and in formulae (Ia) and (Ib).

X can be N or CR⁵. In one particular embodiment, X is CR⁵. In another particular embodiment, X is N. Preferably X is CH. Preferably compounds of the invention are of formula (Ia), in particular where X is CR⁵.

R¹ is hydrogen, a carbocyclic or heterocyclic group having from 3 to 12 ring members, or an optionally substituted C₁₋₈ hydrocarbyl group as hereinbefore defined. Examples of carbocyclic or heterocyclic groups and optionally substituted hydrocarbyl groups and general preferences for such groups are as set out above.

In one embodiment, R¹ is hydrogen, a carbocyclic or heterocyclic group having from 3 to 12 ring members, or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂.

In one embodiment, R¹ is an aryl or heteroaryl group.

When R¹ is a heteroaryl group, particular heteroaryl groups include monocyclic heteroaryl groups containing up to three heteroatom ring members selected from O, S and N, and bicyclic heteroaryl groups containing up to 2 heteroatom ring members selected from O, S and N and wherein both rings are aromatic. The heteroaryl groups may be unsubstituted or substituted by one or more substituent groups as hereinbefore defined.

Particular examples of R¹ include heteroaryl groups selected from pyrazolopyridinyl (e.g. pyrazolo[1,5-a]pyridin-3-yl), cinnoline, benzoisoxazole, furanyl (e.g. 2-furanyl and 3-furanyl), indolyl (e.g. 3-indolyl, 4-indolyl and 7-indolyl), oxazolyl, thiazolyl (e.g. thiazol-2-yl and thiazol-5-yl), isoxazolyl (e.g. isoxazol-3-yl and isoxazol-4-yl), pyrrolyl (e.g. 3-pyrrolyl), pyridyl (e.g. 2-pyridyl), quinolinyl (e.g. quinolin-8-yl), 2,3-dihydro-benzo[1,4]dioxine (e.g. 2,3-dihydro-benzo[1,4]dioxin-5-yl), benzo[1,3]dioxole (e.g. benzo[1,3]dioxol-4-yl), 2,3-dihydrobenzofuranyl (e.g. 2,3-dihydrobenzofuran-7-yl), imidazolyl and thiophenyl (e.g. 3-thiophenyl).

In one embodiment, R¹ is a bicyclic hetereroaryl group whereby the bicyclic group may contain two aromatic rings or an aromatic ring and a non-aromatic ring. Presently preferred R¹ heteroaryl groups include cinnoline, benzoisoxazole, 2,3-dihydrobenzofuranyl (e.g. 2,3-dihydrobenzofuran-7-yl), and pyrazolopyridine (e.g. pyrazolo[1,5-a]pyridine).

In one sub-group of compounds, R¹ is a bicyclic hetereroaryl group containing 2 heteroatoms independently selected from O and N and wherein both rings are aromatic. Typically, at least one of the heteroatoms will be N. Preferred groups are a pyrazolo[1,5-a]pyridine group, such as a 3-pyrazolo[1,5-a]pyridinyl group, a cinnoline group such as cinnolin-4-yl and benzoisoxazole group such as benzo[c]isoxazol-3-yl.

In another subgroup of compounds, R¹ is a bicyclic hetereroaryl group whereby there is a phenyl ring with a non-aromatic heterocyclic group is fused to it. Preferred fused rings include oxa-, dioxa-, aza-, diaza- or oxa-aza-cycloalkyl groups. Preferably they form a cyclic group selected from those below.

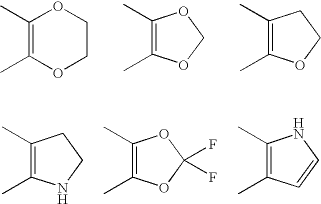

Typically the fused cycloalkyl group will contain an oxygen. Preferably the fused ring will be an oxa- or dioxa-cycloalkyl group such as one of those outlined below.

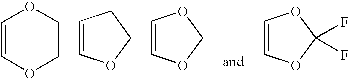

A particular example is 2,3-dihydrobenzofuranyl (e.g. 2,3-dihydrobenzofuran-7-yl).

In another embodiment, the group R¹ is a five membered heteroaryl group containing 1 or 2 ring heteroatoms selected from O, N and S. Particular five membered heteroaryl groups include furan, thiophene, pyrrole, oxazole, isoxazole and thiazole groups. Particularly preferred five membered heteroaryl group contain an oxygen for example furan. The heteroaryl groups may be unsubstituted or substituted by one or more substituent groups as hereinbefore defined. A particular example is a 2,3 disubstituted furan-5-yl.

A preferred R¹ aryl group is a phenyl ring.

Preferred non-aromatic groups R¹ include monocyclic cycloalkyl such as cyclopropyl, cyclohexyl, cyclopentyl, oxacycloalkyl such as tetrahydropyran and tetrahydrofuran and azacycloalkyl groups such as piperidinyl, particularly cyclopropyl, cyclohexyl, tetrahydropyran and 4-piperidinyl groups

One sub-group of preferred non-aromatic groups R¹ consists of monocyclic cycloalkyl such as cyclopropyl, cyclohexyl, cyclopentyl, oxacycloalkyl such as tetrahydropyran and azacycloalkyl groups such as piperidinyl, particularly cyclopropyl, cyclohexyl, tetrahydropyran and 4-piperidinyl groups.

Particular examples of non-aromatic R¹ groups include unsubstituted or substituted (by one or more groups R¹⁰) monocyclic cycloalkyl groups such as cyclopropyl, cyclobutyl, cyclopentyl, cyclohexyl and cycloheptyl, particularly cyclopropyl, and unsubstituted or substituted (by one or more groups R¹⁰) 5-, 6- and 7-membered monocyclic heterocyclic groups such as tetrahydropyran, morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, piperazine, and N-alkyl piperazines such as N-methyl piperazine. In general, a preferred non-aromatic heterocyclic R¹ group is tetrahydropyran. Another particular non-aromatic R₁ group is tetrahydrofuran, for example a 2-tetrahydrofuranyl group.

When R¹ is a C₁₋₈ hydrocarbyl group substituted by a carbocyclic or heterocyclic group, the carbocyclic and heterocyclic groups can be aromatic or non-aromatic and can be selected from the examples of such groups set out hereinabove.

When the carbocyclic or heterocylic group attached to the a C₁₋₈ hydrocarbyl group is aromatic, examples of such groups include monocyclic aryl groups and monocyclic heteroaryl groups containing up to four heteroatom ring members selected from O, S and N, and bicyclic heteroaryl groups containing up to 2 heteroatom ring members selected from O, S and N and wherein both rings are aromatic. Examples of such groups include furanyl (e.g. 2-furanyl or 3-furanyl), indolyl, oxazolyl, isoxazolyl, pyridyl, quinolinyl, pyrrolyl, imidazolyl and thienyl. Particular examples of aryl and heteroaryl groups as substituents for a C₁₋₈ hydrocarbyl group include phenyl, imidazolyl, tetrazolyl, triazolyl, indolyl, 2-furanyl, 3-furanyl, pyrrolyl and thienyl.

When R¹ is a C₁₋₈ hydrocarbyl group substituted by a non-aromatic carbocyclic or heterocyclic group, the non-aromatic or heterocyclic group may be a group selected from the lists of such groups set out hereinabove. For example, the non-aromatic group can be a monocyclic group having from 5 to 7 ring members and typically containing from 0 to 3, more typically 0, 1 or 2, heteroatom ring members selected from O, S and N. Particular examples include monocyclic cycloalkyl groups such as cyclopropyl, cyclobutyl, cyclopentyl, cyclohexyl and cycloheptyl, and 5-, 6- and 7-membered monocyclic heterocyclic groups such as tetrahydropyran, morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, piperazine, and N-alkyl piperazines such as N-methyl piperazine. In general, preferred non-aromatic heterocyclic groups include tetrahydropyran, pyrrolidine, piperidine, morpholine, thiomorpholine and N-methyl piperazine.

When R¹ is an optionally substituted C₁₋₈ hydrocarbyl group, the hydrocarbyl group may be as hereinbefore defined, and is preferably up to four carbon atoms in length, more usually up to three carbon atoms in length for example one or two carbon atoms in length. In one embodiment, the hydrocarbyl group is a linear saturated group having from 1-6 carbon atoms, more usually 1-4 carbon atoms, for example 1-3 carbon atoms, e.g. 1, 2 or 3 carbon atoms. When the hydrocarbyl group is substituted, particular examples of such groups are substituted (e.g. by a carbocyclic such as phenyl or a heterocyclic group) methyl and ethyl groups. A preferred substituted C₁₋₈ hydrocarbyl R¹ group is aralkyl groups such as phenyl. Another preferred substituted C₁₋₈ hydrocarbyl R¹ group is methyl substituted by a 6 membered non-aromatic heterocycle such as tetrahydropyran.

Preferred substituted and unsubstituted C₁₋₈ hydrocarbyl groups include trifluoromethyl and tertiary butyl groups.

Particularly preferred R¹ groups are phenyl groups.

The group R¹ can be an unsubstituted or substituted carbocyclic or heterocyclic group in which one or more substituents can be selected from the group R¹⁰ as hereinbefore defined. In one embodiment, the substituents on R¹ may be selected from the group R^{10a} consisting of halogen, hydroxy, trifluoromethyl, cyano, nitro, carboxy, heterocyclic groups having 5 or 6 ring members and up to 2 heteroatoms selected from O, N and S, a group R^{a}-R^{b} wherein R^{a} is a bond, O, CO, X³C(X⁴), C(X⁴)X³, X³C(X⁴)X³, S, SO, or SO₂, and R^{b} is selected from hydrogen, heterocyclic groups having 5 or 6 ring members and up to 2 heteroatoms selected from O, N and S, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In one embodiment R¹ is an unsubstituted carbocyclic or heterocyclic group.

In a further embodiment, the substituents on R¹ may be selected from the group R^{10b} consisting of halogen, hydroxy, trifluoromethyl, cyano, nitro, carboxy, a group R^{a}-R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In another embodiment, the substituents on R¹ may be selected from halogen, hydroxy, trifluoromethyl, a group R^{a}-R^{b} wherein R^{a} is a bond or O, and R^{b} is selected from hydrogen and a C₁₋₄ hydrocarbyl group optionally substituted by one or more substituents selected from hydroxyl and halogen (preferably fluorine).

Particular examples of substituents that may be present on a group R¹ (e.g. an aryl or heteroaryl group R¹) include fluorine, chlorine, methoxy, methyl, oxazolyl, morpholino, trifluoromethyl, bromomethyl, chloroethyl, pyrrolidino, pyrrolidinylethoxy, pyrrolidinylmethyl, difluoromethoxy and morpholinomethyl.

The moiety R¹ may be substituted by more than one substituent. Thus, for example, there may be 1 or 2 or 3 or 4 substituents, more typically 1, 2 or 3 substituents. In one embodiment, where R¹ is a six membered ring (e.g. a carbocyclic ring such as a phenyl ring), there may be a single substituent which may be located at any one of the 2-, 3- and 4-positions on the ring. In another embodiment, there may be two or three substituents and these may be located at the 2-, 3-, 4-, 5- or 6-positions around the ring.

By way of example, a phenyl group R¹ may be 2-monosubstituted, 3-monosubstituted, 2,6-disubstituted, 2,3-disubstituted, 2,4-disubstituted, 2,5-disubstituted, 2,3,6-trisubstituted or 2,4,6-trisubstituted. More particularly, a phenyl group R¹ may be be monosubstituted at the 2-position or disubstituted at positions 2- and 6- with substituents selected from fluorine, chlorine and R^{a}-R^{b}, where R^{a} is O and R^{b} is C₁₋₄ alkyl (e.g. methyl or ethyl), with fluorine, chlorine and methoxy being particularly preferred substituents.

In another group of preferred compounds, the phenyl group R¹ is 2,4-disubstituted or 2,5-disubstituted. The 2-substituent may be, for example, a halogen (e.g. F or Cl) or a methoxy group. In one particular group of compounds, the 2-substituent is methoxy. The 5-substituent, when present, can be selected from, for example, halogen (e.g. Cl or F), C₁₋₄ alkyl (e.g. tert-butyl or isopropyl), methoxy, trifluoromethoxy, trifluoromethyl, or a group HetN-SO₂— where “HetN” is a nitrogen-containing saturated monocyclic heterocycle such as piperazino, N—C₁₋₄ alkylpiperazino, morpholino, piperidino or pyrrolidino. One preferred 5-substitutent is Cl, and a preferred 2,5-combination is 2-methoxy-5-chlorophenyl.

In a further group of compounds, the phenyl group R¹ has a single substituent at the 4-position of the phenyl ring. The substituent can be, for example, a halogen atom (preferably fluorine or chlorine, most preferably fluorine) or a trifluoromethyl group.

Particular examples of groups R¹ include the groups A1 to A69 set out in Table 1 below.

TABLE 1

| | |
|---|---|
| 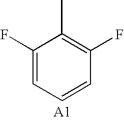 | 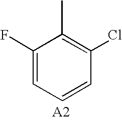 |
| 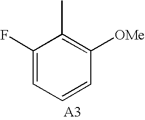 | 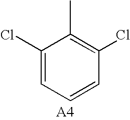 |
| 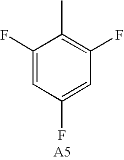 | 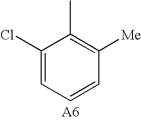 |
| 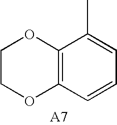 | 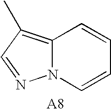 |
| 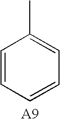 | 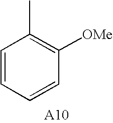 |
| 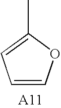 | 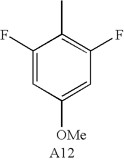 |
| 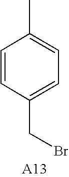 | 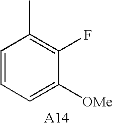 |
| 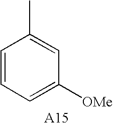 | 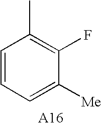 |
| 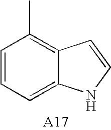 | 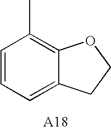 |
| 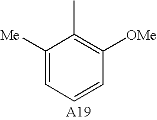 | 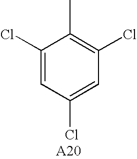 |
| 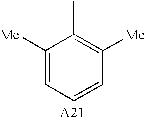 | 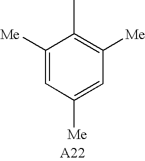 |
| 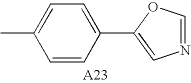 | 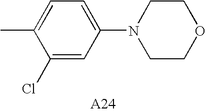 |
| 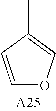 | 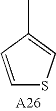 |
| 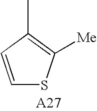 | 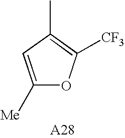 |
| 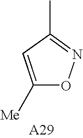 | 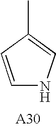 |
| 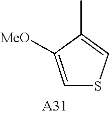 | 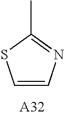 |
| 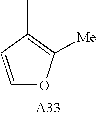 | 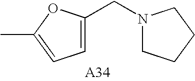 |
| 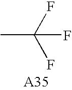 | 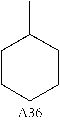 |
| 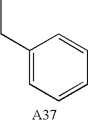 | 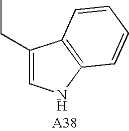 |
| 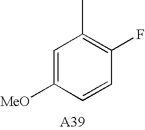 | 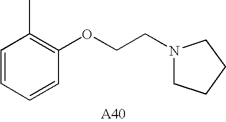 |
| 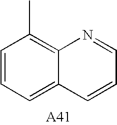 | 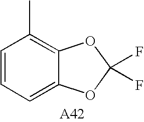 |
| 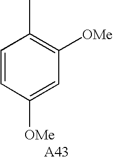 | 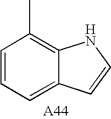 |
| 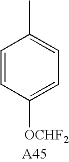 | 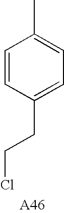 |
| 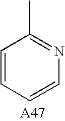 | 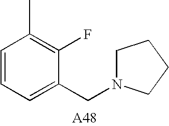 |
| 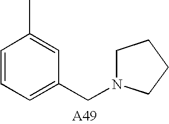 | 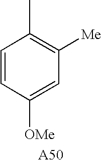 |
| 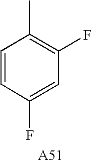 | 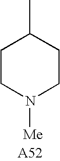 |
| 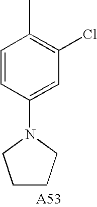 | 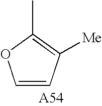 |
| 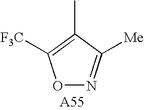 | 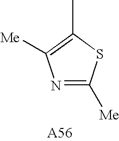 |
| 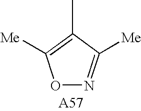 | 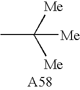 |
| 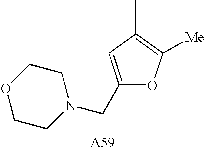 | 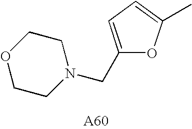 |
| 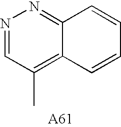 | 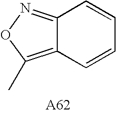 |
| 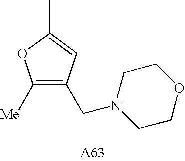 | 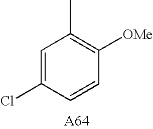 |
| 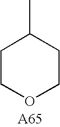 | 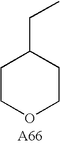 |
| 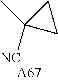 | 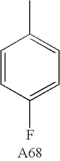 |
| 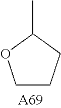 | |

Preferred groups R¹ include groups A1 to A10, A18, A56, A59, A60, A61, A62 and A63-A68. Typically R¹ is selected from A1, A56, A59, A63, A64, A65, A66, A67 and A68.

Particularly preferred groups R¹ include 2,6-difluorophenyl, 2-methoxy-5-chloro-phenyl, tetrahydropyran, 4-(2-methyl-5-furan-3-ylmethyl)-morpholine, and 4-methyltetrahydropyran.

A currently most preferred group R¹ is 2,6-difluorophenyl or 4-(2-methyl-5-furan-3-ylmethyl)-morpholine.

R² is hydrogen, halogen, methoxy, or a C₁₋₄ hydrocarbyl group optionally substituted by halogen, hydroxyl or methoxy. Preferably R² is hydrogen, chlorine or methyl, and most preferably R² is hydrogen.

The moieties R³, R⁵ and R⁶ are typically selected from hydrogen, halogen, hydroxy, trifluoromethyl, cyano, nitro, carboxy, amino, monocyclic carbocyclic and heterocyclic groups having from 3 to 12 (preferably 3 to 7, and more typically 5 or 6) ring members, a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, a carbocyclic or heterocyclic group with 3-7 ring members and a C₁₋₈ hydrocarbyl group optionally substituted by one or more substituents selected from hydroxy, C₁₋₄ acyloxy, oxo, halogen, cyano, nitro, carboxy, amino, mono- or di-C₁₋₄ hydrocarbylamino, a carbocyclic or heterocyclic group with 3-7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; and R^{c}, X¹ and X².

In one embodiment, R³, R⁵ and R⁶ are each hydrogen or are selected from halogen, cyano, hydroxy, trifluoromethyl, nitro, a group R^{a}-R^{b} wherein R^{a} is a bond, O, CO or C(X²)X¹ and R^{b} is selected from hydrogen, heterocyclic groups having from 3 to 12 ring members (preferably 4 to 7 ring members), and a C₁₋₈ hydrocarbyl group (preferably a C₁₋₄ hydrocarbyl group), optionally substituted by one or more substituents selected from hydroxy, C₁₋₄ acyloxy, mono- or di-C₁₋₄ hydrocarbylamino, heterocyclic groups having from 3 to 12 ring members, more preferably 4 to 7 ring members; where R^{c} is selected from hydrogen and C₁₋₄ hydrocarbyl, X¹ is O or NR^{c} and X² is ═O.

In another embodiment, R³, R⁵ and R⁶ are selected from hydrogen, fluorine, chlorine, bromine, nitro, trifluoromethyl, carboxy, a group R^{a}-R^{b} wherein R^{a} is a bond, O, CO, C(X²)X¹, and R^{b} is selected from hydrogen, heterocyclic groups having 3-7 ring members (e.g. pyrrolidine, N-methyl piperazine or morpholine) and a C₁₋₄ hydrocarbyl group optionally substituted by one or more substituents selected from hydroxy, carboxy, C₁₋₄ acyloxy, amino, mono- or di-C₁₋₄ hydrocarbylamino, heterocyclic groups with 3-7 ring members (e.g. pyrrolidine, N-methyl piperazine or morpholine); or an adjacent pair of substituents selected from R³, R⁴, R⁵ and R⁶ together with the carbon atoms to which they are attached may form a non-aromatic five or six membered ring containing one or two oxygen atoms as ring members.

In a more preferred embodiment, R³, R⁵ and R⁶ are selected from hydrogen, fluorine, chlorine, trifluoromethyl, a group R^{a}-R^{b} wherein R^{a} is a bond, O, CO, C(X²)X¹, and R^{b} is selected from hydrogen, saturated heterocyclic groups having 5-6 ring members and a C₁₋₂ hydrocarbyl group optionally substituted by one or more substituents selected from hydroxy, carboxy, C₁₋₂ acyloxy, amino, mono- or di-C₁₋₄ hydrocarbylamino, heterocyclic groups with 5-6 ring members; or an adjacent pair of substituents selected from R³, R⁴, R⁵ and R⁶ may form a methylenedioxy or ethylenedioxy group each optionally substituted by one or more fluorine atoms.

In another embodiment, particular substituent groups R³, R⁵ and R⁶ include hydrogen, halogen, a group R^{a}-R^{b} wherein R^{a} is a bond, O, CO, C(X²)X¹, and R^{b} is selected from hydrogen, heterocyclic group having 3-7 ring members and a C₁₋₄ hydrocarbyl group optionally substituted by one or more substituents selected from hydroxy, carboxy, amino, mono- or di-C₁₋₄ hydrocarbylamino, heterocyclic group with 3-7 ring members.

Typically R³, R⁵ and R⁶ are all hydrogen.

The group R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

Preferably R⁴ is selected from hydrogen and a group R^{d}-R^{e} wherein R^{d} is a bond, CO, C(X²)X¹, or SO₂; and R^{e} is selected from carbocyclic and heterocyclic groups having from 3 to 7 ring members, and a C₁₋₄ hydrocarbyl group optionally substituted by one or more substituents selected from hydroxy, alkoxy, halogen, amino, mono- or di-C₁₋₄ hydrocarbylamino, carbocyclic and heterocyclic groups having from 3 to 7 ring members.

In one embodiment R⁴ is selected from hydrogen and a group R^{d}-R^{e} wherein R^{d} is a bond and R^{e} is a C₁₋₄ hydrocarbyl group optionally substituted by one or more substituents selected from hydroxy, alkoxy, halogen preferably fluorine, carbocyclic and heterocyclic groups having from 3 to 7 ring members.

In another embodiment, R⁴ is selected from hydrogen and unsubstituted or substituted C₁₋₄ alkyl groups where the substituents are heterocyclic groups having 3-7 ring members preferably a 5-6-membered non-aromatic heterocycle.

In another embodiment, R⁴ is selected from hydrogen and unsubstituted or substituted C₁₋₄ alkyl groups where the substituents are carbocyclic groups having 3-7 ring members preferably a 6-membered carbocycle. Most preferably R⁴ groups are substituted C₁₋₄ alkyl group where the substituents are aromatic carbocyclic groups. Preferred R⁴ groups are aralkyl groups such as benzyl.

Preferably R⁴ is hydrogen, or methyl, ethyl or propyl optionally substituted with an unsubstituted 6-membered non-aromatic heterocycle such as N-alkyl-piperidine, morpholine, tetrahydropyran or an unsubstituted 6-membered carbocycle such as phenyl. Particularly preferred R⁴ groups are hydrogen, benzyl, methyl, 4-methyl-N-methyl-piperidine, 2-morpholin-4-yl-ethyl, 3-morpholin-4-yl-propyl, tetrahydropyran-4-yl-methyl. Typically R⁴ is hydrogen, methyl or benzyl.

In one embodiment it is preferred that R⁴ is a substituent as defined herein other than hydrogen.

In another embodiment group R⁴ is selected from trifluoromethyl, carboxy, carbocyclic and heterocyclic groups having from 3 to 12 ring members; a group R^{d}—R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

Preferably R⁴ is selected from a group R^{d}-R^{e} wherein R^{d} is a bond, CO, C(X²)X¹, or SO₂; and R^{e} is selected from carbocyclic and heterocyclic groups having from 3 to 7 ring members, and a C₁₋₄ hydrocarbyl group optionally substituted by one or more substituents selected from hydroxy, alkoxy, halogen, amino, mono- or di-C₁₋₄ hydrocarbylamino, carbocyclic and heterocyclic groups having from 3 to 7 ring members.

In one embodiment R⁴ is selected from a group R^{d}-R^{e} wherein R^{d} is a bond and R^{e} is a C₁₋₄ hydrocarbyl group optionally substituted by one or more substituents selected from hydroxy, alkoxy, halogen preferably fluorine, carbocyclic and heterocyclic groups having from 3 to 7 ring members.

In another embodiment, R⁴ groups are selected from unsubstituted or substituted C₁₋₄ alkyl group where the substituents are heterocyclic groups having 3-7 ring members preferably a 5-6-membered non-aromatic heterocycle.

In another embodiment, R⁴ groups are selected from unsubstituted or substituted C₁₋₄ alkyl group where the substituents are carbocyclic groups having 3-7 ring members preferably a 6-membered carbocycle. Most preferably R⁴ groups are substituted C₁₋₄ alkyl group where the substituents are aromatic carbocyclic groups. Preferred R⁴ groups are aralkyl groups such as benzyl.

Preferably R⁴ is methyl, ethyl or propyl optionally substituted with an unsubstituted 6-membered non-aromatic heterocycle such as N-alkyl-piperidine, morpholine, tetrahydropyran or an unsubstituted 6-membered carbocycle such as phenyl. Particularly preferred R⁴ groups are methyl, benzyl, N-methyl-piperidin-4-yl-methyl, 2-morpholin-4-yl-ethyl, 3-morpholin-4-yl-propyl, or tetrahydro-pyran-4-ylmethyl.

Whereas, in compounds of formula (Ia), each of R⁵ (where present) and R⁶ can be hydrogen or a substituent as hereinbefore defined other than hydrogen, it is preferred that at least one, more preferably both of R⁵ and R⁶ are hydrogen.

In one particular embodiment, one of R⁵ and R⁶ is a substituent as hereinbefore defined other than hydrogen and the other is hydrogen. For example, R⁵ can be a substituent other than hydrogen and R⁶ is hydrogen, or R⁶ can be a substituent other than hydrogen and R⁵ is hydrogen.

In another particular embodiment, both of R⁵ and R⁶ are other than hydrogen. Whereas, in compounds of formula (Ib), each of R³ and R⁶ can be hydrogen or a substituent as hereinbefore defined other than hydrogen, it is preferred that at least one, more preferably both, of R³ and R⁶ are hydrogen.

In one particular embodiment, one of R³ and R⁶ is a substituent as hereinbefore defined other than hydrogen and the other is hydrogen. For example, R³ can be a substituent other than hydrogen and R⁶ can be hydrogen, or R⁶ can be a substituent other than hydrogen and R³ can be hydrogen.

In another particular embodiment, both of R³ and R⁶ are other than hydrogen.

In a further embodiment, in compounds of formulas (Ia) and (Ib) it is preferred that R⁴ is a substitutent as defined herein other than hydrogen and that at least one, or two of R³ (where present), R⁵ (where present), or R⁶ are hydrogen.

In one particular embodiment for compounds of formulas (Ia), R⁴ is other than hydrogen and one of R⁵ (where present) or R⁶ is other than hydrogen and the other is hydrogen. For example, R⁴ and R⁶ are other than hydrogen and R⁵ is hydrogen, or R⁵ and R⁴ can be other than hydrogen and R⁶ is hydrogen.

In another particular embodiment, R⁴ is other than hydrogen and both of R⁵ and R⁶ are other than hydrogen.

In one particular embodiment for compounds of formula (Ib), R⁴ is other than hydrogen and one of R³ or R⁶ is other than hydrogen and the other is hydrogen. For example, R⁴ and R⁶ are other than hydrogen and R³ is hydrogen, or R³ and R⁴ can be other than hydrogen and R⁶ is hydrogen.

In another particular embodiment, R⁴ is other than hydrogen and both of R³ and R⁶ are other than hydrogen.

In one particularly preferred embodiment for compounds of formula (Ia) R⁶ is hydrogen and R⁴ and R⁵ (where present) is other than hydrogen.

R³ is preferably selected from:

hydrogen;

halogen (preferably fluorine or chlorine);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R⁴ is preferably selected from:

C₁₋₄ alkyl (for example methyl, ethyl or propyl) optionally substituted by a unsubstituted 6-membered non-aromatic heterocycle (such as N-alkyl-piperidine, morpholine, tetrahydropyran) or an unsubstituted 6-membered carbocycle (such as phenyl).

R⁵ is preferably selected from:

hydrogen;

halogen (preferably fluorine or chlorine);

C₁₋₄ alkoxy (for example methoxy);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R⁶ is preferably selected from hydrogen, fluorine and methyl, most preferably hydrogen.

In the foregoing definitions, when R¹¹ and R¹² together with the nitrogen atom in the group NR¹¹R¹² form a five or six membered heterocyclic ring, the heteroatom ring members are preferably selected from O and N. The heterocyclic ring is typically non-aromatic and examples of such rings include morpholine, piperazine, N—C₁₋₄-alkylpiperazine, piperidine and pyrrolidine. Particular examples of N—C₁₋₄-alkylpiperazine groups include N-methylpiperazine and N-isopropylpiperazine.

Preferred groups X, and R⁴ to R⁶ groups in formula (Ia) include those in which the 4-oxo-4,5-dihydro-1H-imidazo[4,5-c]pyridin-2-yl and 4-oxo-4,5-dihydro-1H-imidazo[4,5-d]pyridazin-2-yl groups

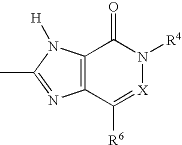

are as shown in Table 2 below.

TABLE 2

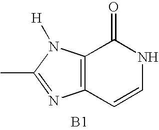

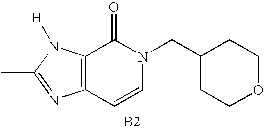

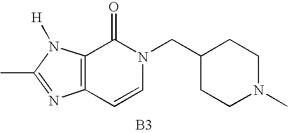

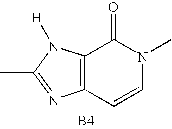

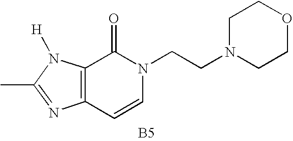

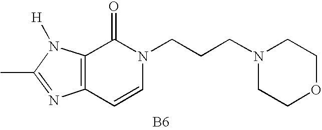

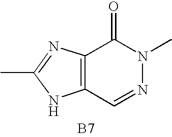

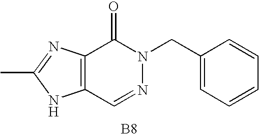

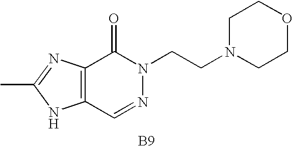

Of the 4-oxo-4,5-dihydro-1H-imidazo[4,5-c]pyridin-2-yl and 4-oxo-4,5-dihydro-1H-imidazo[4,5-d]pyridazin-2-yl groups set out in Table 2 above, particular groups include groups B1, B7, and B8.

One preferred group of compounds of the invention within formula (Ia) is represented by the formula (II): 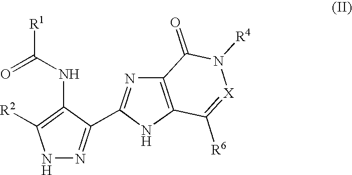
wherein X, R¹, R² and R⁴ to R⁶ are independently selected from X, R¹, R² and R⁴ to R⁶ or subgroups thereof as hereindefined.

Within formula (II), it is preferred that R² is hydrogen or C₁₋₄ alkyl, and more typically R² is hydrogen.

Within the group of compounds defined by the formula (II), R¹ is preferably 2-substituted, 2,6 disubstituted or 2,4,6, trisubstituted phenyl or a bicyclic heteraryl group, where the substituents are selected from halogen and C₁₋₄ alkoxy.

More preferably R¹ is selected from 2-methoxyphenyl, 2,6-difluorophenyl, 2-fluoro-6-methoxyphenyl, 2,6-dichlorophenyl, 2,4,6-trifluorophenyl, cinnoline (e.g. cinnolin-4-yl), benzoisoxazole (e.g. such as benzo[c]isoxazol-3-yl.), 2,3-dihydrobenzofuranyl (e.g. 2,3-dihydrobenzofuran-7-yl), and pyrazolopyridine (e.g. pyrazolo[1,5-a]pyridine group, such as a 3-pyrazolo[1,5-a]pyridinyl group).

One particularly preferred group R¹ is 2,6-difluorophenyl.

Within Formula (II), it is preferred that R³, R⁵ and R⁶ is hydrogen, halogen, a group R^{a}-R^{b} wherein R^{a} is a bond, O, CO, C(X²)X¹, and R^{b} is selected from hydrogen, heterocyclic group having 3-7 ring members and a C₁₋₄ hydrocarbyl group optionally substituted by one or more substituents selected from hydroxy, carboxy, amino, mono- or di-C₁₋₄ hydrocarbylamino, heterocyclic group with 3-7 ring members.

More preferably R³, R⁵ and R⁶ are hydrogen.

It is preferred that R⁴ groups are selected from hydrogen and unsubstituted or substituted C₁₋₄ alkyl group where the substituents are heterocyclic groups having 3-7 ring members preferably a 5-6-membered non-aromatic heterocycles or carbocycles having 6-members, preferably aromatic carbocycles.

Preferably R⁴ is hydrogen, or methyl, ethyl or propyl optionally substituted with an unsubstituted 6-membered non-aromatic heterocycle such as N-alkyl-piperidine, morpholine, tetrahydropyran or an unsubstituted 6-membered carbocycle such as phenyl. Particularly preferred R⁴ groups are hydrogen, benzyl, methyl, 4-methyl-N-methyl-piperidine, 2-ethyl-morpholine, 3-propyl-morpholine, or methyl-tetrahydro-pyran. Typically R⁴ is hydrogen, methyl or benzyl.

It is even more preferred that R⁴ is selected from unsubstituted and substituted C₁₋₄ alkyl group where the substituents are heterocyclic or carbocyclic groups having 3-7 ring members preferably a 5-6-membered non-aromatic heterocycle or an 6-membered carbocycle, for example methyl, ethyl or propyl optionally substituted with an unsubstituted 6-membered non-aromatic heterocycle such as N-alkyl-piperidine, morpholine, tetrahydropyran or an unsubstituted 6-membered carbocycle such as phenyl. Particularly preferred R⁴ groups are methyl, benzyl, 4-methyl-N-methyl-piperidine, 2-ethyl-morpholine, 3-propyl-morpholine, or methyl-tetrahydro-pyran.

Another preferred group of compounds of the invention within formula (Ia) is represented by the formula (III): 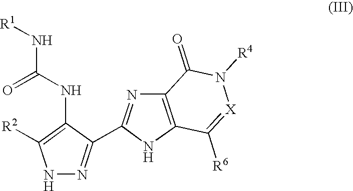
wherein X, R¹, R² and R⁴ to R⁶ are independently selected from X, R¹, R² and R⁴ to R⁶ or subgroups thereof as hereindefined.

The following embodiments may also include salts or solvates or N-oxides or esters or isomers thereof.

In one embodiment, when R⁴ is hydrogen it is preferred that R¹ is an optionally substituted C₁₋₈ hydrocarbyl group

In another embodiment, when R⁴ is hydrogen it is preferred that where R¹ is a carbocyclic or heterocyclic group having from 3 to 12 ring members it is not substituted with a bicyclic heteroaryl containing a phenyl ring fused to a S(O)₂ containing heterocycle.

In another embodiment, when R⁴ is hydrogen it is preferred that where R¹ is a carbocyclic or heterocyclic group having from 3 to 12 ring members it is not substituted with amino, mono- or di-C₁₋₄ hydrocarbylamino, a 3 to 10 ring members carbocycle, a 5 to 10 ring members heterocycle, or a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from carbocyclic and heterocyclic groups having from 3 to 12 ring members and a C₁₋₈ hydrocarbyl group substituted by one or more substituents selected from amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.
R^{c} is selected from hydrogen and C₁₋₄ hydrocarbyl; and
X¹ is O, S or NR^{c} and X² is ═O, ═S or ═NR^{c}.

In another embodiment, when R⁴ is hydrogen it is preferred that where R¹ is a carbocyclic or heterocyclic group having from 3 to 12 ring members it is substituted by one or more substituent groups selected from halogen, hydroxy, trifluoromethyl, cyano, nitro, carboxy and a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, and a C₁₋₈ hydrocarbyl group optionally substituted by one or more substituents selected from hydroxy, oxo, halogen, cyano, nitro, carboxy.
R^{c} is selected from hydrogen and C₁₋₄ hydrocarbyl; and
X¹ is O, S or NR^{c} and X² is ═O, ═S or ═NR^{c}.

It is preferred that R³, R⁴, R⁵ or R⁶ does not contain a quarternary amine either directly linked to the 4-oxo-4,5-dihydro-1H-imidazo[4,5-c]pyridin-2-yl, 4-oxo-4,5-dihydro-1H-imidazo[4,5-d]pyridazin-2-yl or 6-oxo-5,6-dihydro-1H-imidazo[4,5-c]pyridin-2-yl rings or linked via a —CH₂— group.

In one embodiment, when R¹ is an aromatic carbocyclic or heterocyclic group it is preferred that R³, R⁴, R⁵ or R⁶ are not —(NR^{x})C(═O)—C₃₋₆ cycloalkyl, —(NR^{x})C(═O)—C₃₋₆ cycloalkenyl, where R^{x} is selected from hydrogen and C₁₋₆ alkyl where the C₃₋₆ cycloalkyl or C₃₋₆ cycloalkenyl group may be optionally substituted.

In another embodiment, when R¹ is an optionally substituted C₃₋₆ cycloalkyl or C₃₋₆ cycloalkenyl, it is preferred that R³, R⁴, R⁵ or R⁶ are not —(NR^{x})—Y— (CH₂)ₙ—W where R^{x} is selected from hydrogen and C₁₋₆ alkyl, Y is a bond, C(O), —C(O)O, C(O)NR^{x}, C(S)—N^{x}, —SO—, —SO₂—, n is 0 to 6 and W is C₁₋₆ hydrocarbyl, carbocycle or an aromatic heterocycle.

In another embodiment, where R¹ is a phenyl group it is preferred that it is not substituted by
(i) an azabicycle group as shown below: 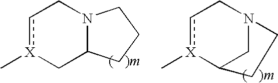
where [symbol] is a single bond when X is N, CH and is a double bond when X is C, and m is 1, 2, or 3, or
(ii) an azacycle as shown below: 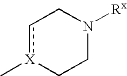
where [symbol] is a single bond when X is N, or is a single or double bond when X is C, or
(iii) [O, S, S(O), S(O)₂, NR^{x}, or CR^{x}═CR^{x}]—C(R^{x}R^{x})ₙ—R^{y} where R^{y} is a 5 to 7 ring membered heterocycle or NR^{z}R^{z} where R^{z} is hydrogen, C₁₋₆ alkyl, aralkyl and R^{x} is as defined above.

In another embodiment, where R¹ is an optionally substituted 5 or 6 membered aromatic carbocycle or heterocycle or a 9 to 10 ring membered bicyclic carbocyle or heterocycle it is preferred that one of its substituents is not:
(i) —O—(CH₂)₁₋₄CH₂NR¹⁰R¹⁰
(ii) —CH₂(CH₂)₀₋₄CH₂NR¹⁰R¹⁰
(iii) —CH(R¹⁰)(CH₂)₀₋₄CH₂NR¹⁰R¹⁰
(iv) —CH₂CH(CH₂NR¹⁰R¹⁰)(O-Ph)
(v) —O(CH₂)₁₋₂CH₂OR^{t}
(vi) —CH₂(CH₂)₀₋₂CH₂OR^{t}
(vii) —CH(R¹⁰)(CH₂)₀₋₂CH₂OR^{t}
(viii) —O(CH₂)₁₋₃CO₂R^{p}
(ix) —(CH₂)₀₋₃CO₂R^{p}
(x) —CH(R¹⁰)(CH₂)₀₋₃CO₂R^{p}
(xi) —OCH(R¹⁰)(CH₂)₀₋₃CO₂R^{p}
   where R^{t} is C(O)—C₁₋₈ alkyl, C(O)—C₃₋₈cycloalkyl, C(O)C₀₋₃ alkyl-carbocycyle or heterocycle and R^{p} is hydrogen, C₁₋₈alkyl, carbocycle or heterocycle, carbocycle-C₁₋₆alkyl, heterocycle-C₁₋₆alkyl, C₁₋₆alkylC(O)O C₁₋₆alkyl, carbocycleC(O)O C₁₋₆alkyl, heterocycleC(O)OC₁₋₆alkyl, carbocycle-C₁₋₆alkylC(O)O C₁₋₆alkyl, heterocycle-C₁₋₆alkylC(O)OC₁₋₆alkyl, C₁₋₈alkylNHC(O)C₁₋₆alkyl or C₁₋₈-dialkylNHC(O)C₁₋₆alkyl.

For the avoidance of doubt, it is to be understood that each general and specific preference, embodiment and example of the groups R¹ may be combined with each general and specific preference, embodiment and example of the groups X and/or A and/or R⁰ and/or R² and/or R³ and/or R⁴ and/or R⁵ and/or R⁶ and that all such combinations are embraced by this application.

For example, any one of the groups R¹ (e.g. as in R¹-A where A is C═O or NH(C═O)) shown in Table 1 may be combined with any one of the 4-oxo-4,5-dihydro-3H-imidazo[4,5-c]pyridine and 4-oxo-4,5-dihydro-1H-imidazo[4,5-d]pyridazin-2-yl groups shown in Table 2.

As in the preceding sections of this application, all references to formulae (Ia) and (Ib) should be taken to refer also to formula (II) and formula (III) and sub-formula thereof unless the context indicates otherwise.

Compounds of the Formula (I) Wherein R^{q} is a Group (c) and Compounds of the Formula (Ic)

The following preferences, embodiments and examples apply to X″, R¹ to R^{6b} and R¹⁰ in formula (I) where R^{q} is a group (c), and in formula (Ic).

In formula (I) where R^{q} is a group (c), and in formula (Ic), X″ can be NR^{4′}, O, S or S(O). In one particular embodiment, X″ is NR^{4′} or O. In another particular embodiment, X″ is O. Preferably X″ is NR^{4′}, more preferably NH. In another particular embodiment, X″ can be S or S(O), and more preferably S.

R¹ is hydrogen, a carbocyclic or heterocyclic group having from 3 to 12 ring members, or an optionally substituted C₁₋₈ hydrocarbyl group as hereinbefore defined. Examples of carbocyclic or heterocyclic groups and optionally substituted hydrocarbyl groups and general preferences for such groups are as set out above.

In one embodiment, R¹ is hydrogen, a carbocyclic or heterocyclic group having from 3 to 12 ring members, or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂.

In one embodiment, R¹ is an aryl or heteroaryl group.

When R¹ is a heteroaryl group, particular heteroaryl groups include monocyclic heteroaryl groups containing up to three heteroatom ring members selected from O, S and N, and bicyclic heteroaryl groups containing up to 2 heteroatom ring members selected from O, S and N and wherein both rings are aromatic. The heteroaryl groups may be unsubstituted or substituted by one or more substituent groups as hereinbefore defined.

Particular examples of R¹ include heteroaryl groups selected from pyrazolopyridinyl (e.g. pyrazolo[1,5-a]pyridin-3-yl), cinnoline, benzoisoxazole, furanyl (e.g. 2-furanyl and 3-furanyl), indolyl (e.g. 3-indolyl, 4-indolyl and 7-indolyl), oxazolyl, thiazolyl (e.g. thiazol-2-yl and thiazol-5-yl), isoxazolyl (e.g. isoxazol-3-yl and isoxazol-4-yl), pyrrolyl (e.g. 3-pyrrolyl), pyridyl (e.g. 2-pyridyl), quinolinyl (e.g. quinolin-8-yl), 2,3-dihydro-benzo[1,4]dioxine (e.g. 2,3-dihydro-benzo[1,4]dioxin-5-yl), benzo[1,3]dioxole (e.g. benzo[1,3]dioxol-4-yl), 2,3-dihydrobenzofuranyl (e.g. 2,3-dihydrobenzofuran-7-yl), imidazolyl and thiophenyl (e.g. 3-thiophenyl).

In one embodiment, R¹ is a bicyclic hetereroaryl group whereby the bicyclic group may contain two aromatic rings or an aromatic ring and a non-aromatic ring. Presently preferred R¹ heteroaryl groups include cinnoline, benzoisoxazole, 2,3-dihydrobenzofuranyl (e.g. 2,3-dihydrobenzofuran-7-yl), and pyrazolopyridine (e.g. pyrazolo[1,5-a]pyridine).

In one sub-group of compounds, R¹ is a bicyclic hetereroaryl group containing 2 heteroatoms independently selected from O and N, and wherein both rings are aromatic. Typically, at least one of the heteroatoms will be N. Preferred groups are a pyrazolo[1,5-a]pyridine group, such as a 3-pyrazolo[1,5-a]pyridinyl group, a cinnoline group such as cinnolin-4-yl and benzoisoxazole group such as benzo[c]isoxazol-3-yl.

In another subgroup of compounds, R¹ is a bicyclic hetereroaryl group whereby there is a phenyl ring with a non-aromatic heterocyclic group is fused to it. Preferred fused rings include oxa-, dioxa-, aza-, diaza- or oxa-aza-cycloalkyl groups. Preferably they form a cyclic group selected from those below.

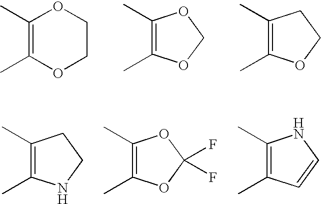

Typically the fused cycloalkyl group will contain an oxygen atom. Preferably the fused ring will be an oxa- or dioxa-cycloalkyl group such as one of those outlined below.

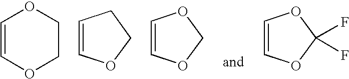

A particular example is 2,3-dihydrobenzofuranyl (e.g. 2,3-dihydrobenzofuran-7-yl).

In another embodiment, the group R¹ is a five membered heteroaryl group containing 1 or 2 ring heteroatoms selected from O, N and S. Particular heteroaryl groups include furan, thiophene, pyrrole, oxazole, isoxazole and thiazole groups. The heteroaryl groups may be unsubstituted or substituted by one or more substituent groups as hereinbefore defined.

A preferred R¹ aryl group is a phenyl ring.

Preferred non-aromatic groups R¹ include monocyclic cycloalkyl and azacycloalkyl groups such as cyclohexyl, cyclopentyl and piperidinyl, particularly cyclohexyl and 4-piperidinyl groups.

Particular examples of non-aromatic R¹ groups include unsubstituted or substituted (by one or more groups R¹⁰) monocyclic cycloalkyl groups such as cyclopropyl, cyclobutyl, cyclopentyl, cyclohexyl and cycloheptyl, particularly cyclohexyl, and unsubstituted or substituted (by one or more groups R¹⁰) 5-, 6- and 7-membered monocyclic heterocyclic groups such as morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, piperazine, and N-alkyl piperazines such as N-methyl piperazine. In general, preferred non-aromatic heterocyclic groups include pyrrolidine, piperidine, morpholine, thiomorpholine and N-methyl piperazine.

When R¹ is a C₁₋₈ hydrocarbyl group substituted by a carbocyclic or heterocyclic group, the carbocyclic and heterocyclic groups can be aromatic or non-aromatic and can be selected from the examples of such groups set out hereinabove.

When the carbocyclic or heterocylic group attached to the a C₁₋₈ hydrocarbyl group is aromatic, examples of such groups include monocyclic aryl groups and monocyclic heteroaryl groups containing up to four heteroatom ring members selected from O, S and N, and bicyclic heteroaryl groups containing up to 2 heteroatom ring members selected from O, S and N and wherein both rings are aromatic. Examples of such groups include furanyl (e.g. 2-furanyl or 3-furanyl), indolyl, oxazolyl, isoxazolyl, pyridyl, quinolinyl, pyrrolyl, imidazolyl and thienyl. Particular examples of aryl and heteroaryl groups as substituents for a C₁₋₈ hydrocarbyl group include phenyl, imidazolyl, tetrazolyl, triazolyl, indolyl, 2-furanyl, 3-furanyl, pyrrolyl and thienyl.

When R¹ is a C₁₋₈ hydrocarbyl group substituted by a non-aromatic carbocyclic or heterocyclic group, the non-aromatic or heterocyclic group may be a group selected from the lists of such groups set out hereinabove. For example, the non-aromatic group can be a monocyclic group having from 5 to 7 ring members and typically containing from 0 to 3, more typically 0, 1 or 2, heteroatom ring members selected from O, S and N. Particular examples include monocyclic cycloalkyl groups such as cyclopropyl, cyclobutyl, cyclopentyl, cyclohexyl and cycloheptyl, and 5-, 6- and 7-membered monocyclic heterocyclic groups such as morpholine, piperidine (e.g. 1-piperidinyl, 2-piperidinyl 3-piperidinyl and 4-piperidinyl), pyrrolidine (e.g. 1-pyrrolidinyl, 2-pyrrolidinyl and 3-pyrrolidinyl), pyrrolidone, piperazine, and N-alkyl piperazines such as N-methyl piperazine. In general, preferred non-aromatic heterocyclic groups include pyrrolidine, piperidine, morpholine, thiomorpholine and N-methyl piperazine.

In one embodiment R¹ is an unsubstituted carbocyclic or heterocyclic group.

When R¹ is an optionally substituted C₁₋₈ hydrocarbyl group, the hydrocarbyl group may be as hereinbefore defined, and is preferably up to four carbon atoms in length, more usually up to three carbon atoms in length for example one or two carbon atoms in length. In one embodiment, the hydrocarbyl group is a linear saturated group having from 1-6 carbon atoms, more usually 1-4 carbon atoms, for example 1-3 carbon atoms, e.g. 1, 2 or 3 carbon atoms. When the hydrocarbyl group is substituted, particular examples of such groups are substituted (e.g. by a carbocyclic such as phenyl or a heterocyclic group) methyl and ethyl groups. A preferred substituted C₁₋₈ hydrocarbyl R¹ group is aralkyl groups such as phenyl.

Preferred substituted and unsubstituted C₁₋₈ hydrocarbyl groups include trifluoromethyl and tertiary butyl groups.

Particularly preferred R¹ groups are phenyl groups.

The group R¹ can be an unsubstituted or substituted carbocyclic or heterocyclic group in which one or more substituents can be selected from the group R¹⁰ as hereinbefore defined. In one embodiment, the substituents on R¹ may be selected from the group R^{10a} consisting of halogen, hydroxy, trifluoromethyl, cyano, nitro, carboxy, heterocyclic groups having 5 or 6 ring members and up to 2 heteroatoms selected from O, N and S, a group R^{a}-R^{b} wherein R^{a} is a bond, O, CO, X³C(X⁴), C(X⁴)X³, X³C(X⁴)X³, S, SO, or SO₂, and R^{b} is selected from hydrogen, heterocyclic groups having 5 or 6 ring members and up to 2 heteroatoms selected from O, N and S, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having 5 or 6 ring members and up to 2 heteroatoms selected from O, N and S; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In a further embodiment, the substituents on R¹ may be selected from the group R^{10b} consisting of halogen, hydroxy, trifluoromethyl, cyano, nitro, carboxy, a group R^{a}-R^{b} wherein R^{a} is a bond, O, CO, X³C(X⁴), C(X⁴)X³, X³C(X⁴)X³, S, SO, or SO₂, and R^{b} is selected from hydrogen and a C₁₋₈ hydrocarbyl group optionally substituted by one or more substituents selected from hydroxy, oxo, halogen, cyano, nitro, carboxy; wherein one or more carbon atoms of the C₁₋₈ hydrocarbyl group may optionally be replaced by O, S, SO, SO₂, X³C(X⁴), C(X⁴)X³ or X³C(X⁴)X³; X³ is O or S; and X⁴ is ═O or ═S.

In another embodiment, the substituents on R¹ may be selected from halogen, hydroxy, trifluoromethyl, a group R^{a}-R^{b} wherein R^{a} is a bond or O, and R^{b} is selected from hydrogen and a C₁₋₄ hydrocarbyl group optionally substituted by one or more substituents selected from hydroxyl and halogen (preferably fluorine).

Particular examples of substituents that may be present on a group R¹ (e.g. an aryl or heteroaryl group R¹) include fluorine, chlorine, methoxy, methyl, oxazolyl, morpholino, trifluoromethyl, bromomethyl, chloroethyl, pyrrolidino, pyrrolidinylethoxy, pyrrolidinylmethyl, difluoromethoxy and morpholinomethyl.

The moiety R¹ may be substituted by more than one substituent. Thus, for example, there may be 1 or 2 or 3 or 4 substituents, more typically 1, 2 or 3 substituents. In one embodiment, where R¹ is a six membered ring (e.g. a carbocyclic ring such as a phenyl ring), there may be a single substituent which may be located at any one of the 2-, 3- and 4-positions on the ring. In another embodiment, there may be two or three substituents and these may be located at the 2-, 3-, 4-, 5- or 6-positions around the ring.

By way of example, a phenyl group R¹ may be 2-monosubstituted, 3-monosubstituted, 2,6-disubstituted, 2,3-disubstituted, 2,4-disubstituted, 2,5-disubstituted, 2,3,6-trisubstituted or 2,4,6-trisubstituted. More particularly, a phenyl group R¹ may be be monosubstituted at the 2-position or disubstituted at positions 2- and 6- with substituents selected from fluorine, chlorine and R^{a}-R^{b}, where R^{a} is O and R^{b} is C₁₋₄ alkyl (e.g. methyl or ethyl), with fluorine, chlorine and methoxy being particularly preferred substituents.

In another group of preferred compounds, the phenyl group R¹ is 2,4-disubstituted or 2,5-disubstituted. The 2-substituent may be, for example, a halogen (e.g. F or Cl) or a methoxy group. In one particular group of compounds, the 2-substituent is methoxy. The 5-substituent, when present, can be selected from, for example, halogen (e.g. Cl or F), C₁₋₄ alkyl (e.g. tert-butyl or isopropyl), methoxy, trifluoromethoxy, trifluoromethyl, or a group HetN-SO₂— where “HetN” is a nitrogen-containing saturated monocyclic heterocycle such as piperazino, N—C₁₋₄ alkylpiperazino, morpholino, piperidino or pyrrolidino. One preferred 5-substitutent is Cl, and a preferred 2,5-combination is 2-methoxy-5-chlorophenyl.

In a further group of compounds, the phenyl group R¹ has a single substituent at the 4-position of the phenyl ring. The substituent can be, for example, a halogen atom (preferably fluorine or chlorine, most preferably fluorine) or a trifluoromethyl group.

Particular examples of groups R¹ include the groups A1 to A62 set out in Table 1 above.

Preferred groups R¹ include groups A1 to A10, A18, A61 and A62 in Table 1. Typically R¹ is selected from A1, A3, A4, A5, A8, A10, A18, A61 and A62.

Particularly preferred groups R¹ include 2,6-difluorophenyl, 2-fluoro-6-methoxyphenyl, 2,6-dichlorophenyl, 2,4,6-trifluorophenyl, 2,3-dihydro-benzo[1,4]furan-7-yl, cinnolin-4-yl, benzo[c]isoxazol-3-yl and pyrazolo[1,5-a]pyridin-3-yl.

A currently most preferred group R¹ is 2,6-difluorophenyl.

R² is hydrogen, halogen, methoxy, or a C₁₋₄ hydrocarbyl group optionally substituted by halogen, hydroxyl or methoxy. Preferably R² is hydrogen, chlorine or methyl, and most preferably R² is hydrogen.

The moieties R^{3a}, R^{3b}, R^{5a}, and R^{5b} are typically selected from hydrogen, trifluoromethyl, cyano, carboxy, monocyclic carbocyclic and heterocyclic groups having from 3 to 12 (preferably 3 to 7, and more typically 5 or 6) ring members, a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, a carbocyclic or heterocyclic group with 3-7 ring members and a C₁₋₈ hydrocarbyl group optionally substituted by one or more substituents selected from hydroxy, C₁₋₄ acyloxy, oxo, halogen, cyano, nitro, carboxy, amino, mono- or di-C₁₋₄ hydrocarbylamino, a carbocyclic or heterocyclic group with 3-7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; where R^{c}, X¹ and X² are as before defined.

In one embodiment, R^{3a}, R^{3b}, R^{5a}, and R^{5b} are each hydrogen or are selected from cyano, trifluoromethyl, a group R^{a}-R^{b} wherein R^{a} is a bond, O, CO or C(X²)X¹ and R^{b} is selected from hydrogen, heterocyclic groups having from 3 to 12 ring members (preferably 4 to 7 ring members), and a C₁₋₈ hydrocarbyl group (preferably a C₁₋₄ hydrocarbyl group), optionally substituted by one or more substituents selected from hydroxy, C₁₋₄ acyloxy, mono- or di-C₁₋₄ hydrocarbylamino, heterocyclic groups having from 3 to 12 ring members, more preferably 4 to 7 ring members; where R^{c} is selected from hydrogen and C₁₋₄ hydrocarbyl, X¹ is O or NR^{c} and X² is ═O.

In another embodiment, R^{3a}, R^{3b}, R^{5a}, and R^{5b} are selected from hydrogen, trifluoromethyl, carboxy, a group R^{a}-R^{b} wherein R^{a} is a bond, O, CO, C(X²)X¹, and R^{b} is selected from hydrogen, heterocyclic groups having 3-7 ring members (e.g. pyrrolidine, N-methyl piperazine or morpholine) and a C₁₋₄ hydrocarbyl group optionally substituted by one or more substituents selected from hydroxy, carboxy, C₁₋₄ acyloxy, amino, mono- or di-C₁₋₄ hydrocarbylamino, heterocyclic groups with 3-7 ring members (e.g. pyrrolidine, N-methyl piperazine or morpholine).

In a more preferred embodiment, R^{3a}, R^{3b}, R^{5a}, and R^{5b} are selected from hydrogen, trifluoromethyl, a group R^{a}-R^{b} wherein R^{a} is a bond, O, CO, C(X²)X¹, and R^{b} is selected from hydrogen, saturated heterocyclic groups having 5-6 ring members and a C₁₋₂ hydrocarbyl group optionally substituted by one or more substituents selected from hydroxy, carboxy, C₁₋₂ acyloxy, amino, mono- or di-C₁₋₄ hydrocarbylamino, heterocyclic groups with 5-6 ring members.

In another embodiment, particular substituent groups R^{3a}, R^{3b}, R^{5a}, and R^{5b} include hydrogen and a group R^{a}-R^{b} wherein R^{a} is a bond, O, CO, C(X²)X¹, and R^{b} is selected from hydrogen, heterocyclic group having 3-7 ring members and a C₁₋₄ hydrocarbyl group optionally substituted by one or more substituents selected from hydroxy, carboxy, amino, mono- or di-C₁₋₄ hydrocarbylamino, heterocyclic group with 3-7 ring members.

Typically R^{3a}, R^{3b}, R^{5a}, and R^{5b} are all hydrogen.

The group R^{4′} is selected from hydrogen, trifluoromethyl, carbocyclic and heterocyclic groups having from 3 to 12 ring members; a group R^{d′}-R^{e′} wherein R^{d′} is a bond, CO, C(X²)X¹, SO, SO₂, or SO₂NR^{c}; and R^{e′} is selected from, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 7 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

Preferably R^{4′} is selected from hydrogen and a group R^{d′}-R^{e′} wherein R^{d′} is a bond, CO, C(X²)X¹, or SO₂; and R^{e′} is selected from carbocyclic and heterocyclic groups having from 3 to 7 ring members, and a C₁₋₄ hydrocarbyl group optionally substituted by one or more substituents selected from hydroxy, alkoxy, halogen, amino, mono- or di-C₁₋₄ hydrocarbylamino, carbocyclic and heterocyclic groups having from 3 to 7 ring members.

In one embodiment R^{4′} is selected from hydrogen and a group R^{d′}-R^{e′} wherein R^{d′} is a bond and R^{e′} is a C₁₋₄ hydrocarbyl group optionally substituted by one or more substituents selected from hydroxy, alkoxy, halogen preferably fluorine, carbocyclic and heterocyclic groups having from 3 to 7 ring members.

In another embodiment, R^{4′} is selected from hydrogen and unsubstituted or substituted C₁₋₄ alkyl group where the substituents are alkoxy such as methoxy or halogen such as fluorine.

Preferably R^{4′} is selected from hydrogen, unsubstituted methyl, unsubstituted ethyl, unsubstituted 2-methoxy-ethyl, unsubstituted 2-fluoro-ethyl, and unsubstituted 2,2-difluoro-ethyl. Typically R^{4′} is hydrogen.

In one sub-group of compounds, R^{4′} is selected from hydrogen, unsubstituted methyl, unsubstituted 2-methoxy-ethyl, unsubstituted 2-fluoro-ethyl, and unsubstituted 2,2-difluoro-ethyl. Typically R^{4′} is hydrogen.

The moieties R^{6a} and R^{6b} are typically selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

In one embodiment, R^{6a} and R^{6b} are each hydrogen or are selected from halogen, cyano, hydroxy, trifluoromethyl, a group R^{a}-R^{b} wherein R^{a} is a bond, O, CO or C(X²)X¹ and R^{b} is selected from hydrogen, heterocyclic groups having from 3 to 12 ring members (preferably 4 to 7 ring members), and a C₁₋₈ hydrocarbyl group (preferably a C₁₋₄ hydrocarbyl group), optionally substituted by one or more substituents selected from hydroxy, C₁₋₄ acyloxy, mono- or di-C₁₋₄ hydrocarbylamino, heterocyclic groups having from 3 to 12 ring members, more preferably 4 to 7 ring members; where R^{c} is selected from hydrogen and C₁₋₄ hydrocarbyl, X¹ is O or NR^{c} and X² is ═O.

In another embodiment, R^{6a} and R^{6b} are selected from hydrogen, fluorine, chlorine, bromine, trifluoromethyl, carboxy, a group R^{a}-R^{b} wherein R^{a} is a bond, O, CO, C(X²)X¹, and R^{b} is selected from hydrogen, heterocyclic groups having 3-7 ring members (e.g. pyrrolidine, N-methyl piperazine or morpholine) and a C₁₋₄ hydrocarbyl group optionally substituted by one or more substituents selected from hydroxy, carboxy, C₁₋₄ acyloxy, amino, mono- or di-C₁₋₄ hydrocarbylamino, heterocyclic groups with 3-7 ring members (e.g. pyrrolidine, N-methyl piperazine or morpholine).

In a more preferred embodiment, R^{6a} and R^{6b} are selected from hydrogen, fluorine, chlorine, trifluoromethyl, a group R^{a}-R^{b} wherein R^{a} is a bond, O, CO, C(X²)X¹, and R^{b} is selected from hydrogen, saturated heterocyclic groups having 5-6 ring members and a C₁₋₂ hydrocarbyl group optionally substituted by one or more substituents selected from hydroxy, carboxy, C₁₋₂ acyloxy, amino, mono- or di-C₁₋₄ hydrocarbylamino, heterocyclic groups with 5-6 ring members.

In another embodiment, particular substituent groups R^{6a} and R^{6b} include hydrogen, halogen, a group R^{a}-R^{b} wherein R^{a} is a bond, O, CO, C(X²)X¹, and R^{b} is selected from hydrogen, heterocyclic group having 3-7 ring members and a C₁₋₄ hydrocarbyl group optionally substituted by one or more substituents selected from hydroxy, carboxy, amino, mono- or di-C₁₋₄ hydrocarbylamino, heterocyclic group with 3-7 ring members.

Typically R^{6a} and R^{6b} are hydrogen.

Whereas each of R^{3a}, R^{3b}, R^{5a}, R^{5b}, R^{6a} or R^{6b} can be hydrogen or a substituent as hereinbefore defined other than hydrogen, it is preferred that at least one, more preferably at least two, three, four or five of R^{3a}, R^{3b}, R^{5a}, R^{5b}, R^{6a} or R^{6b} are hydrogen.

In one particular embodiment, one of R^{3a}, R^{3b}, R^{5a}, R^{5b}, R^{6a} or R^{6b} is a substituent other than hydrogen and the others each are hydrogen. For example, R^{3a} can be a other than hydrogen and R^{3b}, R^{5a}, R^{5b}, R^{6a} and R^{6b} can each be hydrogen, or R^{5a} can be other than hydrogen and R^{3a}, R^{3b}, R^{5b}, R^{6a} and R^{6b} can each be hydrogen.

In another particular embodiment, two of R^{3a}, R^{3b}, R^{5a}, R^{5b}, R^{6a} or R^{6b} are other than hydrogen and the others are hydrogen. For example, R^{3a} and R^{5a} can both be other than hydrogen when R^{3b}, R^{5b}, R^{6a} and R^{6b} are hydrogen; or R^{3b} and R^{5a} can both be other than hydrogen when R^{3b}, R^{5b}, R^{6a} and R^{6b} are hydrogen; or R^{3a} and R^{6b} can both be other than hydrogen when R^{3b}, R^{5a}, R^{5b}, and R^{6a} are hydrogen, and the like.

In a further embodiment, it is preferred that R^{4′} is a substituent as defined herein other than hydrogen and that at least one, more preferably at least two, three, four or five of R^{3a}, R^{3b}, R^{5a}, R^{5b}, R^{6a} or R^{6b} are hydrogen.

In one particular embodiment, R^{4′} is other than hydrogen and one of R^{3a}, R^{3b}, R^{5a}, R^{5b}, R^{6a} or R^{6b} is other than hydrogen and the others each are hydrogen. For example, R^{3a} and R^{4′} are other than hydrogen and R^{3b}, R^{5a}, R^{5b}, R^{6a} and R^{6b} can each be hydrogen, or R^{5a} and R^{4′} can be other than hydrogen and R^{3a}, R^{3b}, R^{5b}, R^{6a} and R^{6b} can each be hydrogen.

In another particular embodiment, R^{4′} is other than hydrogen and two of R^{3a}, R^{3b}, R^{5a}, R^{5b}, R^{6a} or R^{6b} are other than hydrogen and the others are hydrogen. For example, R^{3a}, R^{4′} and R^{5a} can be other than hydrogen when R^{3b}, R^{5b}, R^{6a} and R^{6b} are hydrogen; or R^{3b}, R^{4′} and R^{5a} can be other than hydrogen when R^{3b}, R^{5b}, R^{6a} and R^{6b} are hydrogen; or R^{3a}, R^{4′} and R^{6b} can be other than hydrogen when R^{3b}, R^{5a}, R^{5b}, and R^{6a} are hydrogen, and the like.

In one particularly preferred embodiment one of R^{3a} or R^{3b} is hydrogen and the other is other than hydrogen, one of R^{5a} or R^{5b} is hydrogen and the other is other than hydrogen, and one of R^{6a} or R^{6b} is hydrogen and the other is other than hydrogen.

In another particularly preferred embodiment both of R^{3a} and R^{3b} are other than hydrogen, one of R^{5a} or R^{5b} is hydrogen and the other is other than hydrogen and both of R^{6a} and R^{6b} are hydrogen.

R^{3a} and R^{3b} are preferably independently selected from:

hydrogen;

methyl optionally substituted by a substituent selected from hydroxy, halogen (e.g. fluorine, preferably difluoro or trifluoro, and more preferably trifluoro) and

NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R^{4′} is preferably selected from:

hydrogen; and

alkyl such as methyl or ethyl, optionally substituted by a substituent selected from hydroxy, alkoxy (e.g. methoxy), and halogen (e.g. fluorine, preferably difluoro or trifluoro, and more preferably mono or difluoro).

R^{5a} and R^{5b} are preferably independently selected from:

hydrogen;

C₁₋₄ alkoxy (for example methoxy);

methyl optionally substituted by a substituent selected from hydroxy, halogen (e.g. fluorine, preferably difluoro or trifluoro, and more preferably trifluoro) and NR¹¹R¹²; and

C(═O)NR¹¹R¹²;

wherein R¹¹ and R¹² are the same or different and each is selected from hydrogen and C₁₋₄ alkyl or R¹¹ and R¹² together with the nitrogen atom form a five or six membered heterocyclic ring having 1 or 2 heteroatom ring members selected from O, N and S (preferably O and N).

R^{6a} and R^{6b} are preferably independently selected from hydrogen, fluorine and methyl, most preferably hydrogen.

In the foregoing definitions, when R¹¹ and R¹² together with the nitrogen atom in the group NR¹¹R¹² form a five or six membered heterocyclic ring, the heteroatom ring members are preferably selected from O and N. The heterocyclic ring is typically non-aromatic and examples of such rings include morpholine, piperazine, N—C₁₋₄-alkylpiperazine, piperidine and pyrrolidine. Particular examples of N—C₁₋₄-alkylpiperazine groups include N-methylpiperazine and N-isopropylpiperazine.

Preferred X″, R^{3a}, R^{3b}, R^{4′}, R^{5a}, R^{5b}, R^{6a} and R^{6b} groups include those in which the 4,5,6,7-tetrahydroimidazo[4,5-c]pyridine group below:

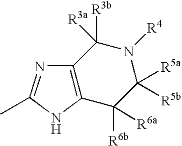

is as shown in Table 3 below.

TABLE 3

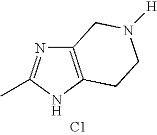

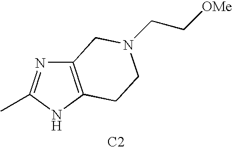

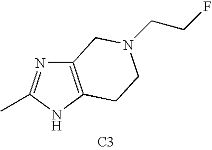

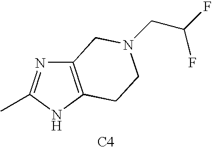

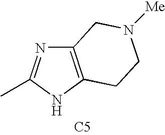

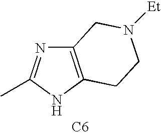

Of the 4,5,6,7-tetrahydroimidazo[4,5-c]pyridine groups set out in Table 3 above, particular groups include the group C1. Other particulars group are groups C3 and C6.

Further preferred compounds of the formula (I) include those in which the moiety (c) is C7 as shown below.

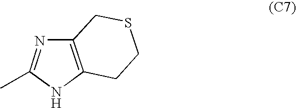

One preferred group of compounds of the invention is represented by the formula (IV): 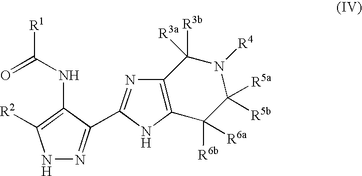
wherein R¹, R², R^{3a}, R^{3b}, R^{5a}, R^{5b}, R^{6a} and R^{6b} are independently selected from R¹, R², R^{3a}, R^{3b}, R^{5a}, R^{5b}, R^{6a} and R^{6b} or sub-groups thereof as herein defined.

Within formula (IV), it is preferred that R² is hydrogen or C₁₋₄ alkyl, and more typically R² is hydrogen.

Within the group of compounds defined by the formula (IV), R¹ is preferably 2-substituted, 2,6 disubstituted or 2,4,6, trisubstituted phenyl or a bicyclic heteraryl group, where the substituents are selected from halogen and C₁₋₄ alkoxy.

More preferably R¹ is selected from 2-methoxyphenyl, 2,6-difluorophenyl, 2-fluoro-6-methoxyphenyl, 2,6-dichlorophenyl, 2,4,6-trifluorophenyl, cinnoline (e.g. cinnolin-4-yl), benzoisoxazole (e.g. such as benzo[c]isoxazol-3-yl.), 2,3-dihydrobenzofuranyl (e.g. 2,3-dihydrobenzofuran-7-yl), and pyrazolopyridine (e.g. pyrazolo[1,5-a]pyridine group, such as a 3-pyrazolo[1,5-a]pyridinyl group).

One particularly preferred group R¹ is 2,6-difluorophenyl.

Within Formula (IV), it is preferred that R^{3a}, R^{3b}, R^{5a}, and R^{5b} are independently selected from hydrogen, and a group R^{a}-R^{b} wherein R^{a} is a bond, O, CO, C(X²)X¹, and R^{b} is selected from hydrogen, heterocyclic group having 3-7 ring members and a C₁₋₄ hydrocarbyl group optionally substituted by one or more substituents selected from hydroxy, carboxy, amino, mono- or di-C₁₋₄ hydrocarbylamino, heterocyclic group with 3-7 ring members.

Within Formula (IV), it is preferred that R^{6a} and R^{6b} are independently selected from hydrogen, halogen, a group R^{a}-R^{b} wherein R^{a} is a bond, O, CO, C(X²)X¹, and R^{b} is selected from hydrogen, heterocyclic group having 3-7 ring members and a C₁₋₄ hydrocarbyl group optionally substituted by one or more substituents selected from hydroxy, carboxy, amino, mono- or di-C₁₋₄ hydrocarbylamino, heterocyclic group with 3-7 ring members.

More preferably R^{3a}, R^{3b}, R^{5a}, R^{5b}, R^{6a} and R^{6b} are hydrogen.

It is preferred that R^{4′} groups is selected from hydrogen or unsubstituted or substituted C₁₋₄ alkyl group where the substituents are alkoxy such as methoxy or halogen such as fluorine.

Preferably R^{4′} is selected from hydrogen, unsubstituted methyl, unsubstituted 2-methoxy-ethyl, unsubstituted 2-fluoro-ethyl, and unsubstituted 2,2-difluoro-ethyl. Typically R⁴ is hydrogen.

Compounds of formula (I) wherein X″ is S are particularly preferred as aurora kinase inhibitors.

For the avoidance of doubt, it is to be understood that each general and specific preference, embodiment and example of the groups R¹ may be combined with each general and specific preference, embodiment and example of the groups R² and/or R^{3a} and/or R^{3b}, and/or R^{4′} and/or R^{5a}, and/or R^{5b}, and/or R^{6a} and/or R^{6b} and/or R¹⁰ and/or R⁰ and/or X″ and/or A and that all such combinations are embraced by this application.

For example, any one of the groups R¹ (e.g. as in R¹-A where A is C═O) shown in Table 1 may be combined with any one of the 4,5,6,7-tetrahydroimidazo[4,5-c]-pyridine groups shown in Table 3.

The various functional groups and substituents making up the compounds of the formulae (I), (Ia), (Ib), (Ic), (II), (III) and (IV) are typically chosen such that the molecular weight of the compound of the formula (I) does not exceed 1000. More usually, the molecular weight of the compound will be less than 750, for example less than 700, or less than 650, or less than 600, or less than 550. More preferably, the molecular weight is less than 525 and, for example, is 500 or less.

Particular compounds of the invention are:
2,6-difluoro-N-[3-(4-oxo-4,5-dihydro-3H-imidazo[4,5-c]pyridin-2-yl)-1H-pyrazol-4-yl]-benzamide;
5-methyl-4-morpholin-4-ylmethyl-furan-2-carboxylic acid [3-(5-benzyl-4-oxo-4,5 dihydro-1H-imidazo[4,5-d]pyridazin-2-yl)-1H-pyrazol-4-yl]-amide;
5-methyl-4-morpholin-4-ylmethyl-furan-2-carboxylic acid [3-(5-methyl-4-oxo-4,5-dihydro-1H-imidazo[4,5-d]pyridazin-2-yl)-1H-pyrazol-4-yl]-amide;
2,6-difluoro-N-[3-(4,5,6,7-tetrahydro-1H-imidazo[4,5-c]pyridin-2-yl)-1H-pyrazol-4-yl]-benzamide;
2,6-dichloro-N-[3-(4,5,6,7-tetrahydro-1H-imidazo[4,5-c]pyridin-2-yl)-1H-pyrazol-4-yl]-benzamide;
2,4,6-trifluoro-N-[3-(4,5,6,7-tetrahydro-1H-imidazo[4,5-c]pyridin-2-yl)-1H-pyrazol-4-yl]-benzamide;
2-methoxy-N-[3-(4,5,6,7-tetrahydro-1H-imidazo[4,5-c]pyridin-2-yl)-1H-pyrazol-4-yl]-benzamide;
2-fluoro-6-methoxy-N-[3-(4,5,6,7-tetrahydro-1H-imidazo[4,5-c]pyridin-2-yl)-1H-pyrazol-4-yl]-benzamide;
pyrazolo[1,5-a]pyridine-3-carboxylic acid [3-(4,5,6,7-tetrahydro-1H-imidazo[4,5-c]pyridin-2-yl)-1H-pyrazol-4-yl]-amide;
benzo[c]isoxazole-3-carboxylic acid [3-(4,5,6,7-tetrahydro-1H-imidazo[4,5-c]pyridin-2-yl)-1H-pyrazol-4-yl]-amide;
cinnoline-4-carboxylic acid [3-(4,5,6,7-tetrahydro-1H-imidazo[4,5-c]pyridin-2-yl)-1H-pyrazol-4-yl]-amide;
2,3-dihydro-benzofuran-7-carboxylic acid [3-(4,5,6,7-tetrahydro-1H-imidazo[4,5-c]pyridin-2-yl)-1H-pyrazol-4-yl]-amide;
2,6-difluoro-N-[3-(5-methyl-4,5,6,7-tetrahydro-1H-imidazo[4,5-c]pyridin-2-yl)-1H-pyrazol-4-yl]-benzamide;
2,6-dichloro-N-[3-(5-methyl-4,5,6,7-tetrahydro-1H-imidazo[4,5-c]pyridin-2-yl)-1H-pyrazol-4-yl]-benzamide;
2,4,6-trifluoro-N-[3-(5-methyl-4,5,6,7-tetrahydro-1H-imidazo[4,5-c]pyridin-2-yl)-1H-pyrazol-4-yl]-benzamide;
2-methoxy-N-[3-(5-methyl-4,5,6,7-tetrahydro-1H-imidazo[4,5-c]pyridin-2-yl)-1H-pyrazol-4-yl]-benzamide;
2-fluoro-6-methoxy-N-[3-(5-methyl-4,5,6,7-tetrahydro-1H-imidazo[4,5-c]pyridin-2-yl)-1H-pyrazol-4-yl]-benzamide;
pyrazolo[1,5-a]pyridine-3-carboxylic acid [3-(5-methyl-4,5,6,7-tetrahydro-1H-imidazo[4,5-c]pyridin-2-yl)-1H-pyrazol-4-yl]-amide;
benzo[c]isoxazole-3-carboxylic acid [3-(5-methyl-4,5,6,7-tetrahydro-1H-imidazo[4,5-c]pyridin-2-yl)-1H-pyrazol-4-yl]-amide;
cinnoline-4-carboxylic acid [3-(5-methyl-4,5,6,7-tetrahydro-1H-imidazo[4,5-c]pyridin-2-yl)-1H-pyrazol-4-yl]-amide;
2,3-dihydro-benzofuran-7-carboxylic acid [3-(5-methyl-4,5,6,7-tetrahydro-1H-imidazo[4,5-c]pyridin-2-yl)-1H-pyrazol-4-yl]-amide;
2,6-difluoro-N-[3-(5-{2-fluoro-ethyl}-4,5,6,7-tetrahydro-1H-imidazo[4,5-c]pyridin-2-yl)-1H-pyrazol-4-yl]-benzamide;
2,6-dichloro-N-[3-(5-{2-fluoro-ethyl}-4,5,6,7-tetrahydro-1H-imidazo[4,5-c]pyridin-2-yl)-1H-pyrazol-4-yl]-benzamide;
2,4,6-trifluoro-N-{3-[5-(2-fluoro-ethyl)-4,5,6,7-tetrahydro-1H-imidazo[4,5-c]pyridin-2-yl]-1H-pyrazol-4-yl}-benzamide;
2-methoxy-N-[3-(5-{2-fluoro-ethyl}-4,5,6,7-tetrahydro-1H-imidazo[4,5-c]pyridin-2-yl)-1H-pyrazol-4-yl]-benzamide;
2-fluoro-6-methoxy-N-[3-(5-{2-fluoro-ethyl}-4,5,6,7-tetrahydro-1H-imidazo[4,5-c]pyridin-2-yl)-1H-pyrazol-4-yl]-benzamide;
pyrazolo[1,5-a]pyridine-3-carboxylic acid [3-(5-{2-fluoro-ethyl}-4,5,6,7-tetrahydro-1H-imidazo[4,5-c]pyridin-2-yl)-1H-pyrazol-4-yl]-amide;
benzo[c]isoxazole-3-carboxylic acid [3-(5-{2-fluoro-ethyl}-4,5,6,7-tetrahydro-1H-imidazo[4,5-c]pyridin-2-yl)-1H-pyrazol-4-yl]-amide;
cinnoline-4-carboxylic acid [3-(5-{2-fluoro-ethyl}-4,5,6,7-tetrahydro-1H-imidazo[4,5-c]pyridin-2-yl)-1H-pyrazol-4-yl]-amide;
2,3-dihydro-benzofuran-7-carboxylic acid {3-[5-(2-fluoro-ethyl)-4,5,6,7-tetrahydro-1H-imidazo[4,5-c]pyridin-2-yl]-1H-pyrazol-4-yl}-amide;
N-{3-[5-(2,2-difluoro-ethyl)-4,5,6,7-tetrahydro-1H-imidazo[4,5-c]pyridin-2-yl]-1H-pyrazol-4-yl}-2,6-difluoro-benzamide;
2,6-dichloro-N-{3-[5-(2,2-difluoro-ethyl)-4,5,6,7-tetrahydro-1H-imidazo[4,5-c]pyridin-2-yl]-1H-pyrazol-4-yl}-benzamide;
N-{3-[5-(2,2-difluoro-ethyl)-4,5,6,7-tetrahydro-1H-imidazo[4,5-c]pyridin-2-yl]-1H-pyrazol-4-yl}-2,4,6-trifluoro-benzamide;
N-{3-[5-(2,2-difluoro-ethyl)-4,5,6,7-tetrahydro-1H-imidazo[4,5-c]pyridin-2-yl]-1H-pyrazol-4-yl}-2-methoxy-benzamide;
N-{3-[5-(2,2-difluoro-ethyl)-4,5,6,7-tetrahydro-1H-imidazo[4,5-c]pyridin-2-yl]-1H-pyrazol-4-yl}-2-fluoro-6-methoxy-benzamide;
pyrazolo[1,5-a]pyridine-3-carboxylic acid {3-[5-(2,2-difluoro-ethyl)-4,5,6,7-tetrahydro-1H-imidazo[4,5-c]pyridin-2-yl]-1H-pyrazol-4-yl}-amide;
benzo[c]isoxazole-3-carboxylic acid {3-[5-(2,2-difluoro-ethyl)-4,5,6,7-tetrahydro-1H-imidazo[4,5-c]pyridin-2-yl]-1H-pyrazol-4-yl}-amide;
cinnoline-4-carboxylic acid {3-[5-(2,2-difluoro-ethyl)-4,5,6,7-tetrahydro-1H-imidazo[4,5-c]pyridin-2-yl]-1H-pyrazol-4-yl}-amide;
2,3-dihydro-benzofuran-7-carboxylic acid {3-[5-(2,2-difluoro-ethyl)-4,5,6,7-tetrahydro-1H-imidazo[4,5-c]pyridin-2-yl]-1H-pyrazol-4-yl}-amide;
2,6-difluoro-N-{3-[5-(2-methoxy-ethyl)-4,5,6,7-tetrahydro-1H-imidazo[4,5-c]pyridin-2-yl]-1H-pyrazol-4-yl}-benzamide;
2,6-dichloro-N-{3-[5-(2-methoxy-ethyl)-4,5,6,7-tetrahydro-1H-imidazo[4,5-c]pyridin-2-yl]-1H-pyrazol-4-yl}-benzamide;
2,4,6-trifluoro-N-{3-[5-(2-methoxy-ethyl)-4,5,6,7-tetrahydro-1H-imidazo[4,5-c]pyridin-2-yl]-1H-pyrazol-4-yl}-benzamide;
2-methoxy-N-{3-[5-(2-methoxy-ethyl)-4,5,6,7-tetrahydro-1H-imidazo[4,5-c]pyridin-2-yl]-1H-pyrazol-4-yl}-benzamide;
2-fluoro-6-methoxy-N-{3-[5-(2-methoxy-ethyl)-4,5,6,7-tetrahydro-1H-imidazo[4,5-c]pyridin-2-yl]-1H-pyrazol-4-yl}-benzamide;
benzo[c]isoxazole-3-carboxylic acid {3-[5-(2-methoxy-ethyl)-4,5,6,7-tetrahydro-1H-imidazo[4,5-c]pyridin-2-yl]-1H-pyrazol-4-yl}-amide;
cinnoline-4-carboxylic acid {3-[5-(2-methoxy-ethyl)-4,5,6,7-tetrahydro-1H-imidazo[4,5-c]pyridin-2-yl]-1H-pyrazol-4-yl}-amide;
2,3-dihydro-benzofuran-7-carboxylic acid {3-[5-(2-methoxy-ethyl)-4,5,6,7-tetrahydro-1H-imidazo[4,5-c]pyridin-2-yl]-1H-pyrazol-4-yl}-amide;
2,6-difluoro-N-[3-(4,5,6,7-tetrahydro-1H-imidazol[4,5-c]pyridin-2-yl)-1H-pyrazol-4-yl]-benzamide;
2,6-difluoro-N-[3-(1,4,6,7-tetrahydro-thiopyrano[3,4-d]imidazol-2-yl)-1H-pyrazol-4-yl]-benzamide;
S-(−)-tetrahydro-furan-2-carboxylic acid {3-[5-(2-morpholin-4-yl-ethyl)-4,5-dihydro-1H-imidazo[4,5-d]pyridazin-2-yl]-1H-pyrazol-4-yl}-amide; and
N-[3-(5-ethyl-4,5,6,7-tetrahydro-1H-imidazo[4,5-c]pyridine-2-yl)-1H-pyrazol-4-yl]-2,6-difluoro-benzamide;
   salts, solvates, tautomers and N-oxides thereof.

A particularly preferred compound of the invention is 2,6-difluoro-N-[3-(4,5,6,7-tetrahydro-1H-imidazol[4,5-c]pyridin-2-yl)-1H-pyrazol-4-yl]-benzamide.

Another particularly preferred compound is 2,6-difluoro-N-[3-(1,4,6,7-tetrahydro-thiopyrano[3,4-d]imidazol-2-yl)-1H-pyrazol-4-yl]-benzamide.

Salts, Solvates, Tautomers, Isomers, N-Oxides, Esters, Prodrugs and Isotopes

A reference to a compound of the formulae (I) and sub-groups thereof also includes ionic forms, salts, solvates, isomers, tautomers, N-oxides, esters, prodrugs, isotopes and protected forms thereof, for example, as discussed below; preferably, the salts or tautomers or isomers or N-oxides or solvates thereof; and more preferably, the salts or tautomers or N-oxides or solvates thereof.

As in the preceding sections of this application, all references to formula (I) in this and the following sections should be taken to refer also to formulae (Ia), (Ib), (Ic), (II), (III) and (IV) and sub-formulae, sub-groups, embodiments, preferences and examples thereof unless the context indicates otherwise.

Many compounds of the formula (I) can exist in the form of salts, for example acid addition salts or, in certain cases salts of organic and inorganic bases such as carboxylate, sulphonate and phosphate salts. It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19. All such salts are within the scope of this invention, and references to compounds of the formula (I) include the salt forms of the compounds.

Acid addition salts may be formed with a wide variety of acids, both inorganic and organic.

Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, uidecylenic and valeric acids, as well as acylated amino acids and cation exchange resins.

One sub-group of acid addition salts includes salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

Another sub-group of salts consists of salts formed from hydrochloric, acetic, methanesulphonic, adipic, L-aspartic and DL-lactic acids.

A further sub-group of salts consists of the acetate, mesylate, ethanesulphonate, DL-lactate, adipate, D-glucuronate, D-gluconate and hydrochloride salts.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Where the compounds of the formula (I) contain an amine function, these may form quaternary ammonium salts, for example by reaction with an alkylating agent according to methods well known to the skilled person. Such quaternary ammonium compounds are within the scope of formula (I).

Compounds of the formula (I) containing an amine function may also form N-oxides. A reference herein to a compound of the formula (I) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formula may exist in a number of different geometric isomeric, and tautomeric forms and references to compounds of the formula (I) include all such forms. For the avoidance of doubt, where a compound can exist in one of several geometric isomeric or tautomeric forms and only one is specifically described or shown, all others are nevertheless embraced by formula (I).

For example, in compounds of the formulae (Ia) and (Ib), the 4-oxo-4,5-dihydro-1H-imidazo[4,5-c]pyridin-2-yl and 4-oxo-4,5-dihydro-1H-imidazo[4,5-d]pyridazin-2-yl group may take either of the following two tautomeric forms A and B. For simplicity, the general formula (Ia) illustrates form A but the formula is to be taken as embracing both tautomeric forms.

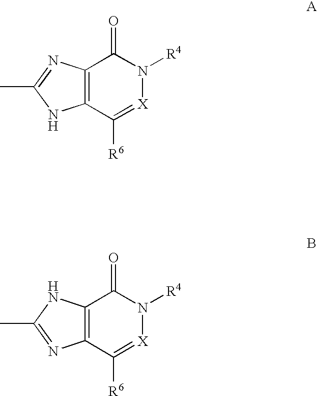

Also, in compounds of the formula (I) the 4,5,6,7-tetrahydroimidazo[4,5-c]pyridine, 1,4,6,7-tetrahydro-thiopyrano[3,4-d]imidazole, 1,4,6,7-tetrahydro-thiopyrano[3,4-d]imidazole 5-oxide or 1,4,6,7-tetrahydro-pyrano[3,4-d]imidazole group may take either of the following two tautomeric forms C and D. For simplicity, the general formula (I) illustrates form A but the formula is to be taken as embracing both tautomeric forms.

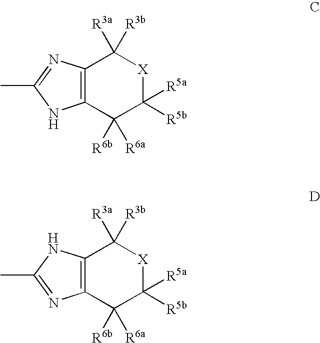

The pyrazole ring may also exhibit tautomerism and can exist in the two tautomeric forms E and F below.

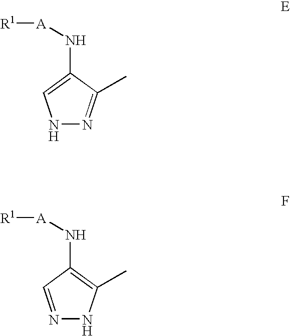

In addition, the compounds of formula (I) may exist in tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

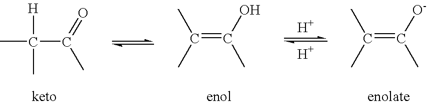

When R⁴ is not hydrogen, amide/imino alcohol tautomerism cannot occur.

Esters such as carboxylic acid esters and acyloxy esters of the compounds of formula (I) bearing a carboxylic acid group or a hydroxyl group are also embraced by Formula (I). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(—O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formula (I) are any polymorphic forms of the compounds, solvates (e.g. hydrates), complexes (e.g. inclusion complexes or clathrates with compounds such as cyclodextrins, or complexes with metals) of the compounds, and pro-drugs of the compounds and protected forms of thereof, for example, as discussed below. By “prodrugs” is meant for example any compound that is converted in vivo into a biologically active compound of the formula (I).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:
C₁₋₇alkyl
(e.g., -Me, -Et, -nPr, -iPr, -nBu, -sBu, -iBu, -tBu);
C₁₋₇aminoalkyl
(e.g., aminoethyl; 2-(N,N-diethylamino)ethyl; 2-(4-morpholino)ethyl); and acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy)carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Where compounds of the formula (I) contain one or more chiral centres, and can exist in the form of two or more optical isomers, references to compounds of the formula (I) include all optical isomeric forms thereof such as enantiomers, epimers and diastereoisomers, either as individual optical isomers, or racemic mixtures or two or more optical isomers, unless the context requires otherwise.

For example, the group R¹⁰ can include one or more chiral centres. Thus, for example, when a hydrocarbyl group has two substituents, the carbon atom to which they are attached is typically chiral and hence the compound of the formulae (I) will exist as a pair of enantiomers (or more than one pair of enantiomers where more than one chiral centre is present in the compound).

Moreover, the fused imidazole ring in formula (Ic) can include one or more chiral centres. Thus, for example, when R^{3a} and R^{3b} are both substituents, the carbon atom to which they are attached is typically chiral and hence the compound of the formula (I) will exist as a pair of enantiomers (or more than one pair of enantiomers where more than one chiral centre is present in the compound).

The optical isomers may be characterised and identified by their optical activity (i.e. as + and − isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Methods and techniques for the preparation (e.g., asymmetric synthesis) and separation (e.g., fractional crystallisation or chiral chromatography (chromatography on a chiral support)) of optical isomers are numerous and are well known to the person skilled in the art or are readily obtained by adapting the methods taught herein, or known methods, in a known manner.

Where compounds of the formula (I) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I) having one or more chiral centres, wherein at least 55% (e.g. at least 60%, 65%, 70%, 75%, 80%, 85%, 90% or 95%) of the compound of the formula (I) is present as a single optical isomer (e.g. enantiomer or diastereoisomer). Enantiomeric forms substantially free, i.e. associated with less than 5%, preferably less than 2%, in particular less than 1%, of the other enantiomeric form are also envisaged. In one general embodiment, 99% or more (e.g. substantially all) of the total amount of the compound of the formula (I) may be present as a single optical isomer (e.g. enantiomer or diastereoisomer). Unless otherwise specified, a reference to a particular compound also includes (wholly or partially) racemic forms and other mixtures thereof.

In this application, a reference to a particular chemical element includes each of its isotopes. Thus for example, hydrogen may be in any isotopic form including ¹H, ²H (deuterium) and ³H (tritium), C maybe in any isotopic form, including ¹²C, ¹³C and ¹⁴C, O may be in any isotopic form, including ¹⁶O and ¹⁸O; and the like. In one embodiment of the invention, the compounds of the formula (I) may include one or more (preferably only one or two) radioisotopes. In another embodiment of the invention, the compounds of the formula (I) contain no radioisotopes.

Biological Activity

The compounds of the formula (I) are inhibitors of cyclin dependent kinases. For example, compounds of the invention have activity against CDK1, and/or CDK2, and/or CDK3, and/or CDK4, and/or CDK5, and/or CDK6, and/or CDK7 and/or CDK8, and or CDK9 kinases.

Compounds of the invention also have activity against glycogen synthase kinase-3 (GSK-3).

Compounds of the invention also have activity against aurora kinases (e.g. aurora A kinase or aurora B kinase).

As a consequence of their activity in modulating or inhibiting CDK kinases and/or glycogen synthase kinase and/or aurora kinases, they are expected to be useful in providing a means of arresting, or recovering control of, the cell cycle in abnormally dividing cells. It is therefore anticipated that the compounds will prove useful in treating or preventing proliferative disorders such as cancers. It is also envisaged that the compounds of the invention will be useful in treating conditions such as viral infections, type II or non-insulin dependent diabetes mellitus, autoimmune diseases, head trauma, stroke, epilepsy, neurodegenerative diseases such as Alzheimer's, motor neurone disease, progressive supranuclear palsy, corticobasal degeneration and Pick's disease for example autoimmune diseases and neurodegenerative diseases.

One sub-group of disease states and conditions where it is envisaged that the compounds of the invention will be useful consists of viral infections, autoimmune diseases and neurodegenerative diseases.

CDKs play a role in the regulation of the cell cycle, apoptosis, transcription, differentiation and CNS function. Therefore, CDK inhibitors could be useful in the treatment of diseases in which there is a disorder of proliferation, apoptosis or differentiation such as cancer. In particular RB+ve and RB−ve tumours may be particularly sensitive to CDK inhibitors.

Examples of cancers which may be inhibited include, but are not limited to, a carcinoma, for example a carcinoma of the bladder, breast, colon (e.g. colorectal carcinomas such as colon adenocarcinoma and colon adenoma), kidney, epidermis, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xeroderoma pigmentosum; keratoctanthoma; thyroid follicular cancer; or Kaposi's sarcoma.

CDKs are also known to play a role in apoptosis, proliferation, differentiation and transcription and therefore CDK inhibitors could also be useful in the treatment of the following diseases other than cancer; viral infections, for example herpes virus, pox virus, Epstein-Barr virus, Sindbis virus, adenovirus, HIV, HPV, HCV and HCMV; prevention of AIDS development in HIV-infected individuals; chronic inflammatory diseases, for example systemic lupus erythematosus, autoimmune mediated glomerulonephritis, rheumatoid arthritis, psoriasis, inflammatory bowel disease, and autoimmune diabetes mellitus; cardiovascular diseases for example cardiac hypertrophy, restenosis, atherosclerosis; neurodegenerative disorders, for example Alzheimer's disease, AIDS-related dementia, Parkinson's disease, amyotropic lateral sclerosis, retinitis pigmentosa, spinal muscular atropy and cerebellar degeneration; glomerulonephritis; myelodysplastic syndromes, ischemic injury associated myocardial infarctions, stroke and reperfusion injury, arrhythmia, atherosclerosis, toxin-induced or alcohol related liver diseases, haematological diseases, for example, chronic anemia and aplastic anemia; degenerative diseases of the musculoskeletal system, for example, osteoporosis and arthritis, aspirin-sensitive rhinosinusitis, cystic fibrosis, multiple sclerosis, kidney diseases and cancer pain.

Thus, in the pharmaceutical compositions, uses or methods of this invention for treating a disease or condition comprising abnormal cell growth, the disease or condition comprising abnormal cell growth in one embodiment is a cancer.

Particular subsets of cancers include breast cancer, ovarian cancer, colon cancer, prostate cancer, oesophageal cancer, squamous cancer and non-small cell lung carcinomas.

The compounds of this invention are useful in alleviating or reducing the incidence of cancer.

In the case of compounds having activity against aurora kinase, particular examples of cancers where it is envisaged that the aurora kinase inhibiting compounds of the invention will be useful include:

human breast cancers (e.g. primary breast tumours, node-negative breast cancer, invasive duct adenocarcinomas of the breast, non-endometrioid breast cancers);

ovarian cancers (e.g. primary ovarian tumours);

pancreatic cancers;

human bladder cancers;

colorectal cancers (e.g. primary colorectal cancers);

gastric tumours;

renal cancers;

cervical cancers:

neuroblastomas;

melanomas;

lymphomas;

prostate cancers;

leukemia;

non-endometrioid endometrial carcinomas;

gliomas;

non-Hodgkin's lymphoma;

Cancers which may be particularly amenable to Aurora inhibitors include breast, bladder, colorectal, pancreatic, ovarian, non-Hodgkin's lymphoma, gliomas and nonendometrioid endometrial carcinomas.

One group of cancers includes human breast cancers (e.g. primary breast tumours, node-negative breast cancer, invasive duct adenocarcinomas of the breast, non-endometrioid breast cancers); and mantle cell lymphomas. In addition, other cancers are colorectal and endometrial cancers.

Another sub-set of cancers includes hematopoietic tumours of lymphoid lineage, for example leukemia, chronic lymphocytic leukaemia, mantle cell lymphoma and B-cell lymphoma (such as diffuse large B cell lymphoma).

One particular cancer is chronic lymphocytic leukaemia.

Another particular cancer is mantle cell lymphoma.

Another particular cancer is diffuse large B cell lymphoma.

It is further envisaged that the compounds of the invention, and in particular those compounds having aurora kinase inhibitory activity, will be particularly useful in the treatment or prevention of cancers of a type associated with or characterised by the presence of elevated levels of aurora kinases, for example the cancers referred to in this context in the introductory section of this application.

The activity of the compounds of the invention as inhibitors of cyclin dependent kinases, aurora kinases and glycogen synthase kinase-3 can be measured using the assays set forth in the examples below and the level of activity exhibited by a given compound can be defined in terms of the IC₅₀ value. Preferred compounds of the present invention are compounds having an IC₅₀ value of less than 1 micromole, more preferably less than 0.1 micromole.

Methods for the Preparation of Compounds of the Formula (I)

Compounds of the Formula (I) Wherein R^{q} is a Group (a) or (b) and Compounds of the Formulae (Ia) and (Ib)

Compounds of the formula (I) wherein R^{q} is a group (a) or (b) and compounds of the formulae (Ia) and (Ib) can be prepared in accordance with synthetic methods well known to the skilled person.

Unless stated otherwise X, R¹, R², R³, R⁴, R⁵, R⁶, R¹⁰ and R⁰ are as herein defined.

Compounds of the formula (Ia) where X is N or CR⁵ and (Ib) wherein R¹-A- forms an acyl group can be prepared as illustrated in the Schemes 1 to 6 below.

As shown in Scheme 1, an amine of the formula (X) can be reacted with a carboxylic acid, or reactive derivative thereof, of the formula R¹—B—CO₂H under standard amide formation conditions. Thus, for example, the coupling reaction between the carboxylic acid and the amine (X) can be carried out in the presence of a reagent of the type commonly used in the formation of peptide linkages. Examples of such reagents include 1,3-dicyclohexylcarbodiimide (DCC) (Sheehan et al, J. Amer. Chem Soc. 1955, 77, 1067), 1-ethyl-3-(3′-dimethylaminopropyl)-carbodiimide (EDC or EDAC or EDCI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) (L. A. Carpino, J. Amer. Chem. Soc., 1993, 115, 4397) and phosphonium-based coupling agents such as 1-benzo-triazolyloxytris(pyrrolidino)phosphonium hexafluorophosphate (PyBOP) (Castro et al, Tetrahedron Letters, 1990, 31, 205). Carbodiimide-based couling agents are advantageously used in combination with 1-hydroxybenzotriazole (HOBt) (Konig et al, Chem. Ber., 103, 708, 2024-2034). Preferred coupling reagents include EDC and DCC in combination with HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive at an appropriately elevated temperature. The reaction may be carried out in the presence of a non-interfering base, for example a tertiary amine such as triethylamine or N,N-diisopropylethylamine.

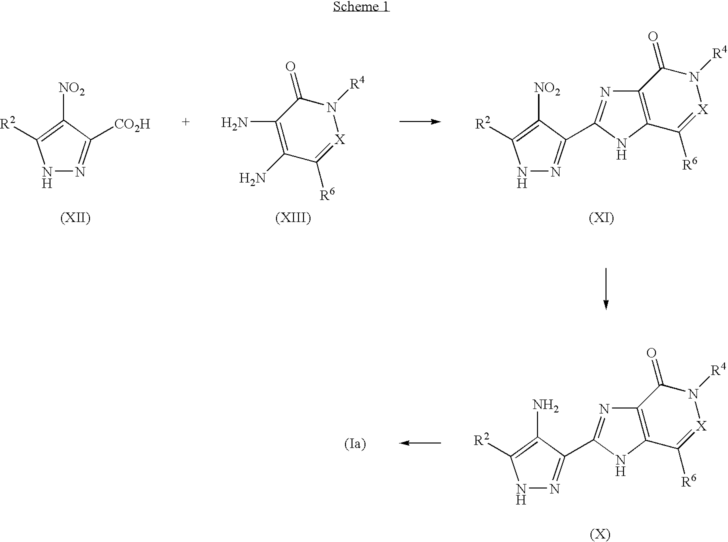

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

Amines of the formula (X) can be prepared by reduction of the corresponding nitro-compound of the formula (XI) under standard conditions. The reduction may be effected, for example, by catalytic hydrogenation in the presence of a catalyst such as palladium on carbon in a polar solvent such as ethanol or dimethylformamide at room temperature.

The compounds of the formula (XI) can be prepared by reaction of a nitro-pyrazole carboxylic acid of the formula (XII) with the appropriate diamine of the formula (XIII), for example where X is N the compounds of formula (XIII) are 4,5-diamino-2H-pyridazin-3-ones and where X is CR⁵ they are 3,4-diamino-1H-pyridin-2-ones. The cyclisation reaction between the diamine (XIII) and carboxylic acid (XII) can be carried out under amide coupling conditions as described above. For example the coupling conditions can be use of a reagent such as DCC or EDC in the presence of HOBt, which gives an intermediate (not shown) which is then cyclised reflux in the presence of acetic acid. Alternatively, the reaction of the diamine (XIII) and the acid (XII) can be carried out by heating in polyphosphoric acid in an analogous fashion to literature methods (for X═CR⁵, D. W. Robertson et. al, J. Med. Chem., 1985, 28(6), 717-727 and for X═N, DE 3347290A1)

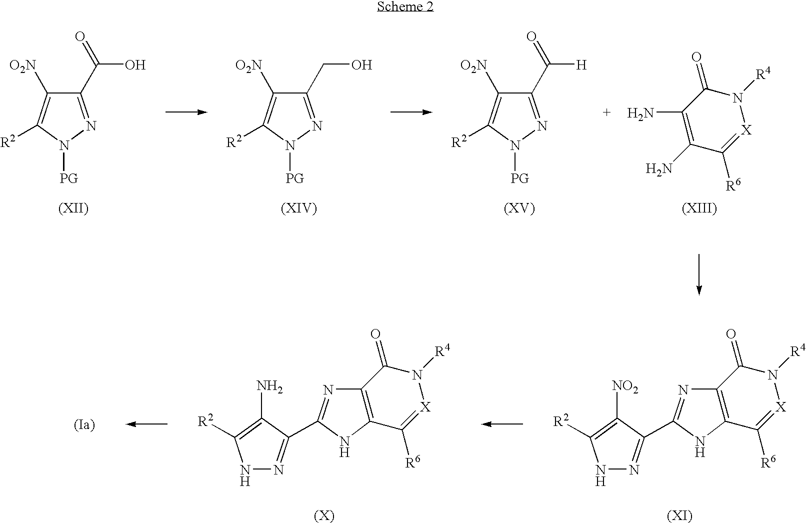

The compounds of the formula (XI) can also be prepared as outlined in Scheme 2 by reaction of a nitro-pyrazole formyl of the formula (XV) with the appropriate diamine of the formula (XIII). The cyclisation reaction between the diamine (XIII) and formyl (XV) can be performed by heating in MeNO₂ (M. Hammond et al.; Bioorg, Med. Chem. Lett., 2003, 13(12), 1989-1992), PhNO₂ (Phosphorous, Sulfur and Silicon and the Related Elements, 2001, (174), 81-92) or in DMF with an oxidising agent such as FeCl₃ (M. P. Singh et. al, Synthesis, 2000, (10), 1380-1390). For diamines of formula (XIII) where X═N, the pyrazole aldehyde can be heated with the diamine (XIII) in PhNO₂ to afford XI in an analogous way to methods described in J. Heterocyclic Chemistry, 1984, 21(5), 1249-1255.

The nitro-pyrazole formyl of the formula (XV) can be prepared from the corresponding carboxylic acid by reduction to the alcohol and then limited oxidation to the aldehyde using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995 and Handbook of Reagents for Organic Synthesis: Oxidizing and Reducing Agents, S. D. Burke, R. L. Danheiser, John Wiley and Sons Ltd, 1999. The methyl ester derivative of the carboxylic acid group of the nitro compound (XII) can be reduced to the hydroxymethyl compound (XIV) using standard techniques for example using diisobutylaluminium hydride in a non-polar solvent such as THF at −78° C. The hydroxymethyl compound is then oxidised to the formyl compound in an aprotic solvent such as acetone using manganese oxide (MnO₂). The aldehyde (XV) can also be obtained following methods similar to those found in Annali di Chimica (Rome, Italy) (1964), 54(5), 539-48.

Diamines of the formula (XIII) can be obtained commercially or can be prepared from appropriately substituted pyridinone or pyridazinone precursor compounds using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995. Examples of methods of preparing diamines of the formula (XIII) are provided in the examples below, where 4-chloro-3-nitro-2-pyridone is heated in the microwave (50 W) at 110° C. in methanolic ammonia to give 3-Amino-4-nitro-1H-pyridin-2-one which is then hydrogenated to the diamine (XIII) using 10% Pd/C in hot DMF.

In particular, diamines of formula (XIII) where X═CR⁵ can be prepared using methods analogous to those described in the patent literature (WO 00/67746) as shown in Scheme 3 below:

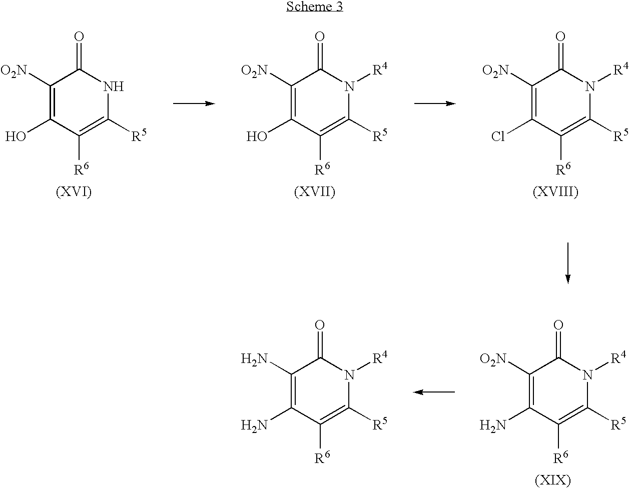

In this case compounds of formula (XVI) can be alkylated with an alkyl halide (such as for example. methyl iodide, 4-(2-chloro-ethyl)-morpholine, 4-(3-chloro-propyl)-morpholine, 4-chloromethyl-tetrahydro-pyran, or 4-chloromethyl-N—BOC-piperidine) in solvents such as DMF or NMP using a base such as NaH, to give (XVII). Conversion to (XVIII) would be afforded by treatment with POCl₃. Compounds of formula (XIX) would be afforded by heating (XVIII) in saturated methanolic ammonia at 65° C., or at temperatures ranging from 100-200° C. using a sealed tube or microwave reactor. Reduction to the diamine can be done using Pd/C, H₂, EtOH, as described elsewhere in this patent or using Zn/sat. aq, NH₄Cl/MeOH at 65° C. In the case of 4-chloromethyl-N—BOC-piperidine, the BOC-protected piperidine would require deprotection, prior to POCl₃ treatment, followed by methylation with MeI/K₂CO₃/DMF or using reductive alkylation conditions such as CH₂O/MeOH/NaBH₃CN or using CH₂O/HCO₂H/H₂O to add the R⁴ group.

In particular, diamines of formula (XIII) where X═N can also be prepared following methods analogous to those described in the literature (J. Heterocyclic Chemistry, 1984, 21(2), 481-489) as shown in Scheme 4 below:

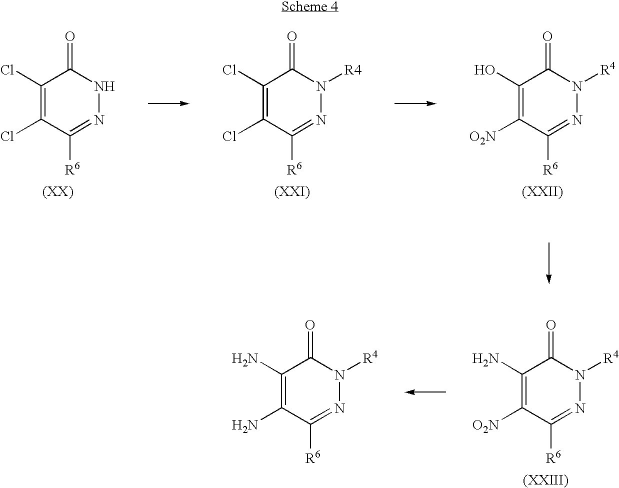

In this case compound of formula (XXI) could be afforded by alkylation of (XX) with an alkyl halide (e.g. methyl iodide, 4-(2-chloro-ethyl)-morpholine, 4-(3-chloro-propyl)-morpholine, 4-chloromethyl-tetrahydro-pyran, or 4-chloromethyl-N—BOC-piperidine) in solvent such as DMF or NMP using a base such as NaH, iPr₂EtN, Et₃N, Cs₂CO₃, at temperatures ranging from 20-100° C., depending on each case. Again, in the case of 4-chloromethyl-N—BOC-piperidine, removal of the BOC group in compound of formula (XXI) followed by methylation could be performed prior to transformation to (XXII). Compounds of formula (XX) can be obtained commercially or can be prepared from appropriately substituted pyridinone or pyridazinone precursor compounds using standard chemistry and well known functional group interconversions, see for example, Fiesers' Reagents for Organic Synthesis, and Organic Syntheses, 1995.

Compounds of formula (XXII) could then be afforded by treatment of (XXI) with NaNO₂, in aq. DMF or aq. NMP as solvent and heating to 90° C.-200° C. for 24 h, using a sealed tube where necessary. Compound of formula (XXIII) would be afforded, again following analagous procedures to those described, by treatment of a compound of formula (XXII) with sat. methanolic ammonia at 100-200° C. in a sealed tube or microwave reactor. The diamine of formula (XIII) where X is N can then be afforded by reduction with H₂/EtOH in the presence of PtO₂ or Pd/C.

The diamines of the formula (XIII) can also be reacted with carboxylic acids of the formula (XXIV) to give compounds of the formula (Ia) as shown in Scheme 4.

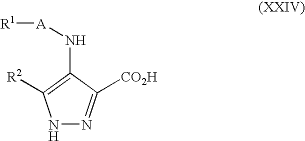

The reaction of the diamine (XIII) with the carboxylic acid (XXIV) can be carried out under conditions analogous to those described above for preparing the nitro-compounds (XI). Carboxylic acids of the formula (XXIV) can be prepared by the sequence of reactions shown in Scheme 5. As shown in Scheme 5, the amine (XXVII) is coupled with an appropriate carboxylic acid R¹—CO₂H under amide forming conditions the same as or analogous to those described above to give the amide (XXVIII). The ester group of the amide (XXVIII) can then be hydrolysed using an alkali metal hydroxide such as sodium hydroxide in a polar water miscible solvent such as methanol, typically at room temperature to give the carboxylic acid (XXIV) for use in the cyclisation reaction as described above.

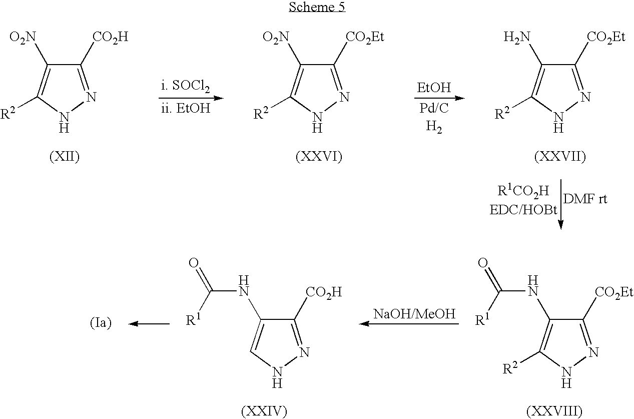

Compounds of formula (Ib) can be synthesised by use of diamine (XXIX) instead of (XIII) in the cyclisation reactions with carboxylic acids (XII) and (XV).

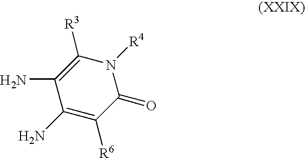

Diamines of the formula (XXIX) can be obtained commercially or can be prepared from appropriately substituted pyridinone precursor compounds using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995. Diamines of the formula (XXIX) can, for example, be synthesised by utilising the chemistry outline above for diamines of formula (XIII).

Alternatively a method based on coupling of a 2-chloro-4,5-diaminopyridine to a fully protected nitro-pyrazole-carboxylic acid could be utilized as outlined in Scheme 6. The chloropyridine moiety could then be hydrolysed to the pyridone, R⁴ being introduced by alkylation of this pyridone moiety as described previously.

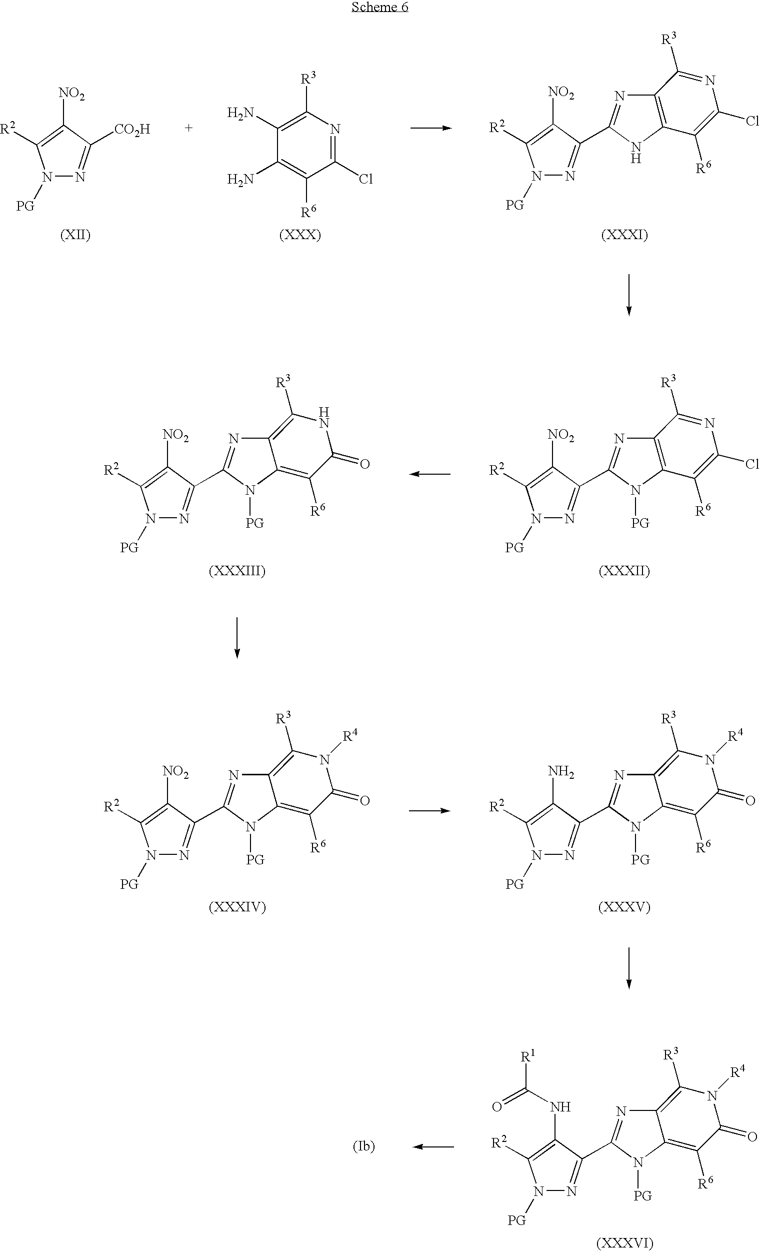

Thus, coupling the protected nitropyrazole carboxylic acid (XII) with the diamino chloropyridine (XXX) and cyclising to give (XXXI) using methods described for preparation of (XI). 2-chloro-4,5-diamino pyridine is commercially available. Similarly, compounds of formula (XXXI) could also be afforded using the protected nitropyrazole aldehyde (XV) as starting material, using methods analagous to those described for (XI). The azabenzimidazole (XXXI) would require protection, for example using a BOC (A. K. Nadipuram et. al., Org. Lett; 2002, 4(25), 4543-4546), CH₂OCH₂Ph (D. Guianvarc et. al., Tet. Lett., 2001, 42(4), 647-650), THP (J. J. Cui et. al., Bioorg. Med. Chem. Lett., 2002, 12(20), 2925-2930), or SEM (CH₂OCH₂CH₂SiMe₃) group (Tetrahedron, 1996, 52(43), 13671-13680). The pyrazole could be protected using a THP protecting group, and this can be synthesized using standard techniques.

The fully protected compound (XXXII) would then be subjected to base hydrolysis to afford the pyridone intermediate (XXXIII). This could be performed with NaOH (or NaOH/H₂O₂) in H₂O/MeOH or H₂O/dioxane following procedures described in the literature for the hydrolysis of chloropyridines (e.g. Australian J. Chem., 1984, 37(12), 2469-2477).

Alkylation of the fully protected intermediate (XXXIII) with an alkyl halide such as those outlined above using conditions previously described would afford (XXXIV). Reduction of compounds of formula (XXXIV)) using standard conditions (Pd/C, H₂) would give compounds of formula (XXXV). Amide coupling as described above and final deprotection using standard conditions, such as HCl/dioxane, TsOH/EtOH/60° C. (for THP, BOC protecting groups), Pd/H₂ (for OCH2OCH2Ph), or, TBAF or HF (for SEM protecting group) would afford the compounds of formula (Ib).

In the case of where R⁴ is introduced via reaction with a alkyl halide containing a —N—BOC protected piperidine e.g. 4-chloromethyl-N—BOC-piperidine, the BOC group could be removed during the final, deprotection step and the methyl group introduced by submitting the compound to standard methylation conditions described earlier. Alternatively, the BOC group could be removed selectively from XXXIV using TFA/CH₂Cl₂/anisole at an earlier stage (provided suitable protection group on the benzimidazole was used, (e.g. CH₂OCH₂Ph, THP or SEM), followed by methylation using standard methylating conditions.

In a reaction analogous to that described for compounds of formula (Ib) compounds of formula (Ia) where X is CR⁵ or N can be formed from the chloro-pyridine or pyridazine. As outlined in Scheme 7 the heterocycle (XXXVIII) could be prepared from the corresponding 2-chloro-3,4-diaminopyridines, for example, following the procedures outlined in (J. Heterocyclic Chemistry, 1980, 17(8) 1757-1760) or from 3-chloro-4,5-diamino-pyridazines, by using methods analogous to those described in the preparation of (XI). The resulting chloro compound (XXXVIII) could then be reacted as described above.

Other substituted 2-chloro-3,4-diaminopyridines, are described in the literature, such as where R⁶=Me (Nucleosides, Nucleotides and Nucleic acids, 2002, 21 (11+12), 737-751) and where R⁶═F (Nucleosides, Nucleotides and Nucleic acids, 2001, 20(12), 1975-2000). Other substituted 3-chloro-4,5-diamino-pyridazines are also described in the literature, e.g. where R⁶=Me (Chemical and Pharmaceutical Bulletin, 1970, 18(8), 1680-1685).

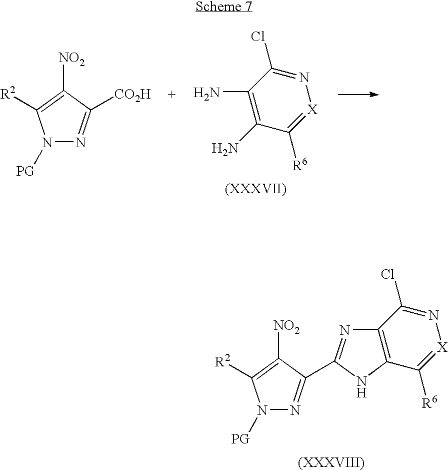

The starting material for the synthetic routes shown in Schemes 1 to 7, pyrazoles of formula (XII) or (XV), can either be obtained commercially or can be prepared by methods known to those skilled in the art. They can be obtained using known methods e.g. from ketones, such as in a process described in EP308020 (Merck), or the methods discussed by Schmidt in Helv. Chim. Acta., 1956, 39, 986-991 and Helv. Chim. Acta., 1958, 41, 306-309. Alternatively they can be obtained by conversion of a commercially available pyrazoles, for example those containing halogen, nitro, ester, or amide functionalities, to pyrazoles containing the desired functionality by standard methods known to a person skilled in the art. For example, in a 4-nitropyrazole the nitro group can be reduced to an amine by standard methods for example, reduced using palladium on carbon according to standard conditions to give the amine (XVII), 4-nitro-pyrazole-3-carboxylic acid (XII) can either be obtained commercially or can be prepared by nitration of the corresponding 4-unsubstituted pyrazole carboxy compound, and pyrazoles containing a halogen, may be utilized in coupling reactions with tin or palladium chemistry. A substituted or unsubstituted 4-amine-3-pyrazole carboxylic acid or 4-nitro-3-pyrazole carboxylic acid (XII) can be esterified by reaction with thionyl chloride to give the acid chloride intermediate followed by reaction with an alcohol to form the ester for example of formula (XVI). Alternatively, the esterification can be carried out by reacting the alcohol and carboxylic acid in the presence of an acidic catalyst, one example of which is thionyl chloride. The reaction is typically carried out at room temperature using the esterifying alcohol (e.g. ethanol) as the solvent.

Compounds of the formulas (Ia) and (Ib) in which A is NH(CO) can be prepared using the Schemes described previously using standard methods for the synthesis of ureas. For example, such compounds can be prepared by reacting an aminopyrazole compound of the formula (X) or (XVII) with a suitably substituted phenylisocyanate in a polar solvent such as DMF. The reaction is conveniently carried out at room temperature.

Compounds of the formulas (Ia) and (Ib) in which A is bond can be prepared using the Schemes described previously using standard methods for the synthesis of secondary and tertiary amines. For example, such compounds can be prepared by reacting an aminopyrazole compound of the formula (X), (XXVII), (XXXV) or (XXXIX) with a suitably substituted alkylating agent in a polar solvent such as DMF. The reaction is conveniently carried out at room temperature.

Once formed, one compound of the formulae (Ia) or (Ib) may be transformed into another compound of the formulae (Ia) or (Ib) using standard chemistry procedures well known in the art. For examples of functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995. For example, they can be prepared by converting one compound of the formulas (Ia) or (Ib) into another compound of the formulas (Ia) or (Ib) by reacting with alkylating agents, sulphonyl chlorides or acyl chlorides using methods known to a person skilled in the art. In particular these methods can be used to introduce a group R⁴ onto the nitrogen once any protecting group present has been removed. In the reactions described in Schemes 1 to 7, the group R⁴ may be replaced with a protecting group in diamines (XIII) and (XIV). The protecting group is removed from final compounds to allow introduction of the group R⁴ using some of the reactions outlined above.

Compounds of formula (Ia) (where X═CH or N) or (Ib) can be further elaborated to introduce a group R⁴ by the following known procedures. Once the 4-oxo-4,5-dihydro-1H-imidazo[4,5-c]pyridin-2-yl, 4-oxo-4,5-dihydro-1H-imidazo[4,5-d]pyridazin-2-yl or 6-oxo-5,6-dihydro-1H-imidazo[4,5-c]pyridin-2-yl core has been synthesised using the methods outlined in Scheme 1 to 6, further transformations of these compounds to add R⁴ can follow two alternative routes, as outlined in Scheme 8, depending on the nature of the electrophile used to introduce R⁴ and the specific hetero-bicyclic system concerned. Alkylation of the pyridone/pyridazinone using an alkyl halide (e.g. such as for example. methyl iodide, 4-(2-chloro-ethyl)-morpholine, 4-(3-chloro-propyl)-morpholine, 4-chloromethyl-tetrahydro-pyran, or 4-chloromethyl-N—BOC-piperidine) in DMF or NMP using a base such as iPr₂EtN, Et₃N, Cs₂CO₃, or NaH, at temperatures ranging from 20 to 100° C., depending on the reagents obtains compounds of formula (XI). Reduction of the nitro group, formation of the amide would follow routes previously described above. Final deprotection of the protecting groups will afford the compound of formula (Ia).

Depending on the relative reactivity of the different nucleophilic centres and the nature of the electrophile, to introduce R⁴, initial protection of the fused imidazole could be employed to give (XXXIX). Protecting groups as described above could be used (e.g. THP, BOC, CH₂OCH₂Ph, SEM) and then the alkylation of the pyridone/pyridazinone, as for (XI) can occur. Further elaboration of the pyrazole and final deprotection would be afforded in an analogous fashion to (XI) and (XXXVI) respectively. Similar processes can be followed for compounds of formula (Ib).

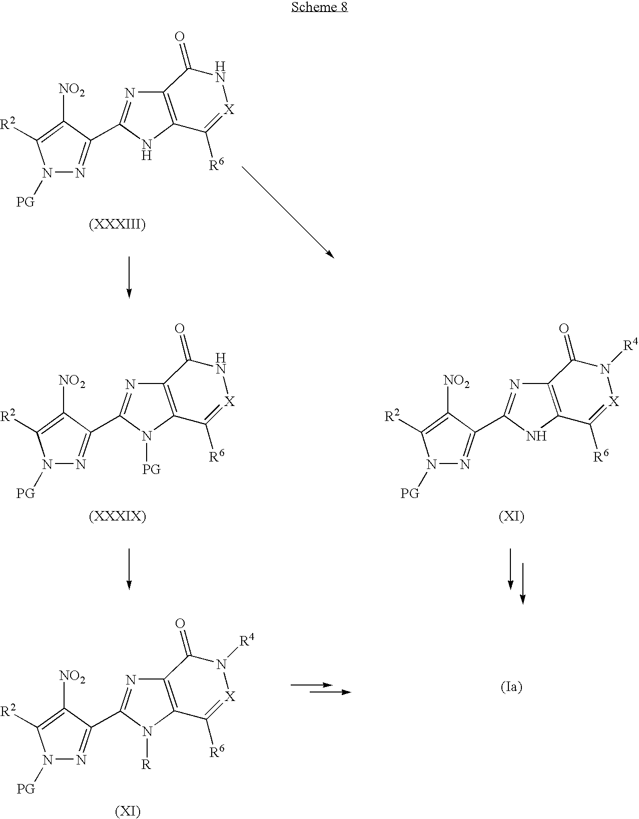

In the case of an N—BOC protected piperidine, the BOC group would be removed in the final deprotection step. The methyl group could be introduced via MeI/K₂CO₃ or other methylation conditions such as CH₂O/H₂/Pd—C or HCO₂H/CH₂O/H₂O. Alternatively, the BOC group could be removed (TFA/CH₂Cl₂/anisole) at an earlier stage, and the piperidine methylated, and then the synthesis continued as described. The BOC group can be removed selectively in the presence of other protecting groups for example, THP, by using TFA/CH₂Cl₂/anisole.

Compounds of the Formula (I) Wherein R^{q} is a Group (c) and Compounds of the Formula (Ic)

Compounds of the formula (I) wherein R^{q} is a group (c) and compounds of the formula (Ic) can be prepared in accordance with synthetic methods well known to the skilled person.

Unless stated otherwise X″, R¹, R², R^{3a}, R^{3b}, R^{4′}, R^{5a}, R^{5b}, R^{6a}, R^{6b}, R¹⁰ and R⁰ are as herein defined.

Compounds of the formula (I) where X″ is NR^{4′}, O, S or S(O) and wherein R¹-A-forms an acyl group can be prepared as illustrated in Schemes 9, 10, or 11 below.

As shown in Scheme 9, an amine of the formula (CX) can be reacted with a carboxylic acid, or reactive derivative thereof, of the formula R¹—B—CO₂H under standard amide formation conditions under the conditions set out and described above in connection with Scheme 1.

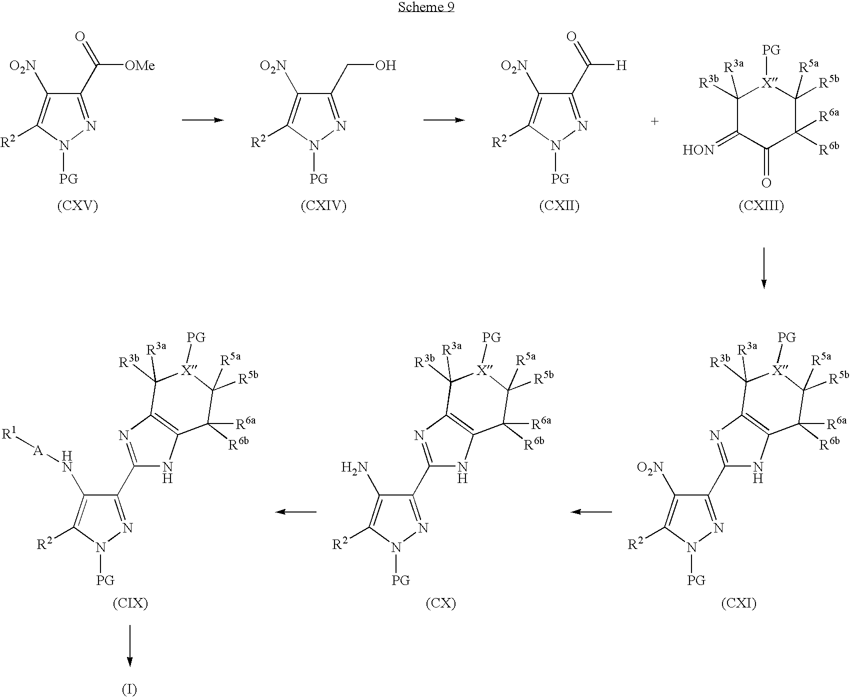

As an alternative, a reactive derivative of the carboxylic acid, e.g. an anhydride or acid chloride, may be used. Reaction with a reactive derivative such an anhydride is typically accomplished by stirring the amine and anhydride at room temperature in the presence of a base such as pyridine.

Amines of the formula (CX) can be prepared by reduction of the corresponding nitro-compound of the formula (CXI) under standard conditions. The reduction may be effected according to standard methods, for example by catalytic hydrogenation in the presence of a catalyst such as palladium on carbon in a polar solvent such as ethanol or dimethylformamide at room temperature.

The compounds of the formula (CXI) can be prepared by reaction of a nitro-pyrazole formyl of the formula (CXII) with the appropriate saturated heterocycle of the formula (CXIII), for example where X″ is N the compounds of formula (CXIII) are 3-hydroxyamino-4-oxo-piperidines. The cyclisation reaction between the saturated 3-hydroxyamino heterocycle (CXIII) and formyl compound (CXII) can be carried out in the presence of a reagent such as ammonium acetate or ammonia. This cyclisation step is typically carried out by heating under reflux in the presence of acetic acid.

The nitro-pyrazole formyl of the formula (CXII) can be prepared from the corresponding carboxylic acid, or methyl ester derivative, by reduction to the alcohol and then limited oxidation to the aldehyde using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995 and Handbook of Reagents for Organic Synthesis Oxidizing and Reducing Agents, S. D. Burke, R. L. Danheiser, John Wiley and Sons Ltd, 1999. The methyl ester derivative of the carboxylic acid group of the nitro compound (CXV) can be reduced to the hydroxymethyl compound (CXIV) using standard techniques for example using diisobutyl-aluminium hydride in a non-polar solvent such as THF at −78° C. The hydroxymethyl compound is then oxidised to the formyl compound in an aprotic solvent such as acetone using manganese oxide (MnO₂).

Where X″ is N, the saturated heterocycle of formula (CXIII) is a 3-hydroxyamino-4-oxo-piperidine. 3-Hydroxyamino-4-oxo-piperidines can be prepared using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995. An example of a method of preparing 3-hydroxyamino-4-oxo-piperidines of the formula (CXIII) is provided in the examples below, whereby the appropriate 4-oxo-piperidine is reacted with TMSCl and isoamyl nitrate at room temperature for 30 minutes. Alternatively 3-hydroxyamino-4-oxo-piperidines could be prepared by reacting the 4-oxo-piperidine with sodium nitrite and acetic acid.

Appropriately substituted 4-oxo-piperidine precursor compounds can be obtained commercially or can be prepared from using standard chemistry and well known functional group interconversions, see for example, Fiesers' Reagents for Organic Synthesis. It may be necessary that a protecting group be present on the piperidine ring nitrogen for this and/or subsequent reactions, and appropriate protecting groups for this purpose are discussed below.

Where X″ is O or S, the compound of formula (CXIII) are 3-hydroxyamino-4-pyranones or 3-hydroxyamino-4-thiopyranones, respectively. These can be prepared from the appropriately substituted 4-pyranone or 4-thiopyranone precursor compounds using standard chemistry and well known functional group interconversions, examples of which are provided above. Appropriately substituted 4-pyranone or 4-thiopyranone precursor compounds can be obtained commercially or can be prepared from using standard chemistry and well known functional group interconversions, see for example, Fiesers' Reagents for Organic Synthesis. The protecting group on the heteroatom X″ need not be present where X″ is O or S.

Compounds of formula (I) where X″ is S(O) can be prepared from compounds (CXI) of Scheme 1, as illustrated in Scheme 10.

As shown in Scheme 10, the compounds of the formula (I) where X″ is S(O) can be prepared by limited oxidation of a thioether of the formula (CXI) with an oxidizing agent such as hydrogen peroxide or a per-acid (e.g. a peroxycarboxylic acid), see for example Advanced Organic Chemistry, by Jerry March, 4^{th} Edition, Wiley Interscience, pages 1201-1202. The nitro groups of the thiopyran-1-oxide of formula (CVIII) can then be reduced to the amine (CVII) and then reacted with a carboxylic acid, or reactive derivative thereof, of the formula R¹—B—CO₂H under standard amide formation conditions as discussed above and below, to give compounds of formula (I).

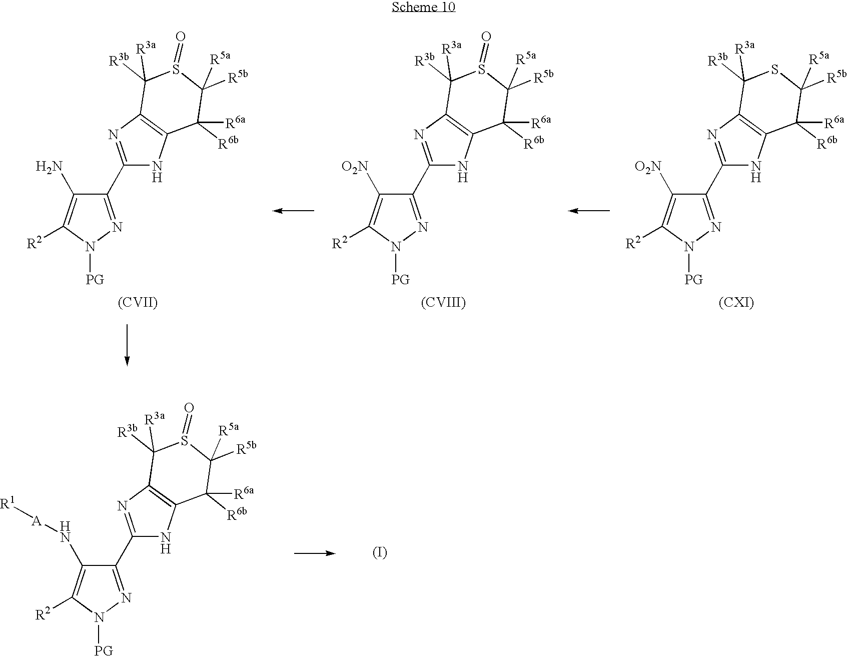

Alternatively, 3-hydroxyamino-4-oxo-piperidines, pyranones or thiopyranones of the formula (CXIII) can also be reacted with formyl compounds of the formula (CXVI) to give compounds of the formula (I) as shown in Scheme 11. The reaction of the heterocycle (CXIII) with the formyl compound (CXVI) can be carried out under conditions analogous to those described above for preparing the nitro-compounds (CXI). Formyl compounds of the formula (CXVI) can be prepared by the sequence of reactions shown in Scheme 11.

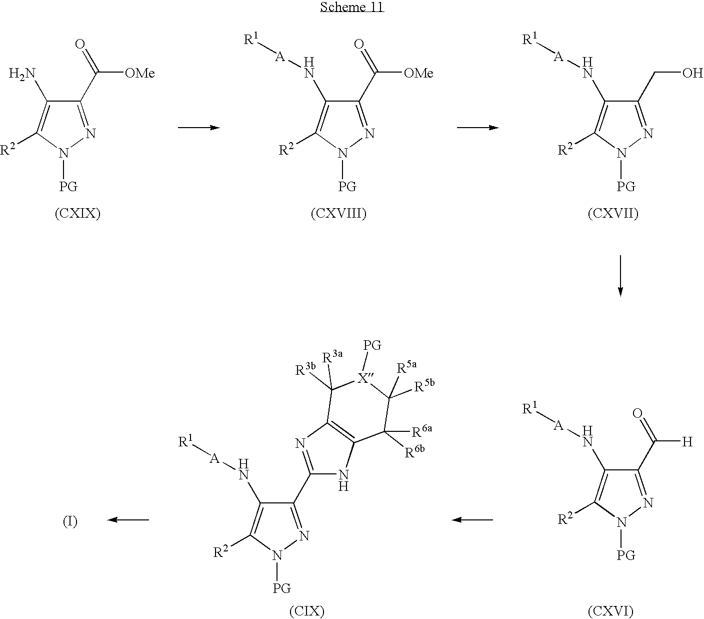

As shown in Scheme 11, the amine (CXIX) is coupled with an appropriate carboxylic acid R¹—CO₂H under amide forming conditions the same as or analogous to those described above to give the amide (CXVIII). The carboxylic ester group of the amide (CXVIII) can then be reduced to the hydroxymethyl compound (CXVII) using standard techniques for example using diisobutylaluminium hydride in a non-polar solvent such as THF at −78° C. The hydroxymethyl compound is then oxidised to the formyl compound in an aprotic solvent such as acetone using manganese oxide (MnO₂). Other reduction and oxidation reagents for performing this transformation are well known to a person skilled in the art.

A further synthetic route to compounds of Formula (I) is shown in Scheme 12. The procedure illustrated in Scheme 12 is of particular utility in the preparation of compounds when X″ is nitrogen and when, for example, R^{3b}, R^{5b} and R^{6b} are all hydrogen.

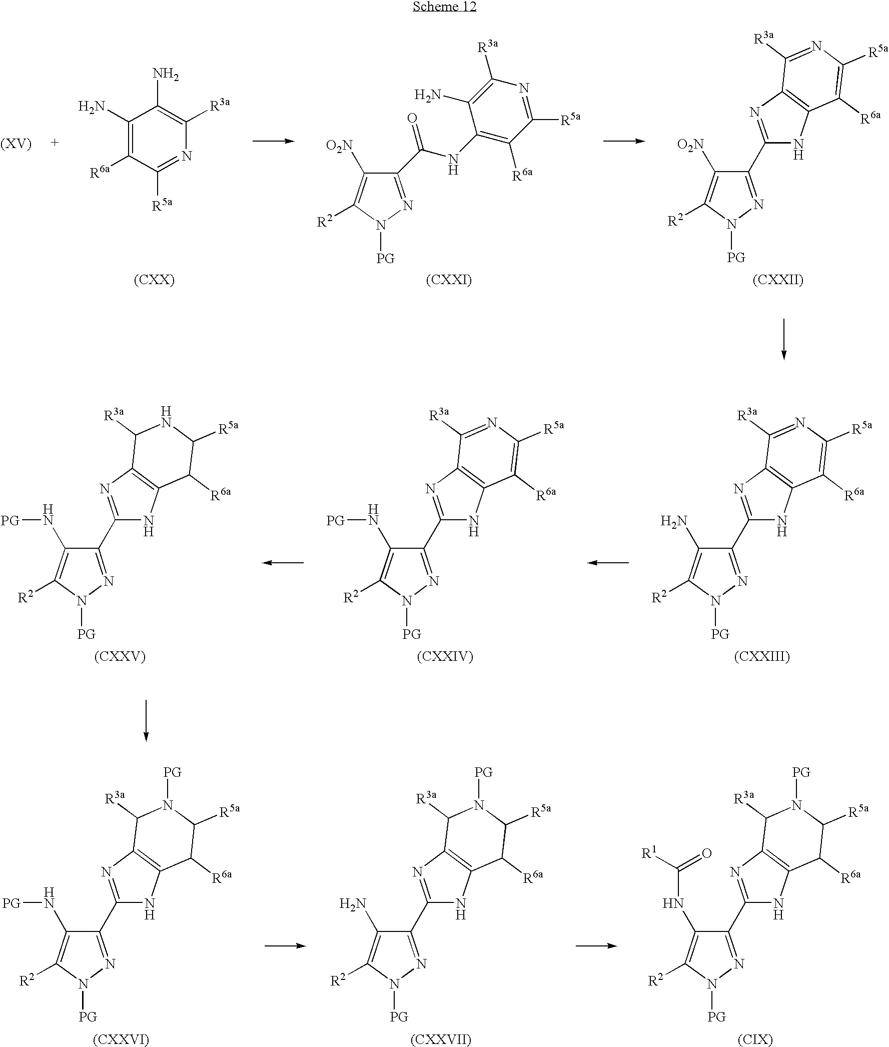

The compounds of the formula (CXXII) can be prepared by reaction of a nitro-pyrazole carboxylic acid of the formula (CXV) with a diamine of the formula (CXX). The reaction between the diamine (CXX) and carboxylic acid (CXV) can be carried out in the presence of a reagent such as DCC or EDC in the presence of HOBt as described above, under amide coupling conditions as described previously, to give an intermediate ortho-aminophenylamide (CXII) which is then cyclised to form the ring. The final cyclisation step is typically carried out by heating under reflux in the presence of acetic acid.

Diamines of the formula (CXX) can be obtained commercially or can be prepared from appropriately substituted phenyl precursor compounds using standard chemistry and well known functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995.

Amines of the formula (CXXIII) can be prepared by reduction of the corresponding nitro-compound of the formula (CXIII) under standard conditions, as described above for Schemes 9 to 11.

Tetrahydropyridines of the formula (CXXV) can be prepared by reduction of the corresponding imidazo-pyridine compound of the formula (CXXIV) under standard conditions. The reduction may be effected, for example, by catalytic hydrogenation in the presence of a catalyst such as platinum oxide in a protic solvent such as acetic acid or trifluoroacetic acid at room temperature or higher pressures.

As described herein, an amine (CXXVII) can then be reacted with a carboxylic acid, or reactive derivative thereof, of the formula R⁵—CO₂H under standard amide formation conditions, and any protecting groups removed, to give a compound of formula (Ic).

The starting materials for the synthetic routes shown in the Schemes above, pyrazoles of Formula (CXIX) and (CXV), can either be obtained commercially or can be prepared by methods known to those skilled in the art. They can be obtained using known methods e.g. from ketones, such as in a process described in EP308020 (Merck), or the methods discussed by Schmidt in Helv. Chim. Acta., 1956, 39, 986-991 and Helv. Chim. Acta., 1958, 41, 306-309. Alternatively they can be obtained by conversion of a commercially available pyrazole, for example those containing halogen, nitro, ester, or amide functionalities, to pyrazoles containing the desired functionality by standard methods known to a person skilled in the art. For example, in 3-carboxy-4-nitropyrazole the nitro group can be reduced to an amine by standard methods, 4-nitro-pyrazole-3-carboxylic acid (CXV) can either be obtained commercially or can be prepared by nitration of the corresponding 4-unsubstituted pyrazole carboxy compound, and pyrazoles containing a halogen, may be utilized in coupling reactions with tin or palladium chemistry. A substituted or unsubstituted 4-nitro-3-pyrazole carboxylic acid can be esterified by reaction with thionyl chloride to give the acid chloride intermediate followed by reaction with an alcohol to form the ester of formula (CXIX). Alternatively, the esterification can be carried out by reacting the alcohol and carboxylic acid in the presence of an acidic catalyst, one example of which is thionyl chloride. The reaction is typically carried out at room temperature using the esterifying alcohol (e.g. ethanol) as the solvent.

Compounds of the formula (Ic) in which A is NH(CO) can be prepared using the Schemes described previously using standard methods for the synthesis of ureas. For example, such compounds can be prepared by reacting an aminopyrazole compound of the formula (CX) or (CXIX) or (CXXVII) with a suitably substituted phenylisocyanate in a polar solvent such as DMF. The reaction is conveniently carried out at room temperature.

Compounds of the formula (Ic) in which A is a bond can be prepared using the Schemes described previously using standard methods for the synthesis of secondary and tertiary amines. For example, such compounds can be prepared by reacting an aminopyrazole compound of the formula (CX) or (CXIX) with a suitably substituted alkylating agent in a polar solvent such as DMF. The reaction is conveniently carried out at room temperature.

Once formed, one compound of the formula (I) may be transformed into another compound of the formula (I) using standard chemistry procedures well known in the art. For examples of functional group interconversions, see for example, Fiesers' Reagents for Organic Synthesis, Volumes 1-17, John Wiley, edited by Mary Fieser (ISBN: 0-471-58283-2), and Organic Syntheses, Volumes 1-8, John Wiley, edited by Jeremiah P. Freeman (ISBN: 0-471-31192-8), 1995. For example, they can be prepared by converting one compound of the formula (Ic) into another compound of the formula (Ic) by reacting with alkylating agents, sulphonyl chlorides or acyl chlorides using methods known to a person skilled in the art as shown in Scheme 5. In particular these methods can be used to introduce a group R^{4′} onto the nitrogen where X″ is N once any protecting group present has been removed, for example, from compounds of formula (CXI) or (CX) of Scheme 9 or compound of formula (CIX) as shown in Scheme 13.

Reductive alkylation is one particular method of introducing the group R^{4′}. Alternatively, R^{4′} can be added to compounds by alkylation with an alkyl halide (such as, for example, methyl iodide, 4-(2-chloro-ethyl)-morpholine, 4-(3-chloro-propyl)-morpholine, 4-chloromethyl-tetrahydro-pyran, or 4-chloromethyl-N—BOC-piperidine) in solvents such as DMF or NMP using a base such as iPr₂EtN, Et₃N, Cs₂CO₃, or NaH, at temperatures ranging from 20-100° C., depending on the reagents. In the case of where R^{4′} is introduced via reaction with a alkyl halide containing a —N—BOC protected piperidine e.g. 4-chloromethyl-N—BOC-piperidine, the BOC group, could be removed during the final deprotection step and an alkyl group introduced onto the piperidine nitrogen by submitting the compound to standard methylation conditions such as reaction with MeI/K₂CO₃/DMF or by using reductive alkylation conditions such as CH₂O/MeOH/NaBH₃CN or using CH₂O/HCO₂H/H₂O. Alternatively, the BOC group could be removed selectively using TFA/CH₂Cl₂/anisole at an earlier stage (provided suitable protection groups are used elsewhere in the molecule, e.g. CH₂OCH₂Ph), followed by methylation using standard methylating conditions.

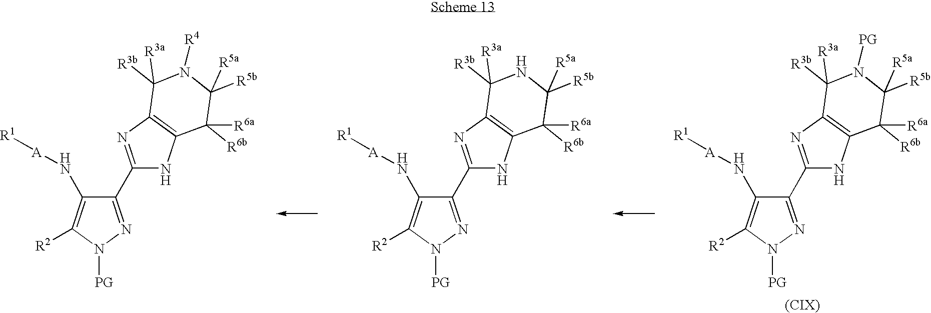

Alternatively piperidines of formula (CXIII) can be used where the protecting group is replaced with a group R^{4′}, which can then be introduced as outlined above, or R^{4′} could be present in the commercially available precursor used to synthesise the 3-hydroxyamino-4-oxo-piperidines.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999). In these circumstances a protected form of a compound of formula (I) may be produced by the procedures described above. In this case a deprotection step may be required to produce the final compound of formula (I). Deprotection of the protected form of a compound of formula (I) can be achieved using standard methods well know to those skilled in the art. Protecting groups and deprotection methods may be selected from standard groups and methods known in the art as discussed below.

A hydroxy group may be protected, for example, as an ether (—OR) or an ester (—OC(═O)R), for example, as: a t-butyl ether; a benzyl, benzhydryl (diphenylmethyl), or trityl(triphenylmethyl)ether; a trimethylsilyl or t-butyldimethylsilyl ether; or an acetyl ester (—OC(═O)CH₃, —OAc). An aldehyde or ketone group may be protected, for example, as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid. An amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulfonyl (tosyl) and methanesulfonyl (mesyl) groups and benzyl groups such as a para-methoxybenzyl (PMB) group, or tetrahydropyran (THP). A carboxylic acid group may be protected as an ester for example, as: an C₁₋₇ alkyl ester (e.g., a methyl ester; a t-butyl ester); a C₁₋₇haloalkyl ester (e.g., a C₁₋₇ trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃). In some circumstances one of the protecting groups described above may form part of the compound of Formula (I).

The present invention further includes novel chemical intermediates as described herein and in particular novel chemical intermediates of the formulae (XXIV), (XIX), (XVIII), (XVI), (XV), (XIV), (XIII), (XII), (XI), (CXVIII), (CXIX), (CXVII), (CXVI), (CXIV), (CXIII), (CXII), (CVII), (CVIII), (CXI), (CX), and (CIX). In particular, preferred novel intermediates are compounds of formulae (X) (XXIV), (CIX) or (CX), more preferably (X) and salts thereof and (CIX) and salts, solvates, esters or N-oxides thereof.

In a further aspect, the invention provides a process for the preparation of a compound of formula (I) as defined herein, which process comprises:
(i) the reaction of a compound of the formula (X) with a carboxylic acid of formula R¹—CO₂H and thereafter removing any protecting groups present; or 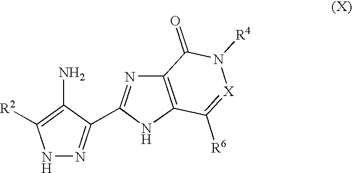
(ii) the reaction of a compound of the formula (Ia): 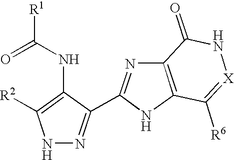
   or an appropriately protected form thereof, with an alkylating agent and thereafter removing any protecting groups present;
   wherein R¹, R², R⁴, R⁵, and R⁶ are as defined herein; and optionally thereafter converting one compound of the formula (Ia) into another compound of the formula (Ia);
(iii) the reaction of a compound of the formula (CIX) to remove any protecting groups present; or 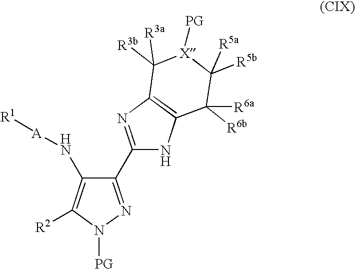
(iv) the reaction of a compound of the formula (I) where X″ is NR^{4′} and R^{4′} is hydrogen: 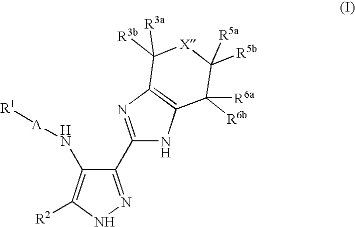
   or an appropriately protected form thereof, with an alkylating agent and thereafter removing any protecting groups present;
   wherein R¹, R², R^{3a}, R^{3b}, R^{5a}, R^{5b}, R^{6a} and R^{6b} are as defined herein; and optionally thereafter converting one compound of the formula (I) into another compound of the formula (I).
   Pharmaceutical Formulations

While it is possible for the active compound to be administered alone, it is preferable to present it as a pharmaceutical composition (e.g. formulation) comprising at least one active compound of the formula (I) as defined herein together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy. Particular examples of such agents include anti-emetic agents and agents that prevent or decrease the duration of chemotherapy-associated neutropenia and prevent complications that arise from reduced levels of red blood cells or white blood cells, for example erythropoietin (EPO), granulocyte macrophage-colony stimulating factor (GM-CSF), and granulocyte-colony stimulating factor (G-CSF).

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The invention also provides compounds of the formula (I) as hereinbefore defined in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKₐ is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

The pharmaceutical formulation can be prepared by lyophilising a compound of Formula (I) or acid addition salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastrointestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

The compounds of formula (I) will generally be presented in unit dosage form and, as such, will typically contain sufficient compound to provide a desired level of biological activity. For example, a formulation intended for oral administration may contain from 0.1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 grain, for example 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect.

Methods of Diagnosis and Treatment

It is envisaged that the compounds of the formula (I) will useful in the prophylaxis or treatment of a range of disease states or conditions mediated by cyclin dependent kinases or aurora kinases or glycogen synthase kinase. Examples of such disease states and conditions are set out above.

Compounds of the formula (I) are generally administered to a subject in need of such administration, for example a human or animal patient, preferably a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a pulsatile or continuous manner.

A typical daily dose of the compound can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example milligrams per kilogram of bodyweight although higher or lower doses may be administered where required.

Ultimately, however, the quantity of compound administered will be commensurate with the nature of the disease or physiological condition being treated and will be at the discretion of the physician.

The compounds of the formula (I) can be administered as the sole therapeutic agent or they can be administered in combination therapy with one of more other compounds for treatment of a particular disease state, for example a neoplastic disease such as a cancer as hereinbefore defined. Examples of other therapeutic agents that may be administered together (whether concurrently or at different time intervals) with the compounds of the formula (I) include cytotoxic agents, agents that prevent cell proliferation or radiotherapy. Examples of such agents include but are not limited to topoisomerase inhibitors, alkylating agents, antimetabolites, DNA binders, signal transduction inhibitors, monoclonal antibodies, and tubulin targeting agents (microtubule inhibitors), such as cisplatin, cyclophosphamide, doxorubicin, irinotecan, fludarabine, 5FU, taxanes and mitomycin C, or radiotherapy. For the case of CDK inhibitors combined with other therapies the two, three, four or more treatments may be given in individually varying dose schedules and via different routes.

It has also been discovered that some cyclin-dependent kinase inhibitors can be used in combination with other anticancer agents. For example, the cytotoxic activity of cyclin-dependent kinase inhibitor flavopiridol, has been used with other anticancer agents in combination therapy.

Where the compound of the formula (I) is administered in combination therapy with one, two, three, four or more other therapeutic agents (preferably one or two, preferably one), the compounds can be administered simultaneously (either in the same or different pharmaceutical formulation) or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

For use in combination therapy with another chemotherapeutic agent, the compound of the formula (I) and one, two, three, four or more other therapeutic agents can be, for example, formulated together in a dosage form containing two, three, four or more therapeutic agents. In an alternative, the individual therapeutic agents may be formulated separately and presented together in the form of a kit, optionally with instructions for their use.

The combination of the agents listed above with a compound of the present invention would be at the discretion of the physician who would select dosages using his common general knowledge, and dosing regimes known to skilled practitioner.

Prior to administration of a compound of the formula (I), a patient may be screened to determine whether a disease or condition from which the patient is or may be suffering is one which would be susceptible to treatment with a compound having activity against aurora kinases. For example, a biological sample taken from a patient may be analysed to determine whether a condition or disease, such as cancer, that the patient is or may be suffering from is one which is characterised by upregulation of aurora kinase, this includes elevated expression or over-expression of aurora kinase, including gene amplification (i.e. multiple gene copies) and increased expression by a transcriptional effect, and hyperactivity and activation of aurora kinase, including activation by mutations. Thus, the patient may be subjected to a diagnostic test to detect a marker characteristic of up-regulation of aurora kinase. The term diagnosis includes screening. By marker we include genetic markers including, for example, the measurement of DNA composition to identify mutations of aurora or CDC4. The term marker also includes markers which are characteristic of up regulation of aurora or cyclin E, including enzyme activity, enzyme levels, enzyme state (e.g. phosphorylated or not) and mRNA levels of the aforementioned proteins.

The diagnostic tests are typically conducted on a biological sample selected from tumour biopsy samples, blood samples (isolation and enrichment of shed tumour cells), stool biopsies, sputum, chromosome analysis, pleural fluid, peritoneal fluid, or urine.

It has been found, see Ewart-Toland et al., (Nat Genet. 2003 August; 34(4):403-12), that individuals forming part of the sub-population possessing the Ile31 variant of the STK gene (the gene for aurora kinase A) may have an increased susceptibility to certain forms of cancer. It is envisaged therefore that such individuals suffering from cancer will benefit from the administration of compounds having aurora kinase inhibiting activity. A patient suffering from, or suspected of suffering from, a cancer may therefore be screened to determine whether he or she forms part of the Ile31 variant sub-population. The screening process will typically involve direct sequencing, oligonucleotide microarray analysis, or a mutant specific antibody.

Tumours with activating mutants of Aurora or up-regulation of Aurora including any of the isoforms thereof, may be particularly sensitive to Aurora inhibitors. Tumours may preferentially be screened for up-regulation of aurora or for aurora possessing the Ile31 variant prior to treatment (Ewart-Toland et al., Nat Genet. 2003 August; 34(4):403-12). Ewart-Toland et al identified a common genetic variant in STK15 (resulting in the amino acid substitution F31I) that is preferentially amplified and associated with the degree of aneuploidy in human colon tumors. These results are consistent with an important role for the Ile31 variant of STK15 in human cancer susceptibility.

The aurora A gene maps to the chromosome 20q13 region that is frequently amplified in many cancers e.g breast, bladder, colon, ovarian, pancreatic. Patients with a tumour that has this gene amplification might be particularly sensitive to treatments targeting aurira kinase inhibition.

Methods of identification and analysis of Aurora mutations and up-regulation of Aurora isoforms and chromosome 20q13 amplification are known to a person skilled in the art. Screening methods could include, but are not limited to, standard methods such as reverse-transcriptase polymerase chain reaction (RT-PCR) or in-situ hybridisation.

In screening by RT-PCR, the level of aurora mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3^{rd} Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing aurora mRNA expression would be fluorescence in-situ hybridisation (FISH) (see Angerer, 1987 Meth. Enzymol., 152: 649).

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Alternatively, the protein products expressed from the mRNAs may be assayed by immunohistochemistry of tumour samples, solid phase immunoassay with microtiter plates, Western blotting, 2-dimensional SDS-polyacrylamide gel electrophoresis, ELISA, flow cytometry and other methods known in the art for detection of specific proteins. Detection methods would include the use of site specific antibodies. The skilled person will recognize that all such well-known techniques for detection of aurora up-regulation and mutants of Aurora could be applicable in the present case.

In addition, all of these techniques could also be used to identify tumours particularly suitable for treatment with CDK inhibitors. Tumours with mutants of CDC4 or up-regulation, in particular over-expression, of cyclin E or loss of p21 or p27 may be particularly sensitive to CDK inhibitors. Tumours may preferentially be screened for up-regulation, in particular over-expression, of cyclin E (Harwell R M, Mull B B, Porter D C, Keyomarsi K.; J Biol Chem. 2004 Mar. 26; 279(13):12695-705) or loss of p21 or p27 or for CDC4 variants prior to treatment (Rajagopalan H, Jallepalli P V, Rago C, Velculescu V E, Kinzler K W, Vogelstein B, Lengauer C.; Nature. 2004 Mar. 4; 428(6978):77-81).

Patients with mantle cell lymphoma (MCL) could be selected for treatment with a compound of the invention using diagnostic tests outlined herein. MCL is a distinct clinicopathologic entity of non-Hodgkin's lymphoma, characterized by proliferation of small to medium-sized lymphocytes with co-expression of CD5 and CD20, an aggressive and incurable clinical course, and frequent t(11;14)(q13;q32) translocation. Over-expression of cyclin D1 mRNA, found in mantle cell lymphoma (MCL), is a critical diagnostic marker. Yatabe et al (Blood. 2000 Apr. 1; 95(7):2253-61) proposed that cyclin D1-positivity should be included as one of the standard criteria for MCL, and that innovative therapies for this incurable disease should be explored on the basis of the new criteria. Jones et al (J Mol Diagn. 2004 May; 6(2):84-9) developed a real-time, quantitative, reverse transcription PCR assay for cyclin D1 (CCND1) expression to aid in the diagnosis of mantle cell lymphoma (MCL). Howe et al (Clin Chem. 2004 January; 50(1):80-7) used real-time quantitative RT-PCR to evaluate cyclin D1 mRNA expression and found that quantitative RT-PCR for cyclin D1 mRNA normalized to CD19 mRNA can be used in the diagnosis of MCL in blood, marrow, and tissue. Alternatively, patients with breast cancer could be selected for treatment with a CDK inhibitor using diagnostic tests outline above. Tumour cells commonly overexpress cyclin E and it has been shown that cyclin E is over-expressed in breast cancer (Harwell et al, Cancer Res, 2000, 60, 481-489). Therefore breast cancer may in particular be treated with a CDK inhibitor as provided herein.

Antifungal Use

In a further aspect, the invention provides the use of the compounds of the formula (I) as hereinbefore defined as antifungal agents.

The compounds of the formula (I) may be used in animal medicine (for example in the treatment of mammals such as humans), or in the treatment of plants (e.g. in agriculture and horticulture), or as general antifungal agents, for example as preservatives and disinfectants.

In one embodiment, the invention provides a compound of the formula (I) as hereinbefore defined for use in the prophylaxis or treatment of a fungal infection in a mammal such as a human.

Also provided is the use of a compound of the formula (I) for the manufacture of a medicament for use in the prophylaxis or treatment of a fungal infection in a mammal such as a human.

For example, compounds of the invention may be administered to human patients suffering from, or at risk of infection by, topical fungal infections caused by among other organisms, species of Candida, Trichophyton, Microsporum or Epidermophyton, or in mucosal infections caused by Candida albicans (e.g. thrush and vaginal candidiasis). The compounds of the invention can also be administered for the treatment or prophylaxis of systemic fungal infections caused by, for example, Candida albicans, Cryptococcus neoformans, Aspergillus flavus, Aspergillus fumigatus, Coccidiodies, Paracoccidioides, Histoplasma or Blastomyces.

In another aspect, the invention provides an antifungal composition for agricultural (including horticultural) use, comprising a compound of the formula (I) together with an agriculturally acceptable diluent or carrier.

The invention further provides a method of treating an animal (including a mammal such as a human), plant or seed having a fungal infection, which comprises treating said animal, plant or seed, or the locus of said plant or seed, with an effective amount of a compound of the formula (I).

The invention also provides a method of treating a fungal infection in a plant or seed which comprises treating the plant or seed with an antifungally effective amount of a fungicidal composition containing a compound of the formula (I) as hereinbefore defined.

Differential screening assays may be used to select for those compounds of the present invention with specificity for non-human CDK enzymes. Compounds which act specifically on the CDK enzymes of eukaryotic pathogens can be used as anti-fungal or anti-parasitic agents. Inhibitors of the Candida CDK kinase, CKSI, can be used in the treatment of candidiasis. Antifungal agents can be used against infections of the type hereinbefore defined, or opportunistic infections that commonly occur in debilitated and immunosuppressed patients such as patients with leukemias and lymphomas, people who are receiving immunosuppressive therapy, and patients with predisposing conditions such as diabetes mellitus or AIDS, as well as for non-immunosuppressed patients.

Assays described in the art can be used to screen for agents which may be useful for inhibiting at least one fungus implicated in mycosis such as candidiasis, aspergillosis, mucormycosis, blastomycosis, geotrichosis, cryptococcosis, chromoblastomycosis, coccidiodomycosis, conidiosporosis, histoplasmosis, maduromycosis, rhinosporidosis, nocaidiosis, para-actinomycosis, penicilliosis, monoliasis, or sporotrichosis. The differential screening assays can be used to identify anti-fungal agents which may have therapeutic value in the treatment of aspergillosis by making use of the CDK genes cloned from yeast such as Aspergillus fumigatus, Aspergillus flavus, Aspergillus niger, Aspergillus nidulans, or Aspergillus terreus, or where the mycotic infection is mucon-nycosis, the CDK assay can be derived from yeast such as Rhizopus althizus, Rhizopus oryzae, Absidia corymbifera, Absidia ramosa, or Mucoipusillus. Sources of other CDK enzymes include the pathogen Pneumocystis carinii.

By way of example, in vitro evaluation of the antifungal activity of the compounds can be performed by determining the minimum inhibitory concentration (M.I.C.) which is the lowest concentration of the test compounds, in a suitable medium, at which growth of the particular microorganism fails to occur. In practice, a series of agar plates, each having the test compound incorporated at a particular concentration is inoculated with a standard culture of, for example, Candida albicans and each plate is then incubated for an appropriate period at 37° C. The plates are then examined for the presence or absence of growth of the fungus and the appropriate M.I.C. value is noted. Alternatively, a turbidity assay in liquid cultures can be performed and a protocol outlining an example of this assay can be found in Example 8.

The in vivo evaluation of the compounds can be carried out at a series of dose levels by intraperitoneal or intravenous injection or by oral administration, to mice that have been inoculated with a fungus, e.g., a strain of Candida albicans or Aspergillus flavus. The activity of the compounds can be assessed by monitoring the growth of the fungal infection in groups of treated and untreated mice (by histology or by retrieving fungi from the infection). The activity may be measured in terms of the dose level at which the compound provides 50% protection against the lethal effect of the infection (PD₅₀).

For human antifungal use, the compounds of the formula (I) can be administered alone or in admixture with a pharmaceutical carrier selected in accordance with the intended route of administration and standard pharmaceutical practice. Thus, for example, they may be administered orally, parenterally, intravenously, intramuscularly or subcutaneously by means of the formulations described above in the section headed “Pharmaceutical Formulations”.

For oral and parenteral administration to human patients, the daily dosage level of the antifungal compounds of the formula (I) can be from 0.01 to 10 mg/kg (in divided doses), depending on inter alia the potency of the compounds when administered by either the oral or parenteral route. Tablets or capsules of the compounds may contain, for example, from 5 mg to 0.5 g of active compound for administration singly or two or more at a time as appropriate. The physician in any event will determine the actual dosage (effective amount) which will be most suitable for an individual patient and it will vary with the age, weight and response of the particular patient.

Alternatively, the antifungal compounds of formula (I) can be administered in the form of a suppository or pessary, or they may be applied topically in the form of a lotion, solution, cream, ointment or dusting powder. For example, they can be incorporated into a cream consisting of an aqueous emulsion of polyethylene glycols or liquid paraffin; or they can be incorporated, at a concentration between 1 and 10%, into an ointment consisting of a white wax or white soft paraffin base together with such stabilizers and preservatives as may be required.

In addition to the therapeutic uses described above, anti-fungal agents developed with such differential screening assays can be used, for example, as preservatives in foodstuff, feed supplement for promoting weight gain in livestock, or in disinfectant formulations for treatment of non-living matter, e.g., for decontaminating hospital equipment and rooms. In similar fashion, side by side comparison of inhibition of a mammalian CDK and an insect CDK, such as the Drosophilia CDK5 gene (Hellmich et al. (1994) FEBS Lett 356:317-21), will permit selection amongst the compounds herein of inhibitors which discriminate between the human/mammalian and insect enzymes. Accordingly, the present invention expressly contemplates the use and formulation of the compounds of the invention in insecticides, such as for use in management of insects like the fruit fly.

In yet another embodiment, certain of the subject CDK inhibitors can be selected on the basis of inhibitory specificity for plant CDK's relative to the mammalian enzyme. For example, a plant CDK can be disposed in a differential screen with one or more of the human enzymes to select those compounds of greatest selectivity for inhibiting the plant enzyme. Thus, the present invention specifically contemplates formulations of the subject CDK inhibitors for agricultural applications, such as in the form of a defoliant or the like.

For agricultural and horticultural purposes the compounds of the invention may be used in the form of a composition formulated as appropriate to the particular use and intended purpose. Thus the compounds may be applied in the form of dusting powders, or granules, seed dressings, aqueous solutions, dispersions or emulsions, dips, sprays, aerosols or smokes. Compositions may also be supplied in the form of dispersible powders, granules or grains, or concentrates for dilution prior to use. Such compositions may contain such conventional carriers, diluents or adjuvants as are known and acceptable in agriculture and horticulture and they can be manufactured in accordance with conventional procedures. The compositions may also incorporate other active ingredients, for example, compounds having herbicidal or insecticidal activity or a further fungicide. The compounds and compositions can be applied in a number of ways, for example they can be applied directly to the plant foliage, stems, branches, seeds or roots or to the soil or other growing medium, and they may be used not only to eradicate disease, but also prophylactically to protect the plants or seeds from attack. By way of example, the compositions may contain from 0.01 to 1 wt. % of the active ingredient. For field use, likely application rates of the active ingredient may be from 50 to 5000 g/hectare.

The invention also contemplates the use of the compounds of the formula (I) in the control of wood decaying fungi and in the treatment of soil where plants grow, paddy fields for seedlings, or water for perfusion. Also contemplated by the invention is the use of the compounds of the formula (I) to protect stored grain and other non-plant loci from fungal infestation.

### EXAMPLES

The invention will now be illustrated, but not limited, by reference to the specific embodiments described in the following examples.

Analytical LC-MS System

In the examples, the compounds prepared were characterised by liquid chromatography and mass spectroscopy using the systems and operating conditions set out below. Where chlorine is present, the mass quoted for the compound is for ³⁵Cl. Several systems were used, as described below, and these were equipped with were set up to run under closely similar operating conditions. The operating conditions used are also described below.

| | |
|---|---|
| HPLC System: | Waters 2795 |
| Mass Spec Detector: | Micromass Platform LC |
| PDA Detector: | Waters 2996 PDA |

Acidic Analytical Conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/mm |
| Column: | Phenomenex Synergi 4 μ MAX-RP 80A, 2.0 × 50 mm |

Basic Analytical Conditions:

| | |
|---|---|
| Eluent A: | H₂O (10 mM NH₄HCO₃ buffer adjusted to pH = 9.5 |
| | with NH₄OH) |
| Eluent B: | CH₃CN |
| Gradient: | 05-95% eluent B over 3.5 minutes |
| Flow: | 0.8 ml/min |
| Column: | Thermo Hypersil-Keystone BetaBasic-18 5 μm 2.1 × 50 mm |
| or | |
| Column: | Phenomenex Luna C18(2) 5 μm 2.0 × 50 mm |

Polar Analytical Conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 0.8 ml/min |
| Column: | Thermo Hypersil-Keystone HyPurity Aquastar, |
| or | 5 μ, 2.1 × 50 mm |
| Column: | Phenomenex Synergi 4 μ MAX-RP 80A, 2.0 × 50 mm or |

Longer Analytical Conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4 μ MAX-RP 80A, 2.0 × 150 mm |

MS Conditions:

| | |
|---|---|
| Capillary voltage: | 3.6 kV |
| Cone voltage: | 30 V |
| Source Temperature: | 120° C. |
| Scan Range: | 165-700 amu |
| Ionisation Mode: | ElectroSpray Positive or |
| | ElectroSpray Negative or |
| | ElectraSpray Positive & Negative |

Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z, Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

Hardware:

Waters Fractionlynx system:

2767 Dual Autosampler/Fraction Collector

2525 preparative pump

CFO (column fluidic organizer) for column selection

RMA (Waters reagent manager) as make up pump

Waters ZQ Mass Spectrometer

Waters 2996 Photo Diode Array detector

Software:

Masslynx 4.0

Columns:

1. Low pH chromatography:

Phenomenex Synergy MAX-RP, 10μ, 150×15 mm

(alternatively used same column type with 100×21.2 mm dimensions).

2. High pH chromatography:

Phenomenex Luna C18 (2), 10μ, 100×21.2 mm

(alternatively used Thermo Hypersil Keystone BetaBasic C18, 5μ, 100×21.2 mm)

Eluents:

1. Low pH chromatography:

Solvent A: H₂0+0.1% Formic Acid, pH 1.5

Solvent B: CH₃CN+0.1% Formic Acid

2. High pH chromatography:

Solvent A: H₂0+10 mM NH₄HCO₃+NH₄OH, pH 9.5

Solvent B: CH₃CN

3. Make up solvent:

MeOH+0.1% Formic Acid (for both chromatography type)

Methods:

According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min

Gradient: Generally all gradients had an initial 0.4 min step with 95% A+5% B. Then according to analytical trace a 3.6 min gradient was chosen in order to achieve good separation (e.g. from 5% to 50% B for early retaining compounds; from 35% to 80% B for middle retaining compounds and so on)

Wash: 1 minute wash step was performed at the end of the gradient

Re-equilibration: 2.1 minute re-equilibration step was ran to prepare the system for the next run

Make Up flow rate: 1 ml/min

Solvent:

All compounds were usually dissolved in 100% MeOH or 100% DMSO

MS Running Conditions:

| | |
|---|---|
| Capillary voltage: | 3.2 kV |
| Cone voltage: | 25 V |
| Source Temperature: | 120° C. |
| Multiplier: | 500 V |
| Scan Range: | 125-800 amu |
| Ionisation Mode: | ElectroSpray Positive |

From the information provided someone skilled in the art could purify the compounds described herein by preparative LC-MS.

The starting materials for each of the Examples are commercially available unless otherwise specified.

### Example 1

### Synthesis of 2,6-difluoro-N-[3-(4-oxo-4,5-dihydro-3H-imidazo[4,5-c]pyridin-2-yl)-1H-pyrazol-4-yl]-benzamide

### 1A. 4-Nitro-1H-pyrazole-3-carboxylic acid ethyl ester

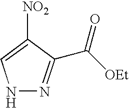

Thionyl chloride (2.90 ml, 39.8 mmol) was slowly added to a mixture of 4-nitro-3-pyrazolecarboxylic acid (5.68 g, 36.2 mmol) in EtOH (100 ml) at ambient temperature and the mixture stirred for 48 h. The mixture was reduced in vacuo and dried through azeotrope with toluene to afford 4-nitro-1H-pyrazole-3-carboxylic acid ethyl ester as a white solid (6.42 g, 96%). (¹H NMR (400 MHz, DMSO-d₆) δ 14.4 (s, 1H), 9.0 (s, 1H), 4.4 (q, 2H), 1.3 (t, 3H)).

### 1B. Synthesis of 4-Amino-1H-pyrazole-3-carboxylic acid ethyl ester

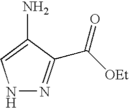

A mixture of 4-nitro-1H-pyrazole-3-carboxylic acid ethyl ester (6.40 g, 34.6 mmol) and 10% Pd/C (650 mg) in EtOH (150 ml) was stirred under an atmosphere of hydrogen for 20 h. The mixture was filtered through a plug of Celite, reduced in vacuo and dried through azeotrope with toluene to afford 4-amino-1H-pyrazole-3-carboxylic acid ethyl ester as a pink solid (5.28 g, 98%). (¹H NMR (400 MHz, DMSO-d₆) δ 12.7 (s, 1H), 7.1 (s, 1H), 4.8 (s, 2H), 4.3 (q, 2H), 1.3 (t, 3H)).

### 1C. Synthesis of 4-(2,6-Difluoro-benzoylamino)-1H-pyrazole-3-carboxylic acid ethyl ester

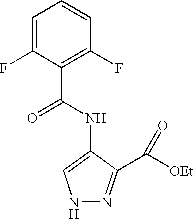

A mixture of 2,6-difluorobenzoic acid (6.32 g, 40.0 mmol), 4-amino-1H-pyrazole-3-carboxylic acid ethyl ester (5.96 g, 38.4 mmol), EDC (8.83 g, 46.1 mmol) and HOBt (6.23 g, 46.1 mmol) in DMF (100 ml) was stirred at ambient temperature for 6 h. The mixture was reduced in vacuo, water added and the solid formed collected by filtration and air-dried to give 4-(2,6-difluoro-benzoylamino)-1H-pyrazole-3-carboxylic acid ethyl ester as the major component of a mixture (15.3 g). (LC/MS: Rₜ 3.11, [M+H]⁺ 295.99).

### 1D. Synthesis of 4-(2,6-Difluoro-benzoylamino)-1H-pyrazole-3-carboxylic acid

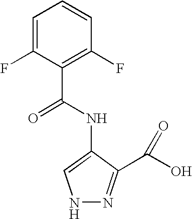

A mixture of 4-(2,6-difluoro-benzoylamino)-1H-pyrazole-3-carboxylic acid ethyl ester (10.2 g) in 2 M aqueous NaOH/MeOH (1:1, 250 ml) was stirred at ambient temperature for 14 h. Volatile materials were removed in vacuo, water (300 ml) added and the mixture taken to pH 5 using 1M aqueous HCl. The resultant precipitate was collected by filtration and dried through azeotrope with toluene to afford 4-(2,6-difluoro-benzoylamino)-1H-pyrazole-3-carboxylic acid as a pink solid (5.70 g). (LC/MS: Rₜ 2.33, [M+H]⁺ 267.96).

### 1E. Synthesis of 3,4-diamino-1H-pyridin-2-one

4-Chloro-3-nitro-2-pyridone (1 g, 0.57 mmol) in 2 M methanolic ammonia was heated in the microwave (50 W) at 110° C. until the reaction was complete. Reaction evaporated to dryness, to give 3-amino-4-nitro-1H-pyridin-2-one as a brown solid (0.4 g). (LC/MS: Rₜ 0.36).

3-Amino-4-nitro-1H-pyridin-2-one (0.2 g, 1.29 mmol) taken up in hot DMF (10 ml) and hydrogenated using 10% Pd/C. The reaction mixture was filtered through Celite and the filtrate evaporated to give a brown oil, which solidified affording the title compound. (LC/MS: Rₜ 0.36, [M+H]⁺ 126).

### 1F. Synthesis of 2,6-difluoro-N-[3-(4-oxo-4,5-dihydro-3H-imidazo[4,5-c]pyridin-2-yl)-1H-pyrazol-4-yl]-benzamide

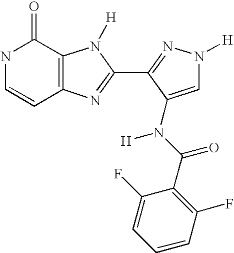

A mixture of 4-(2,6-difluorobenzoylamino)-1H-pyrazole-3-carboxylic acid (100 mg, 0.37 mmol), 3,4-diamino-1H-pyridin-2-one (54 mg, 0.37 mmol), EDC (72 mg, 0.40 mmol) and HOBt (57.3 mg, 0.40 mmol) in DMF (2 ml) was stirred at ambient temperature for 24 h and then partitioned between EtOAc and saturated aqueous sodium bicarbonate. The solid amide intermediate crystallised, was filtered and washed with EtOAc. The intermediate was dissolved in AcOH (2 ml) and this mixture heated in the microwave (150 W) at 160° C. until reaction was complete. The reaction mixture was allowed to cool, a solid crystallised out, which was filtered and then washed with ether and dried to give the required product (20 mg). (LC/MS: Rₜ 2.14, [M+H]⁺ 357).

### Example 2

### Synthesis of 5-Methyl-4-morpholin-4-ylmethyl-furan-2-carboxylic acid [3-(5-benzyl-4-oxo-4,5 dihydro-1H-imidazo[4,5-d]pyridazin-2-yl)-1H-pyrazol-4-yl]-amide

### 2A. Synthesis of 4-[(5-Methyl-4-morpholin-4-ylmethyl-furan-2-carbonyl)-amino]-1H-pyrazole-3-carboxylic acid

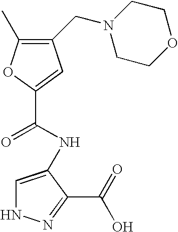

4-[(5-Methyl-4-morpholin-4-ylmethyl-furan-2-carbonyl)-amino]-1H-pyrazole-3-carboxylic acid was prepared as outlined above using 5-methyl-4-(morpholin-4-ylmethyl)-2-furoic acid (commercially available from Bionet) in the place of 2,6-difluorobenzoic acid.

### 2B. 5-Methyl-4-morpholin-4-ylmethyl-furan-2-carboxylic acid [3-(5-benzyl-4-oxo-4,5 dihydro-1H-imidazo[4,5-d]pyridazin-2-yl)-1H-pyrazol-4-yl]-amide

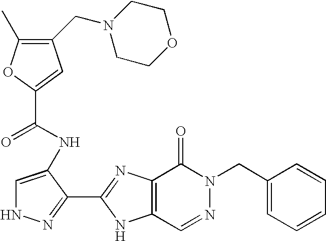

3,4-diamino-2-benzyl-3 (2H)-pyridazinone (0.10 g, 0.46 mmol) (commercially available from SPECS), 4-[(5-Methyl-4-morpholin-4-ylmethyl-furan-2-carbonyl)-amino]-1H-pyrazole-3-carboxylic acid (0.080 g, 0.30 mmol), EDC (0.121 g, 0.55 mmol) and HOAt (0.075 g, 0.55 mmol) were dissolved in DMF (5 ml) and heated at 80° C. for 1 hour then stirred at ambient temperature for 18 hours. The reaction mixture was reduced in vacuo and the residue was partitioned between ethyl acetate (50 ml) and saturated aqueous sodium bicarbonate solution (50 ml). The organic layer was washed with brine, dried (MgSO₄) and reduced in vacuo to give the intermediate amide. Acetic acid (3 ml) was added to the crude amide and the mixture was heated at 120° C. for 3 hours, solid precipitated on cooling, solid filtered off then washed with ethyl acetate followed by ether to give the desired product 5-Methyl-4-morpholin-4-ylmethyl-furan-2-carboxylic acid [3-(5-benzyl-4-oxo-4,5-dihydro-1H-imidazo[4,5-d]pyridazin-2-yl)-1H-pyrazol-4-yl]-amide (0.030 g) as a white solid. (LC/MS: Rₜ 2.14, [M+H]⁺ 515).

### Example 3

### Synthesis of 5-Methyl-4-morpholin-4-ylmethyl-furan-2-carboxylic acid [3-(5-methyl-4-oxo-4,5-dihydro-1H-imidazo[4,5-d]pyridazin-2-yl)-1H-pyrazol-4-yl]-amide

### 3A. 4-Hydroxy-2-methyl-5-nitro-2H-pyridazin-3-one

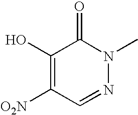

To a solution of 4,5-dichloro-2-methylpyridazin-3-one (1 g, 5.58 mmol) in DMF (20 ml) was added sodium nitrite (1.54 g, 22 mmol) and the mixture heated at 90° C. for 24 hours. The reaction was reduced in vacuo, the residue dissolved in warm 6M HCl (4 ml) and allowed to cool to ambient temperature. The solid was filtered off and triturated with ether to yield 4-Hydroxy-2-methyl-5-nitro-2H-pyridazin-3-one (0.910 g) (LC/MS (polar method): Rₜ 1.79, [M−H]⁻ 170)

### 3B. 4-Amino-2-methyl-5-nitro-2H-pyridazin-3-one

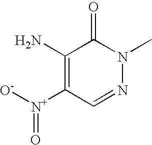

A suspension of 4-hydroxy-2-methyl-5-nitro-2H-pyridazin-3-one (0.6 g, 3.5 mmol) in saturated methanolic ammonia (15 ml) was heated in a sealed tube to 130° C. for 36 hours then reduced in vacuo to yield 4-Amino-2-methyl-5-nitro-2H-pyridazin-3-one (0.55 g) (LC/MS (polar method): Rₜ 2.00, no molecular ion).

### 3C. 4,5-Diamino-2-methyl-2H-pyridazin-3-one

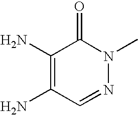

A mixture of 4-amino-2-methyl-5-nitro-2H-pyridazin-3-one (0.2 g, 1.17 mmol) and platinum oxide (50 mg) in EtOH/EtOAc [1:1] (15 ml) was shaken under an atmosphere of hydrogen for 3 hours. The reaction mixture was filtered through GF/A paper and reduced in vacuo to yield 4,5-Diamino-2-methyl-2H-pyridazin-3-one (0.164 g) (LC/MS (polar method): Rₜ 0.38, [M+H]⁺ 141)

### 3D. 4-[(5-Methyl-4-morpholin-4-ylmethyl-furan-2-carbonyl)-amino]-1H-pyrazole-3-carboxylic acid methyl ester

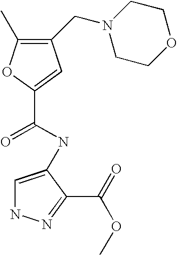

A mixture of 5-methyl-4-(morpholin-4-ylmethyl)-2-furoic acid (0.37 g, 1.64 mmol), 4-amino-1H-pyrazole-3-carboxylic acid methyl ester (0.23 g, 1.64 mmol), EDAC (0.347 g, 1.8 mmol) and HOBt (0.244 g, 1.8 mmol) in DMF (5 ml) was stirred at ambient temperature for 16 h. The mixture was partitioned between EtOAc and saturated bicarbonate, organic portion washed with brine, reduced in vacuo, to yield 4-[(5-Methyl-4-morpholin-4-ylmethyl-furan-2-carbonyl)-amino]-1H-pyrazole-3-carboxylic acid methyl ester as a cream solid (0.37 g). (LC/MS (acidic method): Rₜ 1.59, [M+H]⁺ 349).

### 3E. 4-[(5-Methyl-4-morpholin-4-ylmethyl-furan-2-carbonyl)-amino]-1H-pyrazole-3-carboxylic acid

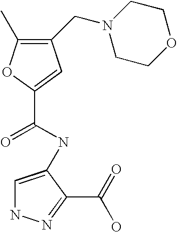

A mixture of 4-[(5-Methyl-4-morpholin-4-ylmethyl-furan-2-carbonyl)-amino]-1H-pyrazole-3-carboxylic acid methyl ester (0.37 g) in 2N aqueous NaOH (2.5 ml) and MeOH (10 ml) was stirred at 60 C for 2 hours. Reaction mixture reduced in vacuo, then acidified and the resultant precipitate filtered, washed with water and dried to yield 4-[(5-Methyl-4-morpholin-4-ylmethyl-furan-2-carbonyl)-amino]-1H-pyrazole-3-carboxylic acid as a cream solid (5.70 g). (LC/MS (acidic method): Rₜ 1.16, [M+H]⁺ 335).

### 3F. 5-Methyl-4-morpholin-4-ylmethyl-furan-2-carboxylic acid [3-(5-methyl-4-oxo-4,5-dihydro-1H-imidazo[4,5-d]pyridazin-2-yl)-1H-pyrazol-4-yl]-amide

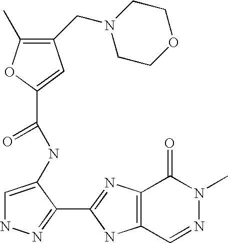

A mixture of 4-[(5-methyl-4-morpholin-4-ylmethyl-furan-2-carbonyl)-amino]-1H-pyrazole-3-carboxylic acid (0.195 g, 0.58 mmol), 4,5-Diamino-2-methyl-2H-pyridazin-3-one (0.82 g, 0.58 mmol), EDC (0.153 g, 0.69 mmol) and HOAt (0.095 g, 0.69 mmol) in DMF (5 ml) was stirred at 80° C. for 3 hour then at ambient temperature for 48 hours and then reduced in vacuo. The residue was partitioned between EtOAc and saturated bicarbonate, the organic portion was washed with brine and dried (MgSO₄), filtered and reduced in vacuo. The crude amide intermediate (0.1 g), (LC/MS (acidic method): Rₜ 1.6, [M+H]⁺ 457) was dissolved in AcOH (3 ml) then heated for 40 mins at 160° C. (150 W) in a CEM discover microwave. The reaction mixture was reduced in vacuo, residue purified by preparative LC to yield (5-Methyl-4-morpholin-4-ylmethyl-furan-2-carboxylic acid [3-(5-methyl-4-oxo-4,5-dihydro-1H-imidazo[4,5-d]pyridazin-2-yl)-1H-pyrazol-4-yl]-amide (9 mg). (LC/MS (acidic method): Rₜ 1.83, [M+H]⁺ 439)

### Example 4

### Synthesis of 2,6-difluoro-N-[3-(4,5,6,7-tetrahydro-1H-imidazol[4,5-c]pyridin-2-yl)-1H-pyrazol-4-yl]-benzamide

### 4A. Synthesis of 4-Nitro-1H-pyrazole-3-carboxylic acid methyl ester

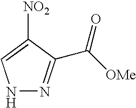

Thionyl chloride (3.8 ml, 52.5 mmol) was added cautiously to a stirred, ice-cold mixture of 4-nitropyrazole-3-carboxylic acid (7.5 g, 47.7 mmol) in MeOH (150 ml), the mixture stirred at ambient temperature for 1 hour then heated at reflux for 3 hours. The reaction mixture was cooled, evaporated in vacuo then azeotroped with toluene to give 4-nitro-1H-pyrazole-3-carboxylic acid ethyl ester (8.8 g).

### 4B. Synthesis of 4-nitro-1-(tetrahydro-pyran-2-yl)-1H-pyrazole-3-carboxylic acid methyl ester

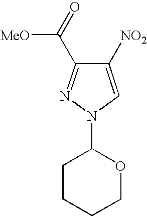

A suspension of 4-nitro-1H-pyrazole-3-carboxylic acid methyl ester (5 g, 29.24 mmol) and p-toluene sulphonic acid (555 mg, 2.92 mmol) in chloroform (100 ml) at 0° C. was treated with 3,4-dihydropyran (4 ml, 43.8 mmol) dropwise. The reaction mixture was allowed to warm to ambient temperature, and then stirred for a further 2 hours. The reaction mixture was diluted with Et₂O, washed sequentially with saturated NaHCO₃ solution and brine. The organic portion was dried (MgSO₄), filtered and evaporated in vacuo. The residue was purified by flash chromatography [silica, EtOAc/Petrol (1:2)] to give 4-nitro-1-(tetrahydro-pyran-2-yl)-1H-pyrazole-3-carboxylic acid methyl ester (7.1 g, 95%) as a colourless oil. (LC/MS: Rₜ 2.86, [M+H]⁺ 256.00).

### 4C. Synthesis of 4-amino-1-(tetrahydro-pyran-2-yl)-1H-pyrazole-3-carboxylic acid methyl ester

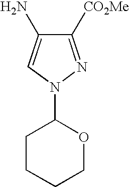

To a stirred solution of 4-nitro-1-(tetrahydro-pyran-2-yl)-1H-pyrazole-3-carboxylic acid methyl ester (16.0 g, 62.75 mmoles, example 14B) and ammonium formate (39.6 g, 627.45 mmoles) and in ethanol (200 ml) and water (20 ml) under nitrogen was added palladium on carbon (10%, 0.8 g). The reaction mixture was heated at 50° C. for 2 hours. The suspension was filtered through celite, and the filtrate was partitioned between EtOAc and water. The organic portion was dried (MgSO₄) to give 4-amino-1-(tetrahydro-pyran-2-yl)-1H-pyrazole-3-carboxylic acid methyl ester as a yellow oil (12.5 g, 89%). (LC/MS: Rₜ 1.84, [M+H]⁺ 226.06).

### 4D. Synthesis of 4-(2,6-difluoro-benzoylamino)1-(tetrahydro-pyran-2-yl-1H-pyrazole-3-carboxylic acid methyl ester

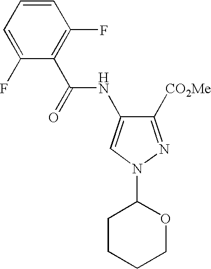

A solution of 4-amino-1-(tetrahydro-pyran-2-yl)-1H-pyrazole-3-carboxylic acid methyl ester (12.5 g, 55.56 mmoles), EDC (18.78 g, 97.96 mmoles), HOBt (13.00 g, 96.30 mmoles) and 2,6-difluorobenzoic acid (12.8 g, 81.01 mmoles) in dichloromethane was stirred at ambient temperature for 24 hours, and then partitioned between EtOAc and NaOH solution (2N). The organic portion was dried (MgSO₄), filtered and evaporated in vacuo. The residue was purified [Biotage SP4, 3×40M, flow rate 40 ml/min, gradient 3:7 EtOAc/Petrol to 2:1 EtOAc/Petrol] to give 4-(2,6-difluoro-benzoylamino) 1-(tetrahydro-pyran-2-yl-1H-pyrazole-3-carboxylic acid methyl ester as a white solid (11.3 g, 56%). (LC/MS: Rₜ 3.10, [M+H]⁺ 366.19).

### 4E. Synthesis of 2,6-difluoro-N-[3-hydroxymethyl-1-(tetrahydro-pyran-2-yl)-1H-pyrazol-4-yl]-benzamide

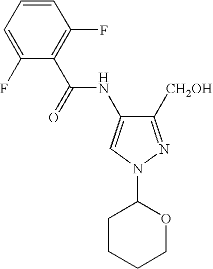

A stirred solution of 4-(2,6-difluoro-benzoylamino)1-(tetrahydro-pyran-2-yl-1H-pyrazole-3-carboxylic acid methyl ester (11.3 g, 30.96 mmoles) in THF (500 ml) under nitrogen at −78° C. was treated dropwise with a solution of diisobutylaluminium hydride in THF (310 ml, 1M). The reaction mixture was stirred at −78° C. for 1 hour and then warmed to 0° C. in an ice-water bath. A saturated aqueous solution of sodium sulphate (300 ml) was added to the reaction mixture. The suspension was filtered through celite. The filtrate was partitioned between EtOAc and brine. The organic portion was dried (MgSO₄), filtered and evaporated in vacuo to give 2,6-difluoro-N-[3-hydroxymethyl-1-(tetrahydro-pyran-2-yl)-1H-pyrazol-4-yl]-benzamide as a white solid (10.14 g, 97%). (LC/MS: Rₜ 2.34, [M+H]⁺ 338.03).

### 4F. Synthesis of 2,6-difluoro-N-[3-formyl-1-(tetrahydro-pyran-2-yl)-1H-pyrazol-4-yl]-benzamide

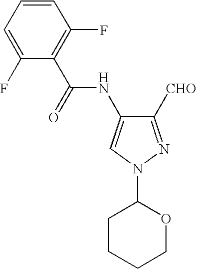

To a stirred solution of 2,6-difluoro-N-[3-hydroxymethyl-1-(tetrahydro-pyran-2-yl)-1H-pyrazol-4-yl]-benzamide (10.14 g, 30.10 mmoles) in acetone (200 ml) was added MnO₂ (52.33 g, 602 mmoles). The resultant black suspension was stirred at ambient temperature for 48 hours. The reaction mixture was filtered through celite, and the fitrate evaporated in vacuo. The residue was purified by flash column chromatography (Biotage SP4, 40M, flow rate 30 ml/min, gradient 1:3 EtOAc/Petrol to 3:2 EtOAc/Petrol) to give 2,6-difluoro-N-[3-formyl-1-(tetrahydro-pyran-2-yl)-1H-pyrazol-4-yl]-benzamide as a creamy solid (7.8 g, 77%). (LC/MS: Rₜ 3.03, [M+H]⁺ 336.03).

### 4G. Synthesis of 3-hydroxyimino-4-oxo-piperidine-1-carboxylic acid benzyl ester

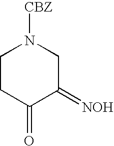

To a stirred solution of 4-oxo-piperidine-1-carboxylic acid benzyl ester (6.0 g, 25.75 mmoles) in dichoromethane (10 ml) under nitrogen at −20° C. was added dropwise TMSCl (3.25 ml, 25.75 mmoles) and then isoamyl nitrite (3.46 ml, 25.75 mmoles). The reaction mixture was then stirred at ambient temperature for 30 minutes. The reaction mixture was purified [Biotage SP4, 40M, flow rate 40 ml/min, 3:2 EtOAc/Petrol to 4:1 EtOAc/Petrol] to give 3-hydroxyimino-4-oxo-piperidine-1-carboxylic acid benzyl ester as a yellow oil (1.6 g, 24%). (LC/MS: Rₜ 2.50, [M+H]⁺ 263.00).

### 4H. Synthesis of 2-[4-2,6-difluoro-benzoylamino)-1H-pyrazol-3-yl]-1,4,6,7-tetrahydro-imidazol[4,5-c]pyridine-5-carboxylic acid benzyl ester

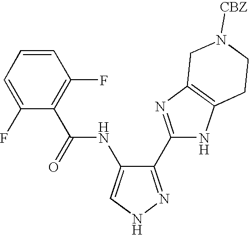

A solution of 3-hydroxyimino-4-oxo-piperidine-1-carboxylic acid benzyl ester (524 mg, 2 mmoles), ammonium acetate (462 mg, 6 mmoles) and 2,6-difluoro-N-[3-formyl-1-(tetrahydro-pyran-2-yl)-1H-pyrazol-4-yl]-benzamide (335 mg, 1 mmole) in acetic acid (2 ml) was heated at 150° C. (80 W) in a CEM discover microwave synthesiser for 10 minutes. The reaction mixture was partitioned between EtOAc and a solution of NaOH (2N). The organic portion was dried (MgSO₄), filtered and evaporated in vacuo. The residue was purified [Biotage SP4, 25S, flow rate 20 ml/min, gradient 3:1 EtOAc/Petrol to EtOAc] to give 2-[4-2,6-difluoro-benzoylamino)-1H-pyrazol-3-yl]-1,4,6,7-tetrahydro-imidazol[4,5-c]pyridine-5-carboxylic acid benzyl ester as a pale yellow solid (150 mg, 31%). (LC/MS: Rₜ 2.65, [M+H]⁺ 479.01).

### 4I. Synthesis of 2,6-difluoro-N-[3-(4,5,6,7-tetrahydro-1H-imidazol[4,5-c]pyridin-2-yl)-1H-pyrazol-4-yl]-benzamide

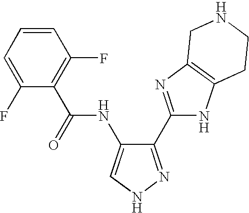

To a solution of 2-[4-2,6-difluoro-benzoylamino)-1H-pyrazol-3-yl]-1,4,6,7-tetrahydro-imidazol[4,5-c]pyridine-5-carboxylic acid benzyl ester (150 mg, 0.304 mmoles) in ethanol (10 ml) was added under nitrogen water (1 ml), ammonium formate (191 mg, 3.036 mmoles) and palladium on carbon (10%, 8 mg). The suspension was heated at 70° C. for 1 hour. The suspension was filtered through celite and the filtrate partitioned between water and EtOAc. The organic portion was dried (MgSO₄), filtered and the solvent was removed in vacuo. The residue was triturated with ether and filtered to give 6-difluoro-N-[3-(4,5,6,7-tetrahydro-1H-imidazol[4,5-c]pyridin-2-yl)-1H-pyrazol-4-yl]-benzamide as a pale yellow solid (26 mg, 25%). (LC/MS: Rₜ 2.02, [M+H]⁺ 345.02).

### Example 5

### Synthesis of 2,6-difluoro-N-[3-(1,4,6,7-tetrahydro-thiopyrano[3,4-d]imidazol-2-yl)-1H-pyrazol-4-yl]-benzamide

### 5A. Synthesis of dihydro-thiopyran-3,4-dione-3-oxime

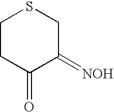

To a stirred solution of tetrahydrothiopyran-4-one (2.5 g, 21.55 mmoles) in dichloromethane (5 ml) under nitrogen at −20° C. was added dropwise TMSCl (2.7 ml, 21.55 moles) and then isoamyl nitrite (2.9 ml, 21.55 moles). The reaction mixture was stirred at −20° C. for 20 minutes. The reaction mixture was purified by flash chromatography [Biotage SP4, 40M, flow rate 40 ml/min, gradient 1:3 EtOAc/Petrol to 4:1 EtOAc/Petrol] to give a dihydro-thiopyran-3,4-dione-3-oxime as a yellow-brown oil (0.17 g, 5%). (LC/MS: Rₜ 1.64, [M+H]⁺ 146.01).

### 5B. Synthesis of 2,6-difluoro-N-[3-(1,4,6,7-tetrahydro-thiopyrano[3,4-d]imidazol-2-yl)-1H-pyrazol-4-yl]-benzamide

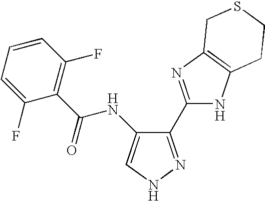

A solution of dihydro-thiopyran-3,4-dione-3-oxime (150 mg, 1.03 mmoles), ammonium acetate (476 mg, 6.18 mmoles) and 2,6-difluoro-N-[3-formyl-1-(tetrahydro-pyran-2-yl)-1H-pyrazol-4-yl]-benzamide (312 mg, 0.93 mmoles) in acetic acid (2 ml) was heated at 150° C. (80 W) in a CEM discover microwave synthesizer for 20 minutes. The reaction mixture was partitioned between EtOAc and a solution of sodium hydroxide solution (2N). The organic portion was dried (MgSO₄), filtered and evaporated in vacuo. The residue was purified firstly by flash chromatography [Biotage SP4, 25S, flow rate 25 ml/min, gradient 1:1 EtOAc/Petrol to EtOAc] and then secondly by trituration with diethyl ether to give 2,6-difluoro-N-[3-(1,4,6,7-tetrahydro-thiopyrano[3,4-d]imidazol-2-yl)-1H-pyrazol-4-yl]-benzamide as a light brown solid (30 mg, 9%). (LC/MS: Rₜ 1.97, [M+H]⁺ 362.05).

### Example 6

### Synthesis of N-[3-(5-ethyl-4,5,6,7-tetrahydro-1H-imidazo[4,5-c]pyridine-2-yl)-1H-pyrazol-4-yl]-2,6-difluoro-benzamide

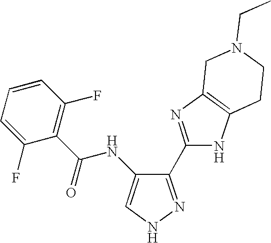

To a stirred solution of 2,6-difluoro-N-[3-(4,5,6,7-tetrahydro-1H-imidazol[4,5-c]pyridine-2-yl)-1H-pyrazol-4-yl]-benzamide (Example 4I) (50 mg, 0.15 mmoles) in 1,2-dichloroethane was added acetaldehyde (8 μl, 0.15 mmoles), acetic acid (10 μl, 0.18 mmoles) and sodium triacetoxyborohydride (38 mg, 0.18 mmoles). The reaction mixture was stirred at ambient temperature for 3 hours. Further acetaldyde (8 μl, 0.15 mmoles), acetic acid (10 μl, 0.18 mmoles) and sodium triacetoxyborohydride (38 mg, 0.18 mmoles) were added to the reaction mixture, and then the reaction mixture was stirred at ambient temperature for a further 24 hours. The reaction mixture was partitioned between ethyl acetate and a saturated solution of sodium bicarbonate solution. The organic portion was dried (MgSO₄), filtered and evaporated in vacuo. The residue was purified by trituration with diethyl ether and then filtered to give N-[3-(5-ethyl-4,5,6,7-tetrahydro-1H-imidazo[4,5-c]pyridine-2-yl)-1H-pyrazol-4-yl]-2,6-difluoro-benzamide as an off-white solid (10 mg, 18%). (LC/MS: Rₜ 1.93, [M+H]⁺ 373.20).

### Example 7

### Synthesis of 2,6-difluoro-N-{3-[5-(2-fluoro-ethyl)-4,5,6,7-tetrahydro-1H-imidazol[4,5-c]pyridine-2-yl]-1H-pyrazol-4-yl}-benzamide

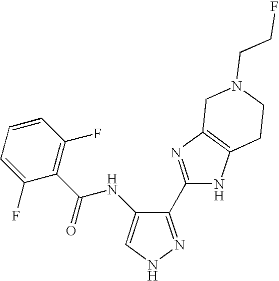

A solution of 2,6-difluoro-N-[3-(4,5,6,7-tetrahydro-1H-imidazol[4,5-c]pyridine-2-yl)-1H-pyrazol-4-yl]-benzamide (Example 4I) (50 mg, 0.15 mmoles), triethylamine (25 μl, 0.18 mmoles) and 1-bromo-2-fluoroethane (11 μl, 0.15 mmoles) in DMF (2 ml) was stirred at ambient temperature for 24 hours. Further triethylamine (100 μl, 0.72 mmoles) and 1-bromo-2-fluoroethane (100 μl, 1.34 mmoles) were added and the resultant solution stirred at ambient temperature for a further 24 hours. The reaction mixture was diluted with ethyl acetate and then washed with water (×2). The organic portion was dried (MgSO₄), filtered and evaporated in vacuo. The residue was purified by trituration with diethyl ether and filtered to give 2,6-difluoro-N-{3-[5-(2-fluoro-ethyl)-4,5,6,7-tetrahydro-1H-imidazol[4,5-c]pyridine-2-yl]-1H-pyrazol-4-yl}-benzamide as a pale yellow solid (10 mg, 17%). (LC/MS: Rt 2.01, [M+H]⁺ 391.19).

### Example 8

### Synthesis of 4,5-Diamino-2-(2-morpholin-4-yl-ethyl)-2H-pyridazin-3-one 8A. 4,5-Dichloro-2-(2-morpholin-4-yl-ethyl)-2H-pyridazin-3-one hydrochloride

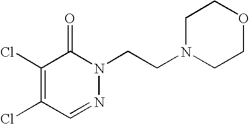

To a suspension of 4,5 dichloro-3-(2H) pyridazinone [ex Aldrich] (4.13 g, 25 mmol) in CH₂Cl₂ was added sodium ethoxide (1.7 g, 25 mmol) followed by N-(2-chloroethyl)morpholine hydrochloride (7.4 g, 39 mmol) in a mixture of sodium ethoxide (2.72 g, 39 mmol) in ethanol (30 ml) and the mixture heated at 80° C. for 1 hour. The reaction was filtered, the filtrate evaporated in vacuo and the residue triturated with ether and filtered again. The filtrate was evaporated in vacuo, then treated with HCl in ether, solid precipitated filtered off then triturated with hot ethanol then washed with ether to yield 4,5-dichloro-2-(2-morpholin-4-ethyl)-2H-pyridazin-3-one (0.93 g) (LC/MS (acidic method): Rₜ 0.39, [M+H]⁺ 278)

### 8B. 4-Hydroxy-2-(2-morpholin-4-yl-ethyl)-5-nitro-2H-pyridazin-3-one

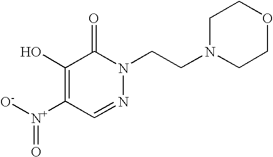

To a solution of 4,5-dichloro-2-(2-morpholin-4-ethyl)-2H-pyridazin-3-one (0.93 g, 3.34 mmol) in DMF (15 ml) added sodium nitrite (0.9 g, 13 mmol) in water (4 ml) was heated at 80° C. for 24 hours then reduced in vacuo, residue dissolved in 4 ml of 6M HCl allowed to cool, solid filtered off and washed with ether to yield 4-hydroxy-2-(2-morpholin-4-yl-ethyl)-5-nitro-2H-pyridazin-3-one (0.55 g) (LC/MS (acidic method): Rₜ 0.40, [M+H]⁺ 271).

### 8C. 4-Amino-2-(2-morpholin-4-yl-ethyl)-5-nitro-2H-pyridazin-3-one

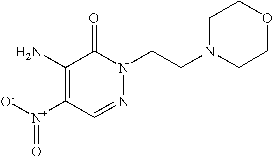

A mixture of 4-hydroxy-2-(2-morpholin-4-yl-ethyl)-5-nitro-2H-pyridazin-3-one (0.4 g, 1.48 mmol) split into four Reacti-vials and treated with freshly prepared methanolic ammonia then heated at 130° C. for 24 hours. Contents of the vials combined and reduced in vacuo, to yield 4-amino-2-(2-morpholin-4-yl-ethyl)-5-nitro-2H-pyridazine-3-one (0.4 g) (LC/MS (acidic method): Rₜ 0.35, [M+H]⁺ 270)

### 8D. 4,5-Diamino-2-(2-morpholin-4-yl-ethyl)-2H-pyridazin-3-one hydrochloride

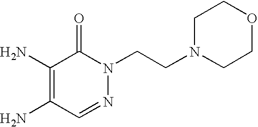

A mixture of 4-amino-2-(2-morpholin-4-yl-ethyl)-5-nitro-2H-pyridazine-3-one (0.2 g, 0.74 mmol) and platinum oxide (30 mg) in EtOH/EtOAc [1:1] (15 ml) was shaken under an atmosphere of hydrogen for 4 hours. The reaction mixture was filtered into a solution of HCl/ether and reduced in vacuo to yield 4,5-diamino-2-(2-morpholin-4-yl-ethyl)-2H-pyridazin-3-one hydrochloride (0.19 g) (LC/MS (acidic method): Rₜ 0.33, [M+H]⁺ 240)

### 8E. S-(−)-4-[(Tetrahydro-furan-2-carbonyl)-amino]-1H-pyrazole-3-carboxylic acid methyl ester

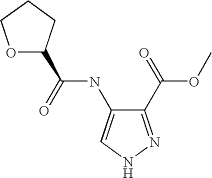

A mixture of 4-amino-1H-pyrazole-3-carboxylic acid methyl ester (1 g, 0.7 mmol), (S)-(−)-2-tetrahydrofuric acid [ex Aldrich] (0.82 g, 0.7 mmol), EDC (1.7 g, 0.77 mmol) and HOBt (0.96 g, 0.77 mmol) in DMF (30 ml) was stirred at ambient temperature for 24 hours and then treated with saturated aqueous sodium bicarbonate. The solid amide product crystallized, was filtered and washed with ether to yield S-(−)-4-[(tetrahydro-furan-2-carbonyl)-amino]-1H-pyrazole-3-carboxylic acid methyl ester (1.44 g). (LC/MS (acidic method): Rₜ 1.96, [M+H]⁺ 240)

### 8F. S-(−)-4-[(Tetrahydro-furan-2-carbonyl)-amino]-1H-pyrazole-3-carboxylic acid

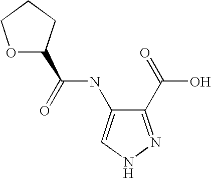

S-(−)-4-[(tetrahydro-furan-2-carbonyl)-amino]-1H-pyrazole-3-carboxylic acid methyl ester (1.44 g, 6.03 mmol) suspended in MeOH (20 ml) treated with NaOH (0.964 g, 24.12 mmol) in water (1 ml) stirred at ambient temperature for 24 hours. The reaction mixture was reduced in vacuo to remove methanol then diluted water and made acidic using HCl and extracted with EtOAc. Organics dried, filtered and reduced in vacuo then triturated with ether followed by EtOAc/ether [1:9] to yield S-(−)-4-[(tetrahydro-furan-2-carbonyl)-amino]-1H-pyrazole-3-carboxylic acid (0.39 g) (LC/MS (acidic method): Rₜ 1.69, [M+H]⁺ 226)

### 8G. S-(−)-Tetrahydro-furan-2-carboxylic acid {3-[5-(2-morpholin-4-yl-ethyl)-4,5-dihydro-1H-imidazo[4,5-d]pyridazin-2-yl]-1H-pyrazol-4-yl}-amide

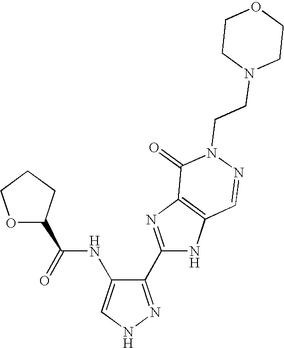

A mixture of S-(−)-4-[(tetrahydro-furan-2-carbonyl)-amino]-1H-pyrazole-3-carboxylic acid (86 mg, 0.382 mmol), 4,5-diamino-2-(2-morpholin-4-yl-ethyl)-2H-pyridazin-3-one hydrochloride (118 mg, 0.382 mmol), EDC (57 mg, 0.42 mmol), HOAt (57 mg, 0.42 mmol) and Et₃N (0.16 ml, 1.14 mmol) dissolved in DMF (2 ml) heated at 80° C. for 4 hours then at ambient overnight then reduced in vacuo. The residue was partitioned between EtOAc and saturated bicarbonate solution; the organic portion was washed with brine and dried (MgSO₄), filtered and reduced in vacuo. The crude amide intermediate (0.117 g) (LC/MS (acidic method): Rₜ 2.04, [M+H]⁺ 448) was dissolved in AcOH (3 ml) then heated for 30 mins at 160° C. (150 W) in a CEM discover microwave. The reaction mixture was reduced in vacuo, residue purified by preparative HPLC followed by trituration with methanol to yield S-(−)-tetrahydro-furan-2-carboxylic acid {3-[5-(2-morpholin-4-yl-ethyl)-4,5-dihydro-1H-imidazo[4,5-d]pyridazin-2-yl]-1H-pyrazol-4-yl}-amide (9 mg). (LC/MS (basic method): Rₜ 1.86, [M+H]⁺ 429)

Biological Activity

### Example 9

### Measurement of Activated CDK2/CyclinA Kinase Inhibitory Activity Assay (IC₅₀)

Compounds of the invention were tested for kinase inhibitory activity using the following protocol.

Activated CDK2/CyclinA (Brown et al, Nat. Cell Biol., 1, pp 438-443, 1999; Lowe, E. D., et al Biochemistry, 41, pp 15625-15634, 2002) is diluted to 125 pM in 2.5× strength assay buffer (50 mM MOPS pH 7.2, 62.5 mM β-glycerophosphate, 12.5 mM EDTA, 37.5 mM MgCl₂, 112.5 mM ATP, 2.5 mM DTT, 2.5 mM sodium orthovanadate, 0.25 mg/ml bovine serum albumin), and 10 μl mixed with 10 μl of histone substrate mix (60 μl bovine histone H1 (Upstate Biotechnology, 5 mg/ml), 940 μl H₂O, 35 μCi γ³³P-ATP) and added to 96 well plates along with 5 μl of various dilutions of the test compound in DMSO (up to 2.5%). The reaction is allowed to proceed for 2 to 4 hours before being stopped with an excess of ortho-phosphoric acid (5 μl at 2%).

γ³³P-ATP which remains unincorporated into the histone H1 is separated from phosphorylated histone H1 on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds.

The % inhibition of the CDK2 activity is calculated and plotted in order to determine the concentration of test compound required to inhibit 50% of the CDK2 activity (IC₅₀).

### Example 10

### Measurement of Activated CDK1/CyclinB Kinase Inhibitory Activity Assay (IC₅₀)

CDK1/CyclinB assay is identical to the CDK2/CyclinA above except that CDK1/CyclinB (Upstate Discovery) is used and the enzyme is diluted to 6.25 nM.

The compounds of Examples 1, 4, 6 and 7 have IC₅₀ values of less than 1 μM against CDK1 and 2 activity.

### Example 11

### Aurora Kinase Inhibitory Activity Assay

AuroraA (Upstate Discovery) is diluted to 101 nM in 25 mM MOPS, pH 7.00, 25 mg/ml BSA, 0.0025% Brij-35, 1.25% glycerol, 0.5 mM EDTA, 25 mM MgCl₂, 0.025% β-mercaptoethanol, 37.5 mM ATP and 10 μl mixed with 10 μl of substrate mix. The substrate mix is 500 μM Kemptide peptide (LRRASLG, Upstate Discovery) in 1 ml of water with 35 μCi γ³³P-ATP. Enzyme and substrate are added to 96 well plates along with 5 μl of various dilutions of the test compound in DMSO (up to 2.5%). The reaction is allowed to proceed for 30 minutes before being stopped with an excess of ortho-phosphoric acid (5 μl at 2%). The filtration procedure is as for Activated CDK2/CyclinA assay above.

The compounds of Examples 1 to 6 and 8 have IC₅₀ values of less than 10 μM against Aurora A kinase.

### Example 12

### GSK3-B Kinase Inhibitory Activity Assay

GSK3-β (Upstate Discovery) are diluted to 7.5 nM in 25 mM MOPS, pH 7.00, 25 mg/ml BSA, 0.0025% Brij-35, 1.25% glycerol, 0.5 mM EDTA, 25 mM MgCl₂, 0.025% β-mercaptoethanol, 37.5 mM ATP and 10 μl mixed with 10 μl of substrate mix. The substrate mix for GSK3-β is 12.5 μM phospho-glycogen synthase peptide-2 (Upstate Discovery) in 1 ml of water with 35 μCi γ³P-ATP. Enzyme and substrate are added to 96 well plates along with 511 of various dilutions of the test compound in DMSO (up to 2.5%). The reaction is allowed to proceed for 3 hours (GSK3-β) before being stopped with an excess of ortho-phosphoric acid (5 μl at 2%). The filtration procedure is as for Activated CDK2/CyclinA assay above.

### Example 13

### Anti-Proliferative Activity

The anti-proliferative activities of compounds of the invention can be determined by measuring the ability of the compounds to inhibition of cell growth in a number of cell lines. Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds for a further 72 hours. At the end of the incubation period 10% (v/v) Alamar Blue is added and incubated for a further 6 hours prior to determination of fluorescent product at 535 nM ex/590 nM em. In the case of the non-proliferating cell assay cells are maintained at confluence for 96 hour prior to the addition of inhibitor compounds for a further 72 hours. The number of viable cells is determined by Alamar Blue assay as before. All cell lines can be obtained from ECACC (European Collection of cell Cultures).

By following the protocol set out above, compounds of the invention were found to inhibit cell growth in the HCT-116 cell line.

### Example 14

### Pharmaceutical Formulations

(i) Tablet Formulation

A tablet composition containing a compound of the formula (I) is prepared by mixing 50 mg of the compound with 197 mg of lactose (BP) as diluent, and 3 mg magnesium stearate as a lubricant and compressing to form a tablet in known manner.

(ii) Capsule Formulation

A capsule formulation is prepared by mixing 100 mg of a compound of the formula (I) with 100 mg lactose and filling the resulting mixture into standard opaque hard gelatin capsules.

### Example 15

### Determination of Antifungal Activity

The antifungal activity of the compounds of the formula (I) is determined using the following protocol.

The compounds are tested against a panel of fungi including Candida parpsilosis, Candida tropicalis, Candida albicans-ATCC 36082 and Cryptococcus neoformans. The test organisms are maintained on Sabourahd Dextrose Agar slants at 4° C. Singlet suspensions of each organism are prepared by growing the yeast overnight at 27° C. on a rotating drum in yeast-nitrogen base broth (YNB) with amino acids (Difco, Detroit, Mich.), pH 7.0 with 0.05 M morpholine propanesulphonic acid (MOPS). The suspension is then centrifuged and washed twice with 0.85% NaCl before sonicating the washed cell suspension for 4 seconds (Branson Sonifier, model 350, Danbury, Conn.). The singlet blastospores are counted in a haemocytometer and adjusted to the desired concentration in 0.85% NaCl.

The activity of the test compounds is determined using a modification of a broth microdilution technique. Test compounds are diluted in DMSO to a 1.0 mg/ml ratio then diluted to 64 μg/ml in YNB broth, pH 7.0 with MOPS (Fluconazole is used as the control) to provide a working solution of each compound. Using a 96-well plate, wells 1 and 3 through 12 are prepared with YNB broth, ten fold dilutions of the compound solution are made in wells 2 to 11 (concentration ranges are 64 to 0.125 μg/ml). Well 1 serves as a sterility control and blank for the spectrophotometric assays. Well 12 serves as a growth control. The microtitre plates are inoculated with 10 μl in each of well 2 to 11 (final inoculum size is 104 organisms/ml). Inoculated plates are incubated for 48 hours at 35° C. The IC50 values are determined spectrophotometrically by measuring the absorbance at 420 nm (Automatic Microplate Reader, DuPont Instruments, Wilmington, Del.) after agitation of the plates for 2 minutes with a vortex-mixer (Vorte-Genie 2 Mixer, Scientific Industries, Inc., Bolemia, N.Y.). The IC50 endpoint is defined as the lowest drug concentration exhibiting approximately 50% (or more) reduction of the growth compared with the control well. With the turbidity assay this is defined as the lowest drug concentration at which turbidity in the well is <50% of the control (IC50). Minimal Cytolytic Concentrations (MCC) are determined by sub-culturing all wells from the 96-well plate onto a Sabourahd Dextrose Agar (SDA) plate, incubating for 1 to 2 days at 35° C. and then checking viability.

### Example 17

### Protocol for the Biological Evaluation of Control of In Vivo Whole Plant Fungal Infection

Compounds of the formula (I) are dissolved in acetone, with subsequent serial dilutions in acetone to obtain a range of desired concentrations. Final treatment volumes are obtained by adding 9 volumes of 0.05% aqueous Tween-20 ™ or 0.01% Triton X-100™, depending upon the pathogen.

The compositions are then used to test the activity of the compounds of the invention against tomato blight (Phytophthora infestans) using the following protocol. Tomatoes (cultivar Rutgers) are grown from seed in a soil-less peat-based potting mixture until the seedlings are 10-20 cm tall. The plants are then sprayed to run-off with the test compound at a rate of 100 ppm. After 24 hours the test plants are inoculated by spraying with an aqueous sporangia suspension of Phytophthora infestans, and kept in a dew chamber overnight. The plants are then transferred to the greenhouse until disease develops on the untreated control plants.

Similar protocols are also used to test the activity of the compounds of the invention in combating Brown Rust of Wheat (Puccinia), Powdery Mildew of Wheat (Ervsiphe vraminis), Wheat (cultivar Monon), Leaf Blotch of Wheat (Septoria tritici), and Glume Blotch of Wheat (Leptosphaeria nodorum).

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-125. (canceled)

126. A compound of the formula (I): 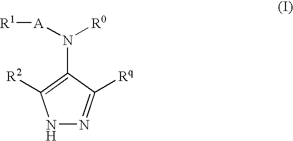
or salts or solvates or N-oxides thereof;
wherein:
R^{q} is selected from groups (a), (b) and (c): 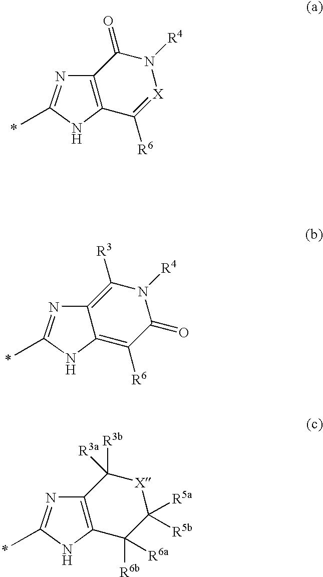
the asterisk denoting the point of attachment to the pyrazole ring;
X is N or CR⁵;
X″ is NR⁴, O, S or S(O);
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³, R⁵ and R⁶ are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; and a group R^{a}-R^{b};
R^{3a}, R^{3b}, R^{5a}, and R^{5b} are the same or different and each is selected from hydrogen, trifluoromethyl, cyano, carboxy, carbocyclic and heterocyclic groups having from 3 to 12 ring members; and a group R^{a}-R^{b};
R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹
R^{4′} is selected from hydrogen, trifluoromethyl, carbocyclic and heterocyclic groups having from 3 to 12 ring members; and a group R^{d′}-R^{e′} wherein R^{d′} is a bond, CO, C(X²)X¹, SO, SO₂, or SO₂NR^{c}; and R^{e′} is selected from carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{6a} and R^{6b} are the same or different and each is selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; and a group R^{a}-R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

127. A compound according to claim 126 where R⁰ is hydrogen; A is —(CH₂)ₘ—(B)ₙ—, wherein B is C═O or NR^{g}(C═O), R^{g} is hydrogen, m is 0, 1 or 2; and n is 0 or 1.

128. A compound according to claim 127 wherein B is C═O and m is 0, and n is 1.

129. A compound according to claim 126 wherein R¹ is a monocyclic or bicyclic carbocyclic or heterocyclic group having from 3 to 12 ring members and wherein the carbocyclic or heterocyclic group R¹ is unsubstituted or bears 1, 2, 3 or 4 substituents selected from the group R¹⁰, where R¹⁰ is selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹; where R^{c} is selected from hydrogen and C₁₋₄ hydrocarbyl; and X¹ is O, S or NR^{c} and X² is ═O, ═S or ═NR^{c}.

130. A compound according to claim 126 wherein R² is hydrogen, chlorine or methyl.

131. A compound according to claim 130 wherein R² is hydrogen.

132. A compound according to claim 126 having the formula (Ia) or (Ib): 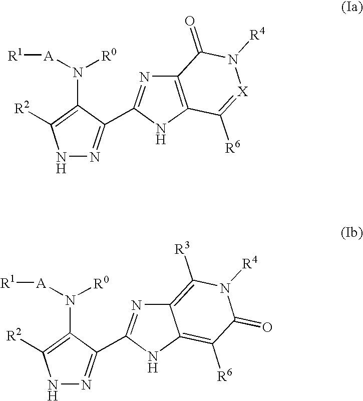
or salts or solvates or N-oxides thereof;
wherein
X is N or CR⁵;
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³, R⁵ and R⁶ are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c};
R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

133. A compound according to claim 132 wherein R³, R⁵ and R⁶ are all hydrogen.

134. A compound according to claim 132 wherein R⁴ is selected from hydrogen or a group R^{d}-R^{e} wherein R^{d} is a bond and R^{e} is a C₁₋₄ hydrocarbyl group optionally substituted by one or more substituents selected from hydroxy, alkoxy, halogen preferably fluorine, carbocyclic and heterocyclic groups having from 3 to 7 ring members.

135. A compound according claim 134 wherein R⁴ is hydrogen, or methyl, ethyl or propyl optionally substituted with an unsubstituted 6-membered non-aromatic heterocycle or an unsubstituted 6-membered carbocycle.

136. A compound according to claim 132 wherein X is CR⁵.

137. A compound according to claim 132 wherein X is N.

138. A compound according to claim 132 wherein R¹ is (i) a substituted or unsubstituted phenyl ring; or (ii) a non-aromatic group selected from monocyclic cycloalkyl groups, monocyclic oxacycloalkyl groups and monocyclic azacycloalkyl groups such as piperidinyl.

139. A compound according to claim 132 wherein R¹ is selected from 2,6-difluorophenyl, 2-methoxy-5-chloro-phenyl, tetrahydropyran, 4-(2-methyl-5-furan-3-ylmethyl)-morpholine, and 4-methyltetrahydropyran.

140. A compound according to claim 132 having formula (II) or formula (III): 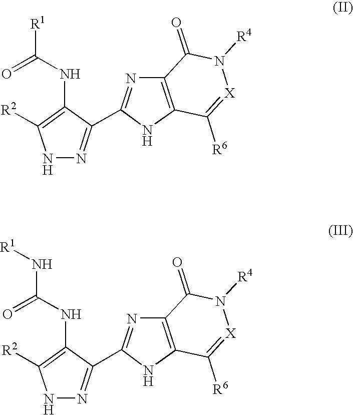
wherein R¹, R², and R⁴ to R⁶ are as defined in claim 132.

141. A compound according to claim 126 having the formula (Ic): 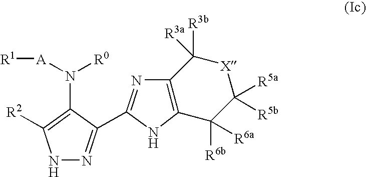
or salts or solvates or N-oxides thereof;
wherein
X″ is NR^{4′}, O, S or S(O);
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R^{3a}, R^{3b}, R^{5a}, and R^{5b} are the same or different and each is selected from hydrogen, trifluoromethyl, cyano, carboxy, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X¹), C(X¹)X¹ or X¹C(X²)X¹;
R^{c} is selected from hydrogen and C₁₋₄ hydrocarbyl; and
X¹ is O, S or NR^{c} and X² is ═O, ═S or ═NR^{c};
R^{4′} is selected from hydrogen, trifluoromethyl, carbocyclic and heterocyclic groups having from 3 to 12 ring members; a group R^{d′}-R^{e′} wherein R^{d′} is a bond, CO, C(X²)X¹, SO, SO₂, or SO₂NR^{c}; and R^{e′} is selected from carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{6a} and R^{6b} are the same or different and each is selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; a group R^{a}-R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹.

142. A compound according to claim 141 wherein R^{3a}, R^{3b}, R^{5a}, and R^{5b} each are hydrogen.

143. A compound according claim 141 wherein R^{4′} is selected from hydrogen or a group R^{d′}-R^{e′} wherein R^{d′} is a bond, CO, C(X²)X¹, or SO₂; and R^{e′} is selected from carbocyclic and heterocyclic groups having from 3 to 7 ring members, and a C₁₋₄ hydrocarbyl group optionally substituted by one or more substituents selected from hydroxy, alkoxy, halogen, amino, mono- or di-C₁₋₄ hydrocarbylamino, carbocyclic and heterocyclic groups having from 3 to 7 ring members.

144. A compound according to claim 143 wherein R^{4′} is selected from hydrogen or a group R^{d′}-R^{e′} wherein R^{d′} is a bond and R^{e′} is a C₁₋₄ hydrocarbyl group optionally substituted by one or more substituents selected from hydroxy, alkoxy, halogen preferably fluorine, carbocyclic and heterocyclic groups having from 3 to 7 ring members.

145. A compound according to claim 144 wherein R^{4′} is selected from hydrogen, unsubstituted methyl, unsubstituted 2-methoxy-ethyl, unsubstituted 2-fluoro-ethyl, and unsubstituted 2,2-difluoro-ethyl.

146. A compound according to claim 141 wherein R^{6a} and R^{6b} are both hydrogen.

147. A compound according to claim 141 wherein X″ is O or NR^{4′}.

148. A compound according to claim 141 wherein R¹ is selected from 2,6-difluorophenyl, 2-fluoro-6-methoxyphenyl, 2,6-dichlorophenyl, 2,4,6-trifluorophenyl, 2,3-dihydro-benzo[1,4]furan-7-yl, cinnolin-4-yl, benzo[c]isoxazole-3-yl and pyrazolo[1,5-a]pyridin-3-yl.

149. A compound according to claim 141 wherein R¹ is a non-aromatic group selected from monocyclic cycloalkyl groups and azacycloalkyl groups.

150. A compound according to claim 141 having the formula (IV): 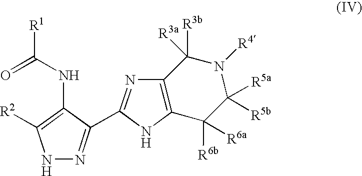
wherein R¹, R², R^{3a}, R^{3b}, R^{4′}, R^{5a}, R^{5b}, R^{6a} and R^{6b} are as defined in claim 141.

151. A compound according to claim 141 wherein X″ is S and R^{3a}, R^{3b}, R^{5a}, R^{5b}, R^{6a} and R^{6b} are each hydrogen.

152. A method for the prophylaxis or treatment of a disease state or condition mediated by a cyclin dependent kinase or an aurora kinase or glycogen synthase kinase-3, which method comprises administering to a subject in need thereof an effective amount of a compound of the formula (I): 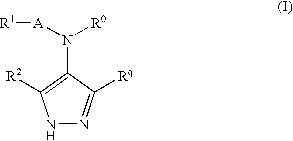
or salts or solvates or N-oxides thereof;
wherein:
R^{q} is selected from groups (a), (b) and (c): 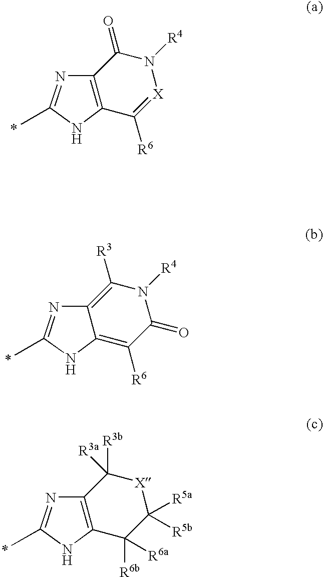
the asterisk denoting the point of attachment to the pyrazole ring;
X is N or CR⁵;
X″ is NR^{4′}, O, S or S(O);
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³, R⁵ and R⁶ are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; and a group R^{a}-R^{b};
R^{3a}, R^{3b}, R^{5a}, and R^{5b} are the same or different and each is selected from hydrogen, trifluoromethyl, cyano, carboxy, carbocyclic and heterocyclic groups having from 3 to 12 ring members; and a group R^{a}-R^{b};
R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹
R^{4′} is selected from hydrogen, trifluoromethyl, carbocyclic and heterocyclic groups having from 3 to 12 ring members; and a group R^{d′}-R^{e′} wherein R^{d′} is a bond, CO, C(X²)X¹, SO, SO₂, or SO₂NR^{c}; and R^{e′} is selected from carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{6a} and R^{6b} are the same or different and each is selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; and a group R^{a}-R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

153. A method for treating a disease or condition comprising or arising from abnormal cell growth in a mammal, which method comprises administering to the mammal in an amount effective in inhibiting abnormal cell growth a compound of formula (I) 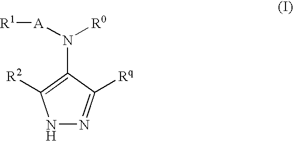
or salts or solvates or N-oxides thereof;
wherein:
R^{q} is selected from groups (a), (b) and (c): 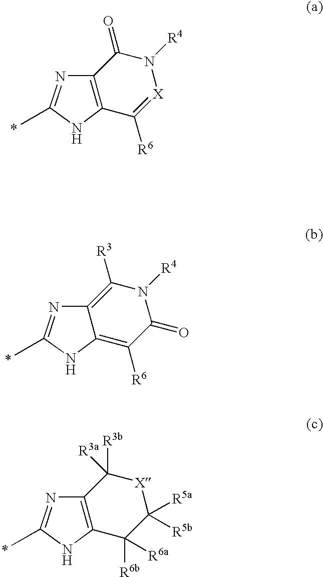
the asterisk denoting the point of attachment to the pyrazole ring;
X is N or CR⁵;
X″ is NR^{4′}, O, S or S(O);
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³, R⁵ and R⁶ are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; and a group R^{a}-R^{b};
R^{3a}, R^{3b}, R^{5a}, and R^{5b} are the same or different and each is selected from hydrogen, trifluoromethyl, cyano, carboxy, carbocyclic and heterocyclic groups having from 3 to 12 ring members; and a group R^{a}-R^{b};
R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹
R^{4′} is selected from hydrogen, trifluoromethyl, carbocyclic and heterocyclic groups having from 3 to 12 ring members; and a group R^{d′}-R^{e′} wherein R^{d′} is a bond, CO, C(X²)X¹, SO, SO₂, or SO₂NR^{c}; and R^{e′} is selected from carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{6a} and R^{6b} are the same or different and each is selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; and a group R^{a}-R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

154. A method according to claim 153 wherein the disease state or condition is selected from proliferative disorders, viral infections, autoimmune diseases and neurodegenerative diseases.

155. A method according to claim 154 wherein the disease state is a cancer selected from breast cancer, ovarian cancer, colon cancer, prostate cancer, oesophageal cancer, squamous cancer, and non-small cell lung carcinomas.

156. A pharmaceutical composition comprising a compound of the formula (I): 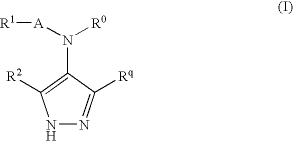
or salts or solvates or N-oxides thereof;
wherein:
R^{q} is selected from groups (a), (b) and (c): 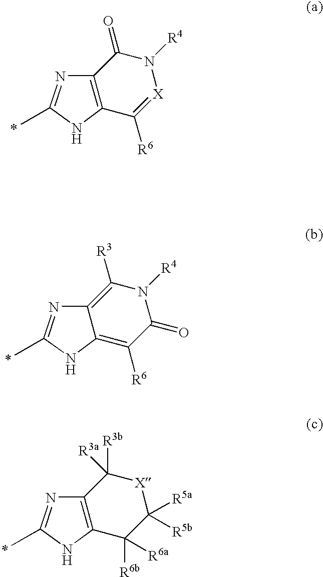
the asterisk denoting the point of attachment to the pyrazole ring;
X is N or CR⁵;
X″ is NR^{4″}, O, S or S(O);
A is a bond or —(CH₂)ₘ—(B)ₙ—;
B is C═O, NR^{g}(C═O) or O(C═O) wherein R^{g} is hydrogen or C₁₋₄ hydrocarbyl optionally substituted by hydroxy or C₁₋₄ alkoxy;
m is 0, 1 or 2;
n is 0 or 1;
R⁰ is hydrogen or, together with NR^{g} when present, forms a group —(CH₂)ₚ— wherein p is 2 to 4;
R¹ is hydrogen, a carbocyclic or heterocyclic group having from 3 to 12 ring members, or an optionally substituted C₁₋₈ hydrocarbyl group;
R² is hydrogen, halogen, methoxy, or a C₁₋₄ hydrocarbyl group optionally substituted by halogen, hydroxyl or methoxy;
R³, R⁵ and R⁶ are the same or different and each is selected from hydrogen, halogen, hydroxy, trifluoromethyl, cyano, nitro, carboxy, amino, mono- or di-C₁₋₄ hydrocarbylamino, carbocyclic and heterocyclic groups having from 3 to 12 ring members; and a group R^{a}-R^{b};
R^{3a}, R^{3b}, R^{5a}, and R^{5b} are the same or different and each is selected from hydrogen, trifluoromethyl, cyano, carboxy, carbocyclic and heterocyclic groups having from 3 to 12 ring members; and a group R^{a}-R^{b};
R⁴ is selected from hydrogen, trifluoromethyl, carboxy, carbocyclic and heterocyclic groups having from 3 to 12 ring members; a group R^{d}-R^{e} wherein R^{d} is a bond, CO, C(X²)X¹, S, SO, SO₂, or SO₂NR^{c}; and R^{e} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹
R^{4′} is selected from hydrogen, trifluoromethyl, carbocyclic and heterocyclic groups having from 3 to 12 ring members; and a group R^{d′}-R^{e′} wherein R^{d′} is a bond, CO, C(X²)X¹, SO, SO₂, or SO₂NR^{c}; and R^{e′} is selected from carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{6a} and R^{6b} are the same or different and each is selected from hydrogen, halogen, hydroxy, trifluoromethyl, cyano, carboxy, amino, mono- or di-C₁₋₄ hydrocarbylamino, carbocyclic and heterocyclic groups having from 3 to 12 ring members; and a group R^{a}-R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.
